(19) United States
(12) Patent Application Publication
(10) Pub. No.: US 2025/0144116 A1
(43) Pub. Date: May 8, 2025

(54) CERTAIN N-(1-CYAN0-2-PHENYLETHYL]-1,4-OXAZEPANE-2-CARBOXAMIDES FOR TREATING HIDRADENITIS SUPPURATIVA

(71) Applicant: Insmed Incorporated, Bridgewater, NJ (US)

(72) Inventors: Ariel TEPER, Bridgewater, NJ (US); Andrea MAES, Bridgewater, NJ (US); Marcela VERGARA, Bridgewater, NJ (US); Christina RAMIREZ, Bridgewater, NJ (US)

(73) Assignee: Insmed Incorporated, Bridgewater, NJ (US)

(21) Appl. No.: 18/838,823

(22) PCT Filed: Feb. 16, 2023
(86) PCT No.: PCT/US2023/062731

§ 371 Date: Aug. 15, 2024

Related U.S. Application Data
(60) Provisional application No. 63/310,751, filed on Feb. 16, 2022.

(51) Int. Cl. A61K 31/553; A61K 45/06

(52) U.S. Cl. CPC A61K 31/553; A61K 45/06

(57) ABSTRACT

The present disclosure relates to methods for treating hidradenritis suppurativa with a composition comprising an effective amount of a N-(1-cyano-2-phenylethyl)-1, 4-oxazepane-2-carboxamide DPP1 inhibitor compound of Formula (1) or a pharmaceutically acceptable salt thereof. In one embodiment, the compound of Formula (I) is (2S)—N-{(1S)-1-cyano-2-[4-(3-methyl-2-oxo-2,3-dihydro-1, 3-benzoxazol-5-yl)phenyl]ethyl}-1,4-oxazepane-2-carboxamide (brensocatib).

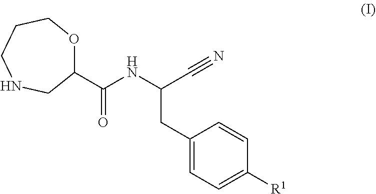

### CROSS REFERENCE TO RELATED APPLICATION

This application claims priority to U.S. Provisional Application Ser. No. 63/310,751, filed Feb. 16, 2022, the disclosure of which is incorporated by reference herein in its entirety.

### BACKGROUND

Hidradenitis suppurativa (HS) is a chronic relapsing inflammatory disorder. The symptoms include skin lesions that are often associated hair follicles, and may be painful, inflamed and/or swollen. In some cases, when the skin lesions heal, they can recur, and may lead to tunnels under the skin and progressive scarring. Since HS is a chronic condition, it can persist for many years and also, worsen over time, with serious effects on quality of life, physochological and emotional well-being. In fact, HS pateints have increased rates of anxiety and depression with a risk of suicide two and a half times that of the general population.

Patients with HS are categorized according to disease severity, termed Hurley staging, as mild (Stage I), moderate (Stage II), or severe (Stage III). Although more than 200,000 cases of HS are diagnosed in the U.S. per year, this disease can be difficult to diagnose and requires specialized care. HS may be mistaken for an infection, an ingrown hair or other conditions. Moreover, current treatment options are limited and lack efficacy.

Thus, there is an unmet need to develop more effective therapies for HS patients, particularly treatments that target the underlying inflammation that results in the symptomatic skin lesions.

### SUMMARY

The present invention, in one aspect, provides a method of treating hidradenitis suppurativa (HS) in a subject in need thereof, comprising: administering to the subject for an administration period, a pharmaceutical composition comprising an effective amount of a compound of Formula (I) or a pharmaceutically acceptable salt thereof,

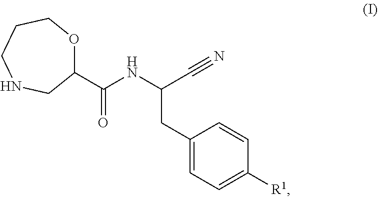

wherein
R¹ is

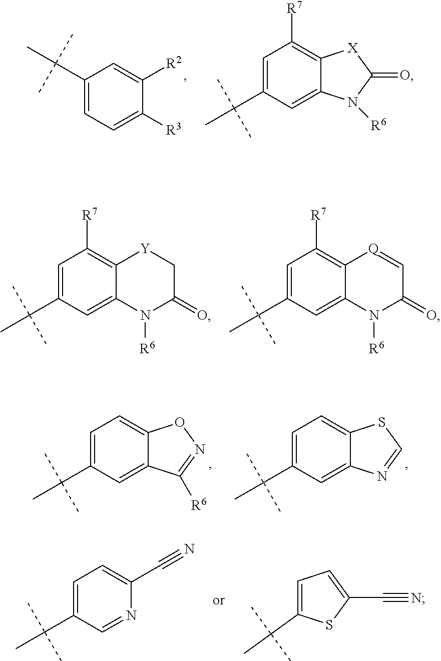

R² is hydrogen, F, Cl, Br, OSO₂C₁₋₃alkyl, or C₁₋₃alkyl;
R³ is hydrogen, F, Cl, Br, CN, CF₃, SO₂C₁₋₃alkyl, CONH₂ or SO₂NR⁴R⁵,
wherein R⁴ and R⁵ together with the nitrogen atom to which they are attached form an azetidine, pyrrolidine or piperidine ring;
X is O, S or CF₂;
Y is O or S;
Q is CH or N;
R⁶ is C₁₋₃alkyl, wherein said C₁₋₃alkyl is optionally substituted by 1, 2 or 3 F and optionally by one substituent selected from the group consisting of OH, OC₁₋₃alkyl, N(C₁₋₃alkyl)₂, cyclopropyl, and tetrahydropyran; and
R⁷ is hydrogen, F, Cl or CH₃.

In one embodiment of the methods provided herein, the compound of Formula (I) is an S,S diastereomer, i.e., the compound of Formula (I) has the following stereochemistry:

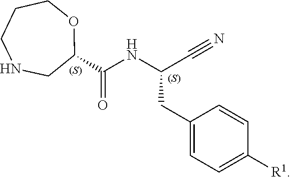

The other diastereomeric forms of the compounds of Formula (I) are also contemplated by the present invention. For example, in one embodiment, the compound of Formula (I) is the R,R diastereomer. In another embodiment, the compound of Formula (I) is the R,S diastereomer. In even another embodiment, the compound of Formula (I) is the S,R diastereomer.

In some embodiments, R¹ is

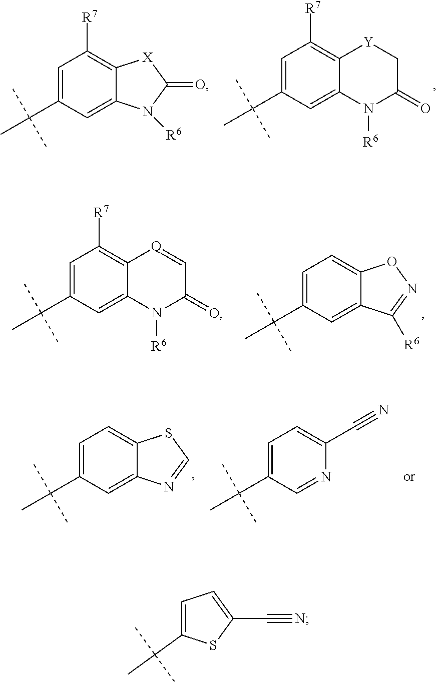

X is O, S or CF₂;
Y is O or S;
Q is CH or N;
R⁶ is C₁₋₃alkyl, wherein said C₁₋₃alkyl is optionally substituted by 1, 2 or 3 F and optionally by one substituent selected from the group consisting of OH, OC₁₋₃alkyl, N(C₁₋₃alkyl)₂, cyclopropyl, and tetrahydropyran; and
R⁷ is hydrogen, F, Cl or CH₃.

In some embodiments, R¹ is

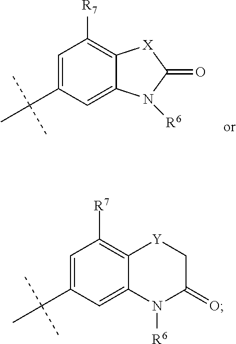

X is O, S or CF₂;
Y is O or S;
R⁶ is C₁₋₃alkyl, wherein said C₁₋₃alkyl is optionally substituted by 1, 2 or 3 F and optionally by one substituent selected from the group consisting of OH, OC₁₋₃alkyl, N(C₁₋₃alkyl)₂, cyclopropyl, and tetrahydropyran; and
R⁷ is hydrogen, F, Cl or CH₃.

In some embodiments, R¹ is

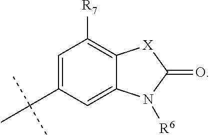

In some embodiments, X is O; R⁶ is C₁₋₃alkyl; and R⁷ is hydrogen.

In some embodiments, R¹ is

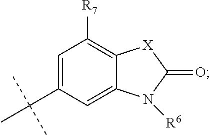

X is O; R⁶ is C₁₋₃alkyl, wherein said C₁₋₃ alkyl is optionally substituted by 1, 2 or 3 F; and R⁷ is hydrogen.

In some embodiments, R¹ is

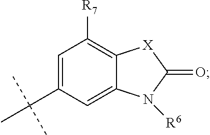

X is O; R⁶ is C₁₋₃alkyl; and R⁷ is hydrogen.

In some embodiments, the compound of Formula (I) is (2S)—N-{(1S)-1-cyano-2-[4-(3-methyl-2-oxo-2,3-dihydro-1,3-benzoxazol-5-y-l)phenyl]ethyl}-1,4-oxazepane-2-carboxamide, referred to herein by its international nonproprietary name (INN), brensocatib (and formerly known as INS1007 and AZD7986):

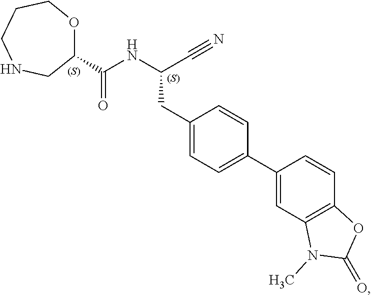

or a pharmaceutically acceptable salt thereof.

In one embodiment, the HS is Hurley Stage I HS, Hurley Stage II HS or Hurley Stage III HS.

In one embodiment, a method of treating HS disclosed herein comprises diminishing the severity of, or eliminating one or more symptoms of HS of the subject, during or subsequent to the administration period, compared to the one or more symptoms prior to the administration period. The one or more symptoms, in one embodiment, are: (a) one or more skin lesions; (b) one or more skin abscesses; (c) one or more fistulas; (d) one or more sinus tracts; (e) skin scarring; (f) anxiety; (g) depression; (h) suicidal thoughts; or (i) any combinations thereof. In a further embodiment, the one or more fistulas comprise one or more draining fistulas.

In one embodiment, the subject has not been treated with an anti-TNFα agent (e.g., an anti-TNFα antibody) previously.

In one embodiment, the subject is not responsive to a prior, different HS treatment, e.g., administration of a systemic antibiotic.

In one embodiment, the subject achieves a Hidrahenitis Suppurative Clinical Response 50 (HiSCR50) during or subsequent to the administration period, wherein the HiSCR50 is at least a 50% reduction in total AN count, with no increase in skin abscess count, and no increase in draining fistula count, as compared to a total AN count, a skin abscess count, and a draining fistula count of the subject, respectively, prior to the administration period.

In one embodiment, the subject achieves a Hidrahenitis Suppurative Clinical Response 75 (HiSCR75) during or subsequent to the administration period, wherein the HiSCR75 is at least a 75% reduction in total AN count, with no increase in skin abscess count, and no increase in draining fistula count, as compared to a total AN count, a skin abscess count, and a draining fistula count of the subject, respectively, prior to the administration period.

In one embodiment, a method of treating HS disclosed herein comprises reducing a Hidradenitis Suppurativa—Investigator's Global Assessment (HS-IGA) score of the subject during or subsequent to the administration period, as compared to an HS-IGA score of the subject prior to the administration period. In a further embodiment, the HS-IGA score of the subject is reduced ≥2 points during or subsequent to the administration period.

In one embodiment, a method of treating HS disclosed herein comprises reducing a skin abscess and inflammatory skin nodule count (AN count) of the subject during or subsequent to the administration period, as compared to an AN count of the subject prior to the administration period.

In one embodiment, a method of treating HS disclosed herein comprises reducing or maintaining a fistula count of the subject during or subsequent to the administration period, as compared to a fistula count of the subject prior to the administration period. In a further embodiment, the fistula count is a draining fistula count.

In one embodiment, a method of treating HS disclosed herein comprises reducing an occurrence of HS flares in the subject during or subsequent to the administration period, as compared to an occurrence of HS flares in the subject prior to the administration period.

In one embodiment, a method of treating HS disclosed herein comprises reducing an International hidradenitis suppurativa severity (IHS4) score of the subject during or subsequent to the administration period, as compared to an IHS4 score of the subject prior to the administration period. In a further embodiment, the IHS4 score of the subject is ≥4 prior to the administration period, e.g., ≥11 prior to the administration period.

In one embodiment, a method of treating HS disclosed herein comprises reducing a Dermatology Life Quality Index (DLQI) score of the subject during or subsequent to the administration period, as compared to a DLQI score of the subject prior to the administration period.

In one embodiment, a method of treating HS disclosed herein comprises reducing a Patient's Global Assessment of Skin Pain (PGA-SP) score of the subject during or subsequent to the administration period, as compared to a PGA-SP score of the subject prior to the administration period.

In one embodiment, a method of treating HS disclosed herein comprises reducing a Hidradenitis Suppurativa Quality of Life (HiSQOL) score of the subject during or subsequent to the administration period, as compared to a HiSQOL score of the subject prior to the administration period.

In one embodiment, the pharmaceutical composition is administered once-a-day during the administration period.

In one embodiment, the pharmaceutical composition is administered orally during the administration period.

In one embodiment, the compound of Formula (I) is present in the pharmaceutical composition from about 1 mg to about 100 mg, e.g., about 10 mg, about 25 mg, or about 40 mg. In a further embodiment, the compound of Formula (I) is brensocatib.

In one embodiment, the administration period is the entire life of the subject following the initial diagnosis of HS. In another embodiment, the administration period is from about 1 year to about 50 years, from about 1 year to about 30 years, or from about 1 year to about 10 years.

In one embodiment, a method of treating HS disclosed herein comprises administering a secondary therapy to the subject. The secondary therapy may be administered: (a) prior to the administration period, (b) during or subsequent to the administration period, or (c) concurrently with administering the pharmaceutical composition. In some embodiments, the the secondary therapy comprises one or more of the following: an antibiotic, a steroid, a biologic, a hormone, a retinoid, an anti-inflammatory drug, a surgical intervention, or any combination thereof.

### BRIEF DESCRIPTION OF THE FIGURE

FIG. 1 is a schematic diagram of the study design set forth in Example 1. IHS4=International Hidradenitis Suppurativa Severity Score 4; PD=pharmacodynamic; Q2W=every 2 weeks; Q4W=every 4 weeks; QD=once daily; TNF=tumor necrosis factor.

### DETAILED DESCRIPTION

Hidradenitis suppurativa (HS) is a chronic inflammatory skin disease primarily affecting apocrine gland-rich areas of the body and presenting with skin nodules, skin abscesses, sinus tracts, and skin scarring. Neutrophils are the predominant leukocyte infiltrate in HS-related skin lesions. The lysosomal cysteine proteinase dipeptidyl peptidase 1 (DPP1, also known as cathepsin C) activates neutrophil serine proteases (NSPs) which, when released from neutrophils, are pro-inflammatory and generally destructive to the surrounding tissues. Without being bound by theory, it is thought that inhibition of DPP1 activity (e.g., by administration of the compounds disclosed herein) in patients with HS can lead to a reduction in the formation of active NSPs, thereby preventing the propagation of the inflammatory response.

Aspects of the invention provide reversible inhibitors of dipeptidyl peptidase 1 (DPP1) or cathepsin C, and methods of use thereof for treating HS. DPP1 catalyses excision of dipeptides from the N-terminus of protein and peptide substrates. Through this enzymatic function, DPP1 activates many serine proteases in immune/inflammatory cells, such as, neutrophil serine proteases (NSPs), including neutrophil elastase (NE), proteinase 3 (PR3), cathepsin G (CatG), and neutrophil serine protease 4 (NSP4). Neutrophil serine proteases can mediate neutrophilic inflammation, which is often seen in patients with hidradenitis suppurativa. Thus, in some embodiments, the methods of use are methods of treating hidradenitis suppurativa.

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

Following long-standing patent law convention, the terms “a”, “an”, and “the” refer to “one or more” when used in this application, including the claims. Thus, for example, reference to “a carrier” includes mixtures of one or more carriers, two or more carriers, and the like and reference to “the method” includes reference to equivalent steps and/or methods known to those skilled in the art, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the present application. Generally, the term “about”, as used herein in references to a measurable value such as an amount of weight, time, dose, etc. is meant to encompass values within an acceptable degree of variability in the art. In some embodiments, degree of variability is based on FDA guidelines.

Also as used herein, “and/or” refers to and encompasses any and all possible combinations of one or more of the associated listed items, as well as the lack of combinations when interpreted in the alternative (“or”).

As used herein, “C₁₋₃” means a carbon group having 1, 2 or 3 carbon atoms.

The term “alkyl”, unless otherwise noted, includes both straight and branched chain alkyl groups and may be substituted or non-substituted. “Alkyl” groups include, but are not limited to, methyl, ethyl, n-propyl, i-propyl, butyl, pentyl.

The term “pharmaceutically acceptable”, unless otherwise noted, is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

As used herein, the terms “treatment” or “treating,” and “ameliorating” are used interchangeably. These terms refer to an approach for obtaining beneficial or desired results including but not limited to a therapeutic benefit and/or a prophylactic benefit. Therapeutic benefit refers to any therapeutically relevant improvement in or effect on one or more diseases, conditions, or symptoms under treatment. The term “treating” in one embodiment, includes: (1) preventing or delaying the appearance of clinical symptoms of the state, disorder or condition developing in the patient that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); (3) relieving the condition (for example, by causing regression, or reducing the severity of the state, disorder or condition or at least one of its clinical or subclinical symptoms).

The term “effective amount”, as used herein, refers to the amount of an agent (e.g., a compound(s) of Formula (I)) that is sufficient to achieve an outcome, for example, to effect beneficial or desired results. The effective amount may vary depending upon one or more of: the subject and disease severity being treated, the weight and age of the subject, the manner of administration and the like. The effective amount may refer to the amount of the agent administered to a subject in a single dosing session or multiple dosing sessions.

The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human. A subject's tissues, cells, or derivatives thereof, obtained in vivo or cultured in vitro are also encompassed. A human subject may be an adult, a teenager, a child (2 years to 14 years of age), an infant (1 month to 24 months), or a neonate (up to 1 month). In some embodiments, an adult subject is about 60 years or older, or about 65 years or older. In some embodiments, the subject is a pregnant woman or a woman intending to become pregnant.

As used herein, a “skin lesion” is a part of the skin that has an abnormal growth, appearance, or a combination thereof, as compared to the skin around it.

As used herein, a “skin abscess” is a pus-filled area associated with the skin tissue. In some embodiments, the skin abscess appears as a swelling on or below the surface of the skin. In some embodiments, the skin abscess has a diameter of at least about 10 mm. In some embodiments, the pus contains bacteria, white blood cells, and/or dead skin.

As used herein, a “fistula” is an abnormal channel between two cavities or surfaces. A fistula may be a draining fisula, or a non-draining fistula. In some embodiments, the fistula (e.g., the draining fistula) may ooze a fluid. Further details on fistulas can be found in Kimball et al. Journal of the European Academy of Dermatology and Venereology 2016, 30, 989-994, the contents of which are incorporated herein by reference in its entirety. In the present application, “fistula” is also referred to as “tunnel.” These two terms are used interchangeably.

As used herein, a “sinus tract” (or sinus) is a small, uncharacteristic channel in the body. In some embodiments, the sinus has one open draining end and the channel ends in a blind ending. For instance, in some embodiments, the sinus tract extends from a wound underneath the skin through soft tissue. In some embodiments, the sinus tract results in dead space, which can lead to skin abscess formation.

As used herein, a “skin nodule” is an elevated lesion on or in the skin. In some embodiments, skin nodules are solid and/or palpable masses. In some embodiments, a skin nodule is an inflammatory skin nodule. In some embodiments, the skin nodule is raised, three-dimensional, round, and/or infiltrated. In some embodiments, the diameter of the skin nodule is at least about 10 mm.

In one aspect, a method is provided for inhibiting dipeptidyl peptidase (DPP1) in a subject in need thereof, comprising: administering to the subject for an administration period, a pharmaceutical composition comprising an effective amount of a compound of Formula (I), or a pharmaceutically acceptable salt thereof. In some embodiments, the DPP1 is expressed by neutrophils. The subject has HS or is at risk of developing HS. In some embodiments, the administration results in the treatment or delay in the onset of HS in the subject.

In another aspect, a method of treating HS in a subject in need thereof is provided. The method comprises in one embodiment, administering to the subject for an administration period, a pharmaceutical composition comprising an effective amount of a compound of Formula (I), or a pharmaceutically acceptable salt thereof. In a further embodiment, the method comprises reducing neutrophilic inflammation in the subject.

The methods provided herein are not limited to the administration of the compound of the Formula (I). Other DPP1 inhibitors may be used in the methods disclosed herein, for example, DPP1 inhibitors described in Banerjee, et al., Bioorganic & Medicinal Chemistry Letters Volume 47, 1 Sep. 2021, 128202; Chinese Patent Application Publication No: CN112920124A; Chen et al. Journal of Medicinal Chemistry 64.16 (2021): 11857-11885; and International Patent Publication WO2022/020245, the contents of each of which are incorporated herein by reference in their entireties for all purposes.

In the methods of treating HS described herein, the HS is Hurley Stage I HS, Hurley Stage II HS or Hurley Stage III HS. In some embodiments, the HS is Hurley Stage I HS. In some embodiments, the HS is Hurley Stage II HS. In some embodiments, the HS is Hurley Stage III HS.

In one aspect, a method of treating hidradenitis suppurativa in a subject in need thereof is provided. The method comprises administering to the subject, for an administration period, a pharmaceutical composition comprising an effective amount of a compound of Formula (I), or a pharmaceutically acceptable salt thereof:

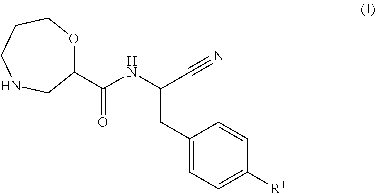

wherein
R¹ is

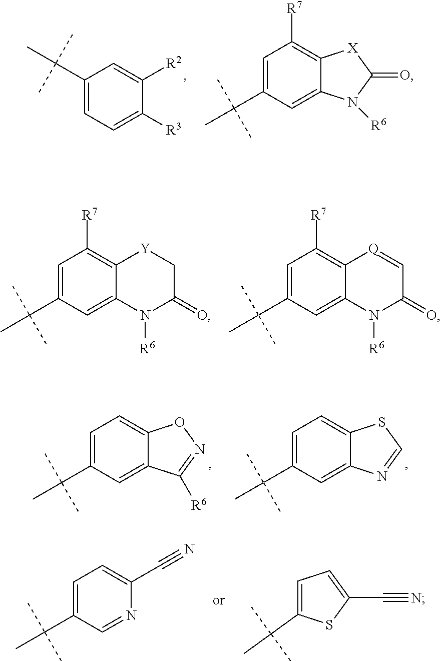

R² is hydrogen, F, Cl, Br, OSO₂C₁₋₃alkyl, or C₁₋₃alkyl;
R³ is hydrogen, F, Cl, Br, CN, CF₃, SO₂C₁₋₃alkyl, CONH₂ or SO₂NR⁴R⁵, wherein R⁴ and R⁵ together with the nitrogen atom to which they are attached form an azetidine, pyrrolidine or piperidine ring; or
R⁶ is C₁₋₃alkyl, optionally substituted by 1, 2 or 3 F and/or optionally by OH, OC₁₋₃alkyl, N(C₁₋₃alkyl)₂, cyclopropyl, or tetrahydropyran;
R⁷ is hydrogen, F, Cl or CH₃;
X is O, S or CF₂;
Y is O or S; and
Q is CH or N.

In one embodiment, the subject has not been previously treated with an anti-tumor necrosis factor alpha (TNFα) agent (e.g., an anti-TNFα antibody). In another embodiment of a method of treatment described herein, the HS is refractory HS, e.g., the subject is not responsive to a prior, different HS treatment. In a further embodiment, the prior different HS treatment comprises a systemic antibiotic.

In one embodiment of a method provided herein, the compound of Formula (I) is an S,S diastereomer. In other words, the compound of Formula (I) has the following stereochemistry:

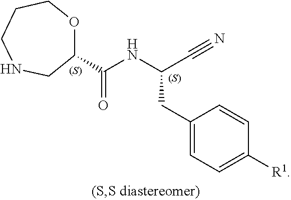

The other diastereomeric forms of compounds of Formula (I), and mixtures thereof, are also contemplated by the present invention. For example, in one embodiment, the compound of Formula (I) is the R,R diastereomer:

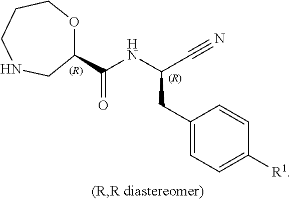

In another embodiment, the compound of Formula (I) is the R,S diastereomer:

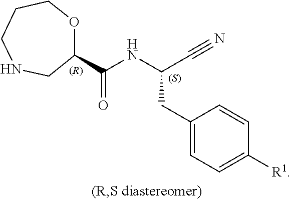

In even another embodiment, the compound of Formula (I) is the S,R diastereomer:

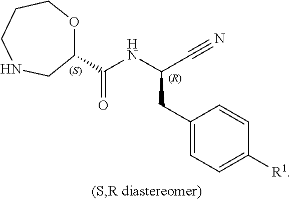

In one embodiment of a method provided herein, R¹ is

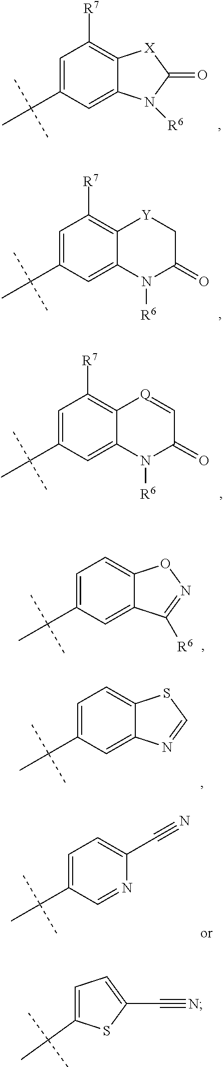

X is O, S or CF₂; Y is O or S; Q is CH or N; R⁶ is C₁₋₃alkyl, wherein said C₁₋₃alkyl is optionally substituted by 1, 2 or 3 F and optionally by one substituent selected from the group consisting of OH, OC₁₋₃alkyl, N(C₁₋₃alkyl)₂, cyclopropyl, and tetrahydropyran; and R⁷ is hydrogen, F, Cl or CH₃.

In one embodiment, R¹ is

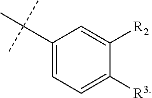

R² is hydrogen, F, Cl, Br, OSO₂C₁₋₃alkyl, or C₁₋₃ alkyl; R³ is hydrogen, F, Cl, Br, CN, CF₃, SO₂C₁₋₃alkyl, CONH₂ or SO₂NR⁴R⁵, wherein R⁴ and R⁵ together with the nitrogen atom to which they are attached form an azetidine, pyrrolidine or piperidine ring.

In a further embodiment, R¹ is

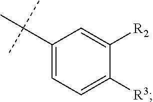

R² is hydrogen, F, Cl or C₁₋₃alkyl; and R³ is hydrogen, F, Cl, CN or SO₂C₁₋₃alkyl.

In still a further embodiment, R¹ is

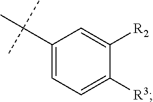

R² is hydrogen, F or C₁₋₃alkyl; and R³ is hydrogen, F or CN.

In another embodiment, R¹ is

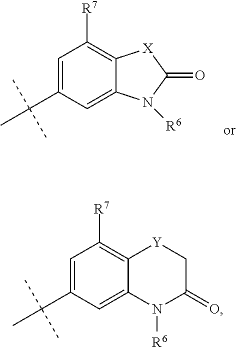

X is O, S or CF₂; Y is 0 or S; R⁶ is C₁₋₃alkyl, wherein the C₁₋₃alkyl is optionally substituted by 1, 2 or 3 F and optionally substituted by OH, OC₁₋₃alkyl, N(C₁₋₃alkyl)₂, cyclopropyl, or tetrahydropyran; and R⁷ is hydrogen, F, Cl or CH₃.

In one embodiment, R¹ is R⁶.

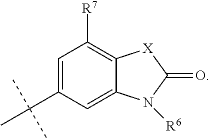

In another embodiment, R is

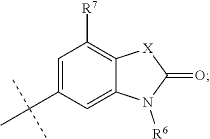

X is O; R⁶ is C₁₋₃alkyl; and R⁷ is hydrogen. In a further embodiment, R⁶ is methyl.

In one embodiment, R¹ is

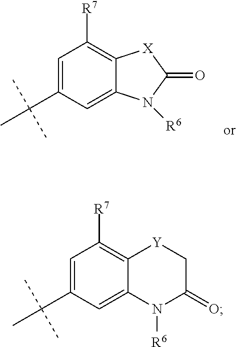

X is O, S or CF₂; Y is O or S; R⁶ is C₁₋₃alkyl, optionally substituted by 1, 2 or 3 F, and R⁷ is hydrogen, F, Cl or CH₃.

In one embodiment, R¹ is

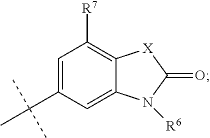

X is O, S or CF₂; R⁶ is C₁₋₃alkyl, wherein the C₁₋₃alkyl is optionally substituted by 1, 2 or 3 F; and R⁷ is hydrogen, F, Cl or CH₃.

In one embodiment, R¹ is

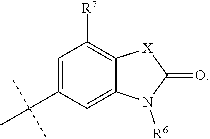

In one embodiment, X is O; R⁶ is C₁₋₃alkyl; and R⁷ is hydrogen.

In one embodiment, R¹ is

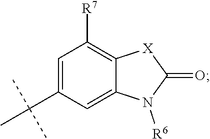

X is O; R⁶ is C₁₋₃alkyl; and R⁷ is hydrogen.

In another embodiment, R¹ is

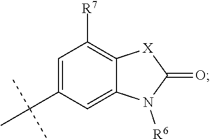

X is O; R⁶ is C₁₋₃alkyl, wherein the C₁₋₃ alkyl is optionally substituted by 1, 2 or 3 F; and R⁷ is hydrogen.

In one embodiment, R² is hydrogen, F, Cl, Br, OSO₂C₁₋₃alkyl or C₁₋₃alkyl.

In a further embodiment, R² is hydrogen, F, Cl or C₁₋₃alkyl.

In still a further embodiment, R² is hydrogen, F or C₁₋₃alkyl.

In one embodiment, R³ is hydrogen, F, Cl, Br, CN, CF₃, SO₂C₁₋₃alkyl CONH₂ or SO₂NR⁴R⁵, wherein R⁴ and R⁵ together with the nitrogen atom to which they are attached form an azetidine, pyrrolidine or piperidine ring.

In a further embodiment, R³ is hydrogen, F, Cl, CN or SO₂C₁₋₃alkyl.

In still a further embodiment, R³ is hydrogen, F or CN.

In one embodiment, R⁶ is C₁₋₃alkyl, wherein said C₁₋₃alkyl is optionally substituted by 1, 2 or 3 F and optionally by one substituent selected from the group consisting of OH, OC₁₋₃alkyl, N(C₁₋₃alkyl)₂, cyclopropyl, and tetrahydropyran.

In a further embodiment, R⁶ is C₁₋₃alkyl, wherein said C₁₋₃alkyl is optionally substituted by 1, 2 or 3 F. In still a further embodiment, R⁶ is methyl or ethyl. In still a further embodiment, R⁶ is methyl.

In one embodiment, R⁷ is hydrogen, F, Cl or CH₃. In a further embodiment R⁷ is hydrogen.

In one embodiment of the methods provided herein, the composition administered to the patient comprises an effective amount of (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide (referred to herein by its international nonproprietary name (INN) brensocatib):

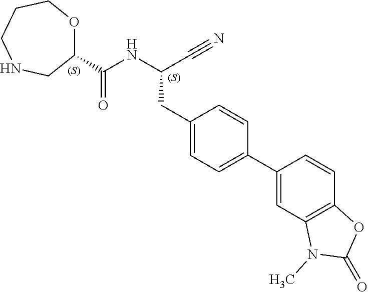

or a pharmaceutically acceptable salt thereof.

In one embodiment, the compound of Formula (I) is:
(2S)—N-[(1S)-1-Cyano-2-(4′-cyanobiphenyl-4-yl)ethyl]-1,4-oxazepane-2-carboxamide,
(2S)—N-{(1S)-1-Cyano-2-[4-(3-methyl-2-oxo-2,3-dihydro-1,3-benzoxazol-5-yl)phenyl]ethyl}-1,4-oxazepane-2-carboxamide,
(2S)—N-{(1S)-1-Cyano-2-[4-(3,7-dimethyl-2-oxo-2,3-dihydro-1,3-benzoxazol-5-yl)phenyl]ethyl}-1,4-oxazepane-2-carboxamide,
4′-[(2S)-2-Cyano-2-{[(2S)-1,4-oxazepan-2-ylcarbonyl]amino}ethyl]biphenyl-3-yl methanesulfonate,
(2S)—N-{(1S)-1-Cyano-2-[4-(3-methyl-1,2-benzoxazol-5-yl)phenyl]ethyl}-1,4-oxazepane-2-carboxamide,
(2S)—N-{(1S)-1-Cyano-2-[4′-(trifluoromethyl)biphenyl-4-yl]ethyl}-1,4-oxazepane-2-carboxamide,
(2S)—N-[(1S)-1-Cyano-2-(3′,4′-difluorobiphenyl-4-yl)ethyl]-1,4-oxazepane-2-carboxamide,
(2S)—N-{(1S)-1-Cyano-2-[4-(6-cyanopyridin-3-yl)phenyl]ethyl}-1,4-oxazepane-2-carboxamide,
(2S)—N-{(1S)-1-Cyano-2-[4-(4-methyl-3-oxo-3,4-dihydro-2H-1,4-benzothiazin-6-yl)phenyl]ethyl}-1,4-oxazepane-2-carboxamide,
(2S)—N-{(1S)-1-Cyano-2-[4-(3-ethyl-7-methyl-2-oxo-2,3-dihydro-1,3-benzoxazol-5-yl)phenyl]ethyl}-1,4-oxazepane-2-carboxamide,
(2S)—N-[(1S)-1-Cyano-2-{4-[3-(2-hydroxy-2-methylpropyl)-2-oxo-2,3-dihydro-1,3-benzoxazol-5-yl]phenyl}ethyl]-1,4-oxazepane-2-carboxamide,
(2S)—N-[(1S)-1-Cyano-2-{4-[3-(2,2-difluoroethyl)-7-fluoro-2-oxo-2,3-dihydro-1,3-benzoxazol-5-yl]phenyl}ethyl]-1,4-oxazepane-2-carboxamide,
(2S)—N-[(1S)-1-Cyano-2-(4-{3-[2-(dimethylamino)ethyl]-2-oxo-2,3-dihydro-1,3-benzoxazol-5-yl}phenyl)ethyl]-1,4-oxazepane-2-carboxamide,
(2S)—N-{(1S)-1-Cyano-2-[4-(3,3-difluoro-1-methyl-2-oxo-2,3-dihydro-1H-indol-6-yl)phenyl]ethyl}-1,4-oxazepane-2-carboxamide,
(2S)—N-{(1S)-1-Cyano-2-[4-(7-fluoro-3-methyl-2-oxo-2,3-dihydro-1,3-benzoxazol-5-yl)phenyl]ethyl}-1,4-oxazepane-2-carboxamide,
(2S)—N-{(1S)-1-Cyano-2-[4-(3-ethyl-2-oxo-2,3-dihydro-1,3-benzoxazol-5-yl)phenyl]ethyl}-1,4-oxazepane-2-carboxamide,
(2S)—N-[(1S)-1-Cyano-2-{4-[3-(cyclopropylmethyl)-2-oxo-2,3-dihydro-1,3-benzoxazol-5-yl]phenyl}ethyl]-1,4-oxazepane-2-carboxamide,
(2S)—N-[(1S)-1-Cyano-2-{4-[3-(2-methoxyethyl)-2-oxo-2,3-dihydro-1,3-benzothiazol-5-yl]phenyl}ethyl]-1,4-oxazepane-2-carboxamide,
(2S)—N-[(1S)-1-Cyano-2-{4-[2-oxo-3-(propan-2-yl)-2,3-dihydro-1,3-benzoxazol-5-yl]phenyl}ethyl]-1,4-oxazepane-2-carboxamide,
(2S)—N-{(15)-1-Cyano-2-[4-(4-methyl-3-oxo-3,4-dihydro-2H-1,4-benzoxazin-6-yl)phenyl]ethyl}-1,4-oxazepane-2-carboxamide,
(2S)—N-[(1S)-1-Cyano-2-{4-[3-(2-methoxyethyl)-2-oxo-2,3-dihydro-1,3-benzoxazol-5-yl]phenyl}ethyl]-1,4-oxazepane-2-carboxamide,
(2S)—N-{(15)-1-Cyano-2-[4-(5-cyanothiophen-2-yl)phenyl]ethyl}-1,4-oxazepane-2-carboxamide,
(2S)—N-[(1S)-2-(4′-Carbamoyl-3′-fluorobiphenyl-4-yl)-1-cyanoethyl]-1,4-oxazepane-2-carboxamide,
(2S)—N-{(15)-1-Cyano-2-[4-(1-methyl-2-oxo-1,2-dihydroquinolin-7-yl)phenyl]ethyl}-1,4-oxazepane-2-carboxamide,
(2S)—N-[(1S)-1-Cyano-2-{4-[2-oxo-3-(tetrahydro-2H-pyran-4-ylmethyl)-2,3-dihydro-1,3-benzoxazol-5-yl]phenyl}ethyl]-1,4-oxazepane-2-carboxamide,
(2S)—N-{(1S)-2-[4-(7-Chloro-3-methyl-2-oxo-2,3-dihydro-1,3-benzoxazol-5-yl)phenyl]-1-cyanoethyl}-1,4-oxazepane-2-carboxamide,
(2S)—N-[(1S)-1-Cyano-2-{4-[3-(2,2-difluoroethyl)-2-oxo-2,3-dihydro-1,3-benzoxazol-5-yl]phenyl}ethyl]-1,4-oxazepane-2-carboxamide,
(2S)—N-[(1S)-1-Cyano-2-{4-[2-oxo-3-(2,2,2-trifluoroethyl)-2,3-dihydro-1,3-benzoxazol-5-yl]phenyl}ethyl]-1,4-oxazepane-2-carboxamide,
(2S)—N-{(1S)-1-Cyano-2-[4-(3-methyl-2-oxo-2,3-dihydro-1,3-benzothiazol-5-yl)phenyl]ethyl}-1,4-oxazepane-2-carboxamide,
(2S)—N-{(1S)-1-Cyano-2-[4′-(methylsulfonyl)biphenyl-4-yl]ethyl}-1,4-oxazepane-2-carboxamide,
(2S)—N-{(1S)-2-[4′-(Azetidin-1-ylsulfonyl)biphenyl-4-yl]-1-cyanoethyl}-1,4-oxazepane-2-carboxamide,
(2S)—N-[(1S)-1-Cyano-2-(4′-fluorobiphenyl-4-yl)ethyl]-1,4-oxazepane-2-carboxamide,
(2S)—N-{(1S)-2-[4-(1,3-Benzothiazol-5-yl)phenyl]-1-cyanoethyl}-1,4-oxazepane-2-carboxamide, or
(2S)—N-[(1S)-1-Cyano-2-(4′-cyanobiphenyl-4-yl)ethyl]-1,4-oxazepane-2-carboxamide, or a pharmaceutically acceptable salt of one of the foregoing compounds.

It is also to be understood that certain compounds of Formula (I) may exist in solvated form, e.g., hydrates, including solvates of a pharmaceutically acceptable salt of a compound of Formula (I).

In one embodiment, the compound of Formula (I) provided in the methods described herein is a hydrate. In a further embodiment, the compound is a hydrate of brensocatib.

In one embodiment, a compound of Formula (I) is a racemate, a racemic mixture, a single enantiomer, an individual diastereomer or a diastereomeric mixture. It is to be understood that the present disclosure encompasses all such isomeric forms, e.g., the S,S diastereomer, the S,R diastereomer, the R,S diastereomer, and the R,R diastereomer disclosed herein, as well as a mixture of any two or more of the foregoing diastereomers.

Accordingly, in one embodiment, the compound of Formula (I) is (2S)—N-{(1S)-1-Cyano-2-[4-(3-methyl-2-oxo-2,3-dihydro-1,3-benzoxazol-5-yl)phenyl]ethyl}-1,4-oxazepane-2-carboxamide (i.e., brensocatib, the S,S isomer), shown below,

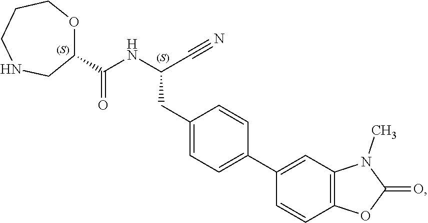

or a pharmaceutically acceptable salt thereof. In one embodiment, brensocatib is in the form of a hydrate.

In one embodiment, the compound of Formula (I) is (2R)—N-{(1R)-1-Cyano-2-[4-(3-methyl-2-oxo-2,3-dihydro-1,3-benzoxazol-5-yl)phenyl]ethyl}-1,4-oxazepane-2-carboxamide (i.e., the R,R isomer),

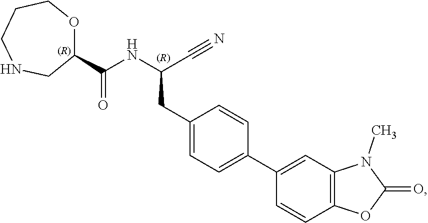

or a pharmaceutically acceptable salt thereof.

In one embodiment, the compound of Formula (I) is (2S)—N-{(1R)-1-Cyano-2-[4-(3-methyl-2-oxo-2,3-dihydro-1,3-benzoxazol-5-yl)phenyl]ethyl}-1,4-oxazepane-2-carboxamide (i.e., the S,R isomer),

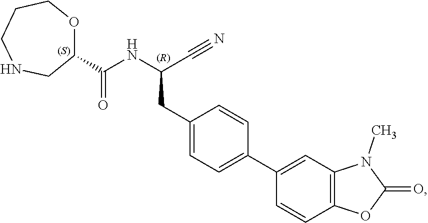

or a pharmaceutically acceptable salt thereof.

In one embodiment, the compound of Formula (I) is (2R)—N-{(1S)-1-Cyano-2-[4-(3-methyl-2-oxo-2,3-dihydro-1,3-benzoxazol-5-yl)phenyl]ethyl}-1,4-oxazepane-2-carboxamide (the R,S isomer),

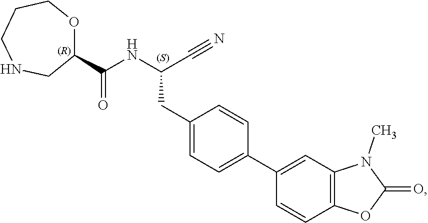

or a pharmaceutically acceptable salt thereof.

In one embodiment, the composition comprises a mixture of one or more of the aforementioned stereoisomers. The mixture in one embodiment, comprises a mixture of the the S,S isomer (brensocatib) and the S,R isomer of brensocatib. In another embodiment, the composition comprises a mixture of the S,S isomer (brensocatib) and the R,S isomer. In yet another embodiment, the composition comprises a mixture of the S,S isomer (brensocatib) and the R,R isomer.

Certain compounds of Formula (I) may also contain linkages (e.g., carbon-carbon bonds, carbon-nitrogen bonds such as amide bonds) wherein bond rotation is restricted about that particular linkage, e.g., restriction resulting from the presence of a ring bond or double bond. Accordingly, it is to be understood that the present disclosure encompasses all such isomers. Certain compound of Formula (I) may also contain multiple tautomeric forms. It is to be understood that the present disclosure encompasses all such tautomeric forms. Stereoisomers may be separated using conventional techniques, e.g., chromatography or fractional crystallization, or the stereoisomers may be made by stereoselective synthesis.

In a further embodiment, the compounds of Formula (I) encompass any isotopically-labeled (or “radio-labelled”) derivatives of a compound of Formula (I). Such a derivative is a derivative of a compound of Formula (I) wherein one or more atoms are replaced by an atom having an atomic mass or mass number different from the atomic mass or mass number typically found in nature. Examples of radionuclides that may be incorporated include ²H (also written as “D” for deuterium). As such, in one embodiment, a compound of Formula (I) is provided where one or more hydrogen atoms are replaced by one or more deuterium atoms; and the deuterated compound is used in one of the methods provided herein.

In a further embodiment, a compound of Formula (I) is administered in the form of a prodrug which is broken down in the human or animal body to ultimately provide a compound of the Formula (I). Examples of prodrugs include in vivo hydrolysable esters of a compound of the Formula (I).

An in vivo hydrolysable (or cleavable) ester of a compound of the Formula (I) that contains a carboxy or a hydroxy group is, for example, a pharmaceutically acceptable ester which is hydrolyzed in the human or animal body to produce the parent acid or alcohol. For examples of ester prodrugs derivatives, see, e.g., Curr. Drug. Metab. 2003, 4, 461, incorporated by reference herein in its entirety for all purposes.

Various other forms of prodrugs are known in the art, and can be used in the methods provided herein. For examples of prodrug derivatives, see, e.g., Nature Reviews Drug Discovery 2008, 7, 255, the disclosure of which is incorporated by reference herein in its entirety for all purposes.

The methods provided herein comprise the administration of a composition comprising an effective amount of a compound of Formula (I), or a pharmaceutically acceptable salt thereof, to a patient in need of treatment of hidradenitis suppurativa. The compounds of Formula (I) and their pharmaceutically acceptable salts are inhibitors of dipeptidyl peptidase 1 (DPP1) activity. In one embodiment, the compound is brensocatib, or a pharmaceutically acceptable salt thereof. In a further embodiment, the compound is brensocatib.

In some embodiments, the subject exhibits one or more symptoms of hidradenitis suppurativa (HS). In some embodiments, the administration of a pharmaceutical composition comprising an effective amount of any one of the compounds disclosed herein treats one or more symptoms of HS, e.g., by reducing the severity of the one or more symptoms. In some embodiments, the administration of the pharmaceutical composition reduces, diminishes the severity of, delays the onset of, or eliminates one or more symptoms of HS. In some embodiments, the one or more HS symptoms are one or more of the following: (a) one or more skin lesions; (b) one or more skin abscesses; (c) one or more fistulas; (d) one or more sinus tracts; (e) skin scarring; (f) anxiety; (g) depression; (h) suicidal thoughts; or (i) any combinations thereof. In one embodiment, the one or more symptoms of HS are one or more skin lesions. In one embodiment, the one or more symptoms of HS are one or more skin abscesses. In one embodiment, the one or more symptoms of HS are one or more fistulas. In a further embodiment, the one or more fistulas are one or more draining fistulas. In one embodiment, the one or more symptoms of HS are one or more sinus tracts. In one embodiment, the one or more HS symptoms comprises skin scarring. In one embodiment, the one or more HS symptoms comprises anxiety. In one embodiment, the one or more HS symptoms comprises depression. In one embodiment, the one or more HS symptoms comprises suicidal thoughts. In some embodiments, the one or more skin lesions, one or more skin abscesses, one or more fistulas, one or more sinus tracts, or a combination thereof are painful, inflamed, swollen, or any combination thereof, prior to the administration period. In some embodiments, during the administration period, or subsequent to the administration period, the one or more skin lesions, one or more skin abscesses, one or more fistulas, and/or one or more sinus tracts are reduced in size, and/or the inflammation, swelling and/or pain associated with the one or more skin lesions, one or more skin abscesses, one or more fistulas, and/or one or more sinus tracts is reduced.

“Prior to the administration period”, as used herein, refers to a time period of from about 28 days prior to the initial administration of the pharmaceutical composition or compound of Formula (I) provided herein, to immediately prior to the initial administration of the pharmaceutical composition or compound of Formula (I). “Immediately prior to the administration period” in one embodiment, is from about 24 hours prior to about 1 minute prior to the initial administration of the pharmaceutical composition or compound of Formula (I) provided herein.

In one embodiment, prior to the administration period is from about 28 days prior to immediately prior to the administration period. In another embodiment, prior to the administration period is from about 21 days prior to immediately prior to the administration period. In another embodiment, prior to the administration period is from about 14 days prior to immediately prior to the administration period. In even another embodiment, prior to the administration period is from about 10 days prior to immediately prior to the administration period. In yet even another embodiment, prior to the administration period is from about 7 days prior to immediately prior to the administration period. In even yet another embodiment, prior to the administration period is from about 4 days prior to immediately prior to the administration period.

In one embodiment, “prior to the administration period” is from about 28 days prior to about 1 day prior to the administration period. In another embodiment, prior to the administration period is from about 21 days prior to about 1 day prior to the administration period. In another embodiment, prior to the administration period is from about 14 days prior to about 1 day prior to the administration period. In even another embodiment, prior to the administration period is from about 10 days prior to about 1 day prior to the administration period. In yet even another embodiment, prior to the administration period is from about 7 days prior to about 1 day prior to the administration period. In even yet another embodiment, prior to the administration period is from about 4 days prior to about 1 day prior to the administration period.

The one or more skin lesions, one or more skin abscesses, one or more fistulas, one or more sinus tracts, or a combination thereof may be found in any part of the body of the subject, for example, a part of the body where skin tissue rubs against other skin tissue. In some embodiments, the one or more skin lesions, one or more skin abscesses, one or more fistulas, one or more sinus tracts, or a combination thereof are present on one or both the armpits, the groin, the buttocks, or a combination thereof, of the subject. In some embodiments, the one or more skin lesions, one or more skin abscesses, one or more fistulas, one or more sinus tracts, or a combination thereof are associated with hair follicles. In some embodiments, the one or more skin lesions are one or more skin nodules. In some embodiments, the one or more skin nodules are one or more inflammatory skin nodules. In some embodiments, the one or more skin lesions are interconnected by one or more sinus tracts. In some embodiments, the one or more skin lesions are one or more fistulas. In a further embodiment, the one or more fistulas are one or more draining fistulas.

In some embodiments, a method of treating HS comprises reducing a skin abscess and inflammatory skin nodule count (AN count) of the subject during or subsequent to the administration period, as compared to an AN count of the subject prior to the administration period. As used herein, the “AN count” of the subject comprises a count of the total number of skin abscesses and inflammatory skin nodules on the subject. Further details on the AN count are provided in Kimball et al. Journal of the European Academy of Dermatology and Venereology 2016, 30, 989-994, the contents of which are herein incorporated by reference in its entirety. In some embodiments, the AN count of the subject prior to the administration period is 3 or more. For example, in one embodiment, the AN count of the subject prior to the administration period is 3, 4, 5, 6, 7, 8, 9, 10, 11, 12, 13, 14, 15, or greater than 15.

In some embodiments, the AN count of the subject during or subsequent to the administration period is less than the AN count of the subject prior to the administration period. In some embodiments, the AN count of the subject during or subsequent to the administration period is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, including all values and subranges that lie therebetween) less than the AN count of the subject prior to the administration period. In some embodiments, the AN count of the subject during or subsequent to the administration period is at least 10% less than the AN count of the subject prior to the administration period. In some embodiments, the AN count of the subject during or subsequent to the administration period is at least 50% less than the AN count of the subject prior to the administration period.

In one embodiment, reducing the AN count comprises reducing the AN count by about 1, about 2, about 3, about 4, about 5, about 6, about 7, about 8, about 9 or about 10. In another embodiment, reducing the AN count comprises reducing the AN count by at least about 1, at least about 2, at least about 3, at least about 4, at least about 5, at least about 6, at least about 7, at least about 8, at least about 9 or at least about 10.

In one embodiment, the method of the invention comprises reducing an occurrence of HS flares in the subject during or subsequent to the administration period, as compared to an occurrence of HS flares in the subject prior to the administration period. As used herein, “HS flare” refers to an increase of at least 25% in AN count with an absolute increase in AN count of ≥2, as compared to a baseline AN count.

In some embodiments, the methods comprise reducing or maintaining a fistula count (e.g., draining fistula count) of the subject during or subsequent to the administration period, as compared to a fistula count (e.g., draining fistula count) of the subject prior to the administration period. As used herein, the fistula count of the subject is a count of the total number of fistulas on the subject. For instance, the draining fistula count of the subject is a count of the total number of draining fistulas in the subject. Further details on fistula count are provided in Kimball et al. Journal of the European Academy of Dermatology and Venereology 2016, 30, 989-994, the contents of which are herein incorporated by reference in its entirety. In some embodiments, the fistula count (e.g., draining fistula count) of the subject prior to the administration period is ≤20. In some embodiments, the fistula count (e.g., draining fistula count) of the subject prior to the administration period is 20, 19, 18, 17, 16, 15, 14, 13, 12, 11, 10, 9, 8, 7, 6, 5, 4, 3, 2, 1, or 0.

In some embodiments, the method comprises maintaining or reducing the fistula count (e.g., draining fistula count) of the subject during or subsequent to the administration period compared to the fistula count (e.g., draining fistula count) of the subject prior to the administration period. In some embodiments, the fistula count (e.g., draining fistula count) of the subject during or subsequent to the administration period is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, including all values and subranges that lie therebetween) less than the fistula count (e.g., draining fistula count) of the subject prior to the administration period.

In one embodiment, reducing the fistula count comprises reducing the fistula count by about 1, about 2, about 3, about 4, about 5, about 6, about 7, about 8, about 9, about 10, about 11, about 12, about 13, about 14 or about 15. In another embodiment, reducing the fistula count comprises reducing the fistula count by at least about 1, at least about 2, at least about 3, at least about 4, at least about 5, at least about 6, at least about 7, at least about 8, at least about 9, at least about 10, at least about 11, at least about 12, at least about 13, at least about 14 or at least about 15. In a further embodiment, the fistula count is a draining fistula count.

In some embodiments of the methods for treating HS, the subject achieves a Hidradenitis Suppurativa Clinical Response (HiSCR) during or subsequent to the administration period. As used herein, a subject is said to have achieved HiSCRn if the subject exhibits at least a n % reduction in total AN count with no increase in skin abscess count and no increase in draining fistula count during or subsequent to the administration period, as compared to a total AN count, a skin abscess count, and a draining fistula count of the subject, respectively, prior to the administration period. For example, a subject is said to have achieved: “HiSCR50”, “HiSCR75” or “HiSCR90” if the subject exhibits at least a 50%, 75% or 90% reduction, respectively, in the total AN count with no increase in skin abscess count and no increase in draining fistula count during or subsequent to the administration period, as compared to a total AN count, a skin abscess count, and a draining fistula count of the subject, respectively, prior to the administration period. Further details of evaluating HiSCR are provided in Kimball et al. Journal of the European Academy of Dermatology and Venereology 2016, 30, 989-994, the contents of which are herein incorporated by reference in its entirety.

In some embodiments of the methods for treating HS, the subject achieves a modified Hidradenitis Suppurativa Clinical Response (mHiSCR) during or subsequent to the administration period. mHiSCR is defined as a ≥50% reduction in total number of inflammatory nodules, abscesses, and draining fistulas and a ≥50% reduction in total number of draining fistulas, as compared to a total number of inflammatory nodules, abscesses, and draining fistulas and a total number of draining fistulas of the subject, respectively, prior to the administration period.

In some embodiments, a method of treating HS provided herein comprises reducing a subject's Patient's Global Assessment of Skin Pain (PGA-SP) score during or subsequent to the administration period, as compared to a PGA-SP score of the subject prior to the administration period. As used herein, the PGA-SP score refers to a score that the patient assigns to assess skin pain “at its worst” in the past 24-hour period, using an 11-point numeric rating scale (i.e., NRS for skin pain) in which 0 is no skin pain and 10 is skin pain as bad as can be imagined. Further details on PGA-SP are provided in Kimball et al., J Am Acad Dermatol, 2018, the contents of which are herein incorporated by reference in its entirety. In some embodiments, the PGA-SP score of the subject prior to the administration period is ≥3. In some embodiments, the PGA-SP score of the subject prior to the administration period is 3, 4, 5, 6, 7, 8, 9, or 10.

In one embodiment, reducing the PGA-SP score comprises reducing the PGA-SP score by about 1, about 2, about 3, about 4, about 5, about 6, about 7, about 8, or about 9. In another embodiment, reducing the PGA-SP score comprises reducing the PGA-SP score by at least about 1, at least about 2, at least about 3, at least about 4, at least about 5, at least about 6, at least about 7, at least about 8, or at least about 9.

In some embodiments, the PGA-SP score of the subject during or subsequent to the administration period is reduced to 2, 1 or 0.

Reducing the PGA-SP score of the subject during or subsequent to the administration period, in one embodiment, comprises reducing the PGA-SP score by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, including all values and subranges that lie therebetween) as compared to the PGA-SP score of the subject prior to the administration period. In some embodiments, reducing the PGA-SP score of the subject during or subsequent to the administration period comprises reducing by at least about 10%, as compared to the PGA-SP score of the subject prior to the administration period. In some embodiments, the PGA-SP score of the subject during or subsequent to the administration period is reduced to 3 or less.

In some embodiments, the method of treating HS comprises reducing an International Hidradenitis Suppurativa Severity (IHS4) score of the subject during or subsequent to the administration period, as compared to an IHS4 score of the subject prior to the administration period. As used herein, the IHS4 refers to the number of inflammatory nodules (multiplied by 1) plus the number of abscesses (multiplied by 2) plus the number of draining fistulas (multiplied by 4). A total IHS4 score of 3 or less signifies mild (Stage I HS), 4-10 signifies moderate (Stage II HS) and 11 or higher signifies severe disease (Stage III HS). Further details on measuring IHS4 are provided in Zouboulis et al. British Journal of Dermatology (2017) 177, pp 1401-1409, the contents of which are herein incorporated by reference in its entirety.

In one embodiment of the methods for treating HS, the IHS4 score of the subject is ≥4 prior to the administration period. In a further embodiment, the IHS4 score of the subject is ≥4 and ≤10 prior to the administration period. In another embodiment, the IHS4 score of the subject is ≥11 prior to the administration period.

In some embodiments, a method of treating HS comprises reducing the IHS4 score of the subject during or subsequent to the administration period by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, including all values and subranges that lie therebetween) compared to the IHS4 score of the subject prior to the administration period. In some embodiments, a method of treating HS comprises reducing the IHS4 score of the subject during or subsequent to the administration period by at least about 10% compared to the IHS4 score of the subject prior to the administration period. In some embodiments, a method of treating HS comprises reducing the IHS4 score of the subject during or subsequent to the administration period to 0, 1, 2, 3, 4, 5, 6, 7, 8, 9, or 10.

In one embodiment, reducing the IHS4 score comprises reducing the IHS4 score by about 1, about 2, about 3, about 4, about 5, about 6, about 7, about 8, or about 9. In another embodiment, reducing the IHS4 score comprises reducing the IHS4 score by at least about 1, at least about 2, at least about 3, at least about 4, at least about 5, at least about 6, at least about 7, at least about 8, or at least about 9.

In one embodiment, the method of treating HS comprises reducing an IHS4 score of the subject during or subsequent to the administration period from an HS Hurley Stage III score to an HS Hurley Stage II score.

In another embodiment, the method of treating HS comprises reducing an IHS4 score of the subject during or subsequent to the administration period from an HS Hurley Stage III score to an HS Hurley Stage I score.

In yet another embodiment, the method of treating HS comprises reducing an IHS4 score of the subject during or subsequent to the administration period from an HS Hurley Stage II score to an HS Hurley Stage I score.

In some embodiments, a method of treating HS according to the invention comprises improving the Hurley Stage of the HS of the subject during or subsequent to the administration period, as compared to the Hurley Stage of the HS of the subject prior to the administration period. The Hurley Stage is a severity classification for HS that was developed in 1989 and is widely used for the determination of HS severity (Hurley, Dermatologic surgery. New York: Marcel Dekker. 1989; 729:39; Ovadja et al., Br J Dermatol. 2019; 181(2):344-49, each of which is incorporated herein by reference in its entirety). In one embodiment, the improving the Hurley Stage of the HS of the subject comprises reducing the severity of the HS of the subject from Hurley Stage III to Hurley Stage II. In another embodiment, the improving the Hurley Stage of the HS of the subject comprises reducing the severity of the HS of the subject from Hurley Stage III to Hurley Stage I. In another embodiment, the improving the Hurley Stage of the HS of the subject comprises reducing the severity of the HS of the subject from Hurley Stage II to Hurley Stage I.

In some embodiments, a method of treating HS according to the invention comprises reducing a Hidradenitis Suppurativa Quality of Life (HiSQOL) score of the subject during or subsequent to the administration period, as compared to a HiSQOL score of the subject prior to the administration period. As used herein, the HiSQOL refers to a measure of health-related quality of life (HRQOL) that is specific to HS, developed by HIdradenitis SuppuraTiva cORe outcomes set International Collaboration (HISTORIC). The 17-item HiSQOL includes four symptom items, eight activity-adaptation items, and five psychosocial items. The item scores are summed to create a total ranging from 0 to 68, with higher scores indicating more severe impact on HRQOL and more severe disease. Further details on measuring HiSQOL are provided in Kirby et al., The Hidradenitis Suppurativa Quality of Life (HiSQOL) score: development and validation of a measure for clinical trials. Br J Dermatol. 2020 August; 183(2):340-348, the contents of which are herein incorporated by reference in its entirety for all purposes. In some embodiments, the method of treating HS comprises reducing the HiSQOL score of the subject during or subsequent to the administration period by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, including all values and subranges that lie therebetween) compared to the HiSQOL score of the subject prior to the administration period. In one embodiment, reducing the HiSQOL score comprises reducing the HiSQOL score by about 1, about 5, about 10, about 15, about 20, about 25, about 30, about 35, about 40, about 45, about 50, about 55, about 60, or about 65. In another embodiment, reducing the HiSQOL score comprises reducing the HiSQOL score by at least about 1, at least about 5, at least about 10, at least about 15, at least about 20, at least about 25, at least about 30, at least about 35, at least about 40, at least about 45, at least about 50, at least about 55, at least about 60, or at least about 65.

In some embodiments, a method of treating HS according to the invention comprises reducing a Hidradenitis Suppurativa—Investigator's Global Assessment (HS-IGA) score of the subject during or subsequent to the administration period, as compared to an HS-IGA score of the subject prior to the administration period. HS-IGA is a clinical endpoint for measuring disease activity and response to treatment in patients with HS that is based on the maximum count of abscesses, fistulas (draining and nondraining), and nodules (inflammatory and noninflammatory), measured separately for the upper body and the lower body regions (Garg et al., Br J Dermatol. 2022; 187(2):203-210, incorporated herein by reference in its entirety). HS-IGA is scored as a number between 0 and 5 based on the maximum lesion count for either the upper body or the lower body regions, whichever is greater. A response to treatment is defined as a 2-point reduction (improvement) in HS-IGA score relative to baseline. In some embodiments, the method of treating HS comprises reducing the HS-IGA score of the subject during or subsequent to the administration period by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, including all values and subranges that lie therebetween) compared to the HS-IGA score of the subject prior to the administration period. In one embodiment, reducing the HS-IGA score comprises reducing the HS-IGA score by about 1, about 2, about 3, about 4, or about 5. In another embodiment, reducing the HS-IGA score comprises reducing the HS-IGA score by at least about 1, at least about 2, at least about 3, at least about 4, or at least about 5. In a further embodiment, reducing the HS-IGA score comprises reducing the HS-IGA score by at least 2 points.

In some embodiments, a method of treating HS according to the invention comprises reducing a Dermatology Life Quality Index (DLQI) score of the subject during or subsequent to the administration period, as compared to a DLQI score of the subject prior to the administration period. DLQI is a self-administered questionnaire designed to measure the health related quality of life of adults (e.g., >16 years of age) suffering from a skin disease (Finlay et al., Clin Exp Dermatol. 1994; 19(3):210-6, incorporated herein by reference in its entirety). It is comprised of 10 questions concerning the rater's perception of the impact of skin diseases on different aspects of the rater's quality of life over the past week. Each question has 4 alternative responses of not at all, a little, a lot, or very much, with corresponding scores of 0, 1, 2, and 3, respectively (a response of not relevant is scored as 0). The DLQI is the sum of scores with a range of 0 to 30. The higher the score, the greater the impairment in quality of life: 0-1=no effect at all; 2-5=small effect; 6-10=moderate effect; 11-20=very large effect; and 21-30=extremely large effect. In some embodiments, the method of treating HS comprises reducing the DLQI score of the subject during or subsequent to the administration period by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, including all values and subranges that lie therebetween) compared to the DLQI score of the subject prior to the administration period. In one embodiment, reducing the DLQI score comprises reducing the DLQI score by about 1, about 3, about 5, about 10, about 15, about 20, about 25, about 28, or about 30. In another embodiment, reducing the DLQI score comprises reducing the DLQI score by at least about 1, at least about 3, at least about 5, at least about 10, at least about 15, at least about 20, at least about 25, at least about 28, or at least about 30.

In some embodiments, a method of treating HS according to the invention comprises reducing a HS-Related Patient Global Assessment (HS-related PtGA) score of the subject during or subsequent to the administration period, as compared to an HS-related PtGA score of the subject prior to the administration period. HS-related PtGA is a single-item questionnaire assessing how much HS has influenced the participant's quality of life over the previous 7 days with 5 response levels of not at all, slightly, moderately, very much, and extremely and corresponding scores of 0, 1, 2, 3, and 4, respectively (Kirby et al., Br J Dermatol. 2021; 184(4):681-87, incorporated herein by reference in its entirety). In some embodiments, the method of treating HS comprises reducing the HS-related PtGA score of the subject during or subsequent to the administration period by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, including all values and subranges that lie therebetween) compared to the HS-related PtGA score of the subject prior to the administration period. In one embodiment, reducing the HS-related PtGA score comprises reducing the HS-related PtGA score by about 1, about 2, about 3, or about 4. In another embodiment, reducing the HS-related PtGA score comprises reducing the HS-related PtGA score by at least about 1, at least about 2, at least about 3, or at least about 4.

In some embodiments, the number of neutrophils in a biological sample obtained from the subject prior to the administration period is greater compared to a control subject who does not have HS. In some embodiments, the number of neutrophils in a biological sample obtained from the subject prior to the administration period is at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 25 fold, or about 30 fold, including all values and subranges that lie therebetween) greater than a control subject who does not have HS. In some embodiments, the number of neutrophils in a biological sample obtained from the subject prior to the administration period is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween) greater than a control subject who does not have HS.

In some embodiments, a method of treating HS provided herein comprises reducing the number of neutrophils in a biological sample obtained from the subject during or subsequent to the administration period compared to (a) the number of neutrophils in a counterpart biological sample obtained from the subject prior to the administration period, and/or (b) the number of neutrophils in a counterpart biological sample obtained from a control subject, wherein the control subject has HS and is not administered the composition. For example, in some embodiments, the number of neutrophils in a biological sample obtained from the subject during or subsequent to the administration period is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, including all values and subranges that lie therebetween) less than: (a) the number of neutrophils in a counterpart biological sample obtained from the subject prior to the administration period, and/or (b) the number of neutrophils in a counterpart biological sample obtained from a control subject, wherein the control subject has HS and is not administered the composition.

In some embodiments, the level of SA100A7 (S100 calcium-binding protein A7, also known as psoriasin) in a biological sample obtained from the subject prior to the administration period is increased compared to a control subject who does not have HS. The “level of SA100A7” may refer to the level of protein expression of the SA1007 protein, or the level of expression of a gene encoding SA1007. In some embodiments, the level of SA100A7 in a biological sample obtained from the subject prior to the administration period is at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 25 fold, or about 30 fold, including all values and subranges that lie therebetween) greater than a control subject who does not have HS. In some embodiments, the level of SA100A7 in a biological sample obtained from the subject prior to the administration period is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween) greater than a control subject who does not have HS.

In some embodiments, a method of treating HS comprises decreasing the level of SA100A7 in a biological sample obtained from the subject during or subsequent to the administration period compared to (a) the level of SA100A7 in a counterpart biological sample obtained from the subject prior to the administration period, and/or (b) the level of SA100A7 in a counterpart biological sample obtained from a control subject, wherein the control subject has HS and is not administered the composition comprising a compound of Formula (I). In some embodiments, the level of SA100A7 in a biological sample obtained from the subject during or subsequent to the administration period is decreased by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, including all values and subranges that lie therebetween) compared to: (a) the level of SA100A7 in a counterpart biological sample obtained from the subject prior to the administration period, and/or (b) the level of SA100A7 in a counterpart biological sample obtained from a control subject, wherein the control subject has HS and is not administered the composition.

In some embodiments, the level of myeloperoxidase (MPO) in a biological sample obtained from the subject prior to the administration period is increased compared to a control subject who does not have HS. The “level of MPO” may refer to the level of protein expression of the MPO protein, or the level of expression of a gene encoding MPO. In some embodiments, the level of MPO in a biological sample obtained from the subject prior to the administration period is at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 25 fold, or about 30 fold, including all values and subranges that lie therebetween) greater than a control subject who does not have HS. In some embodiments, the level of MPO in a biological sample obtained from the subject prior to the administration period is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween) greater than a control subject who does not have HS.

In some embodiments, a method of treating HS provided herein comprises decreasing the level of MPO in a subject (as measured from a biological sample obtained from the subject) during or subsequent to the administration period compared to (a) the level of MPO of the subject prior to the administration period, and/or (b) the level of MPO in a counterpart biological sample obtained from a control subject, wherein the control subject has HS and is not administered the composition provided herein. In a further embodiment, the decreasing is by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, including all values and subranges that lie therebetween).

In some embodiments, the level of peptidylarginine deiminase 4 (PAD4) in a biological sample obtained from the subject prior to the administration period is increased compared to a control subject who does not have HS. The “level of PAD4” may refer to the level of protein expression of the PAD4 protein, or the level of expression of a gene encoding PAD4. In some embodiments, the level of PAD4 in a biological sample obtained from the subject prior to the administration period is at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 25 fold, or about 30 fold, including all values and subranges that lie therebetween) greater than a control subject who does not have HS. In some embodiments, the level of PAD4 in a biological sample obtained from the subject prior to the administration period is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween) greater than a control subject who does not have HS.

In some embodiments, a method of treating HS provided herein comprises decreasing the level of PAD4 in a subject (e.g., from a biological sample obtained from the subject) during or subsequent to the administration period compared to (a) the level of PAD4 in a counterpart biological sample obtained from the subject prior to the administration period, and/or (b) the level of PAD4 in a counterpart biological sample obtained from a control subject, wherein the control subject has HS and is not administered the composition provided herein. In a further embodiment, the decreasing is by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, including all values and subranges that lie therebetween).

Peptidylarginine deiminase 4 (PAD4) promotes protein citrullination, such as, histone citrullination. Therefore, in some embodiments, the level of citrullinated peptides in a biological sample obtained from the subject prior to the administration period is increased compared to a control subject who does not have HS. In some embodiments, the level of citrullinated peptides in a biological sample obtained from the subject prior to the administration period is at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 25 fold, or about 30 fold, including all values and subranges that lie therebetween) greater than a control subject who does not have HS. In some embodiments, the level of citrullinated peptides in a biological sample obtained from the subject prior to the administration period is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween) greater than a control subject who does not have HS.

In some embodiments, a method of treating HS provided herein comprises decreasing the level of citrullinated peptides in the subject (i.e., from a biological sample obtained from the subject) during or subsequent to the administration period compared to (a) the level of citrullinated peptides in the subject (i.e., from a counterpart biological sample obtained from the subject) prior to the administration period, and/or (b) the level of citrullinated peptides in a counterpart biological sample obtained from a control subject, wherein the control subject has HS and is not administered the composition. In a further embodiment, the decreasing is by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, including all values and subranges that lie therebetween).

In some embodiments, the level of a cytokine, a lipid mediator, or a combination thereof in a biological sample obtained from the subject prior to the administration period is increased compared to a control subject who does not have HS. In some embodiments, the level of cytokine, a lipid mediator, or a combination thereof in a biological sample obtained from the subject prior to the administration period is at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 25 fold, or about 30 fold, including all values and subranges that lie therebetween) greater than a control subject who does not have HS. In some embodiments, the level of cytokine, a lipid mediator, or a combination thereof in a biological sample obtained from the subject prior to the administration period is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween) greater than a control subject who does not have HS.

In some embodiments, a method of treating HS provided herein comprises decreasing the level of a cytokine, a lipid mediator, or a combination thereof in the subject (i.e., from a biological sample obtained from the subject) during or subsequent to the administration period compared to (a) the level of the cytokine, the lipid mediator, or a combination thereof in the subject (i.e., from a counterpart biological sample obtained from the subject) prior to the administration period, and/or (b) the level of the cytokine, the lipid mediator, or a combination thereof in a counterpart biological sample obtained from a control subject, wherein the control subject has HS and is not administered the composition provided herein. In a further embodiment, the decreasing is by at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, including all values and subranges that lie therebetween).

In some embodiments, the cytokine is a chemokine. In some embodiments, the cytokine is IL-1, IL-36, IL-8, IL-17, TNF-alpha, or any combination thereof.

In some embodiments, the level of C-reactive protein (CRP) in a biological sample obtained from the subject prior to the administration period is increased compared to a control subject who does not have HS. In some embodiments, the level of CRP in a biological sample obtained from the subject prior to the administration period is at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 25 fold, or about 30 fold, including all values and subranges that lie therebetween) greater than a control subject who does not have HS. In some embodiments, the level of CRP in a biological sample obtained from the subject prior to the administration period is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween) greater than a control subject who does not have HS.

In some embodiments, the subject in need of treatment is at a risk for developing HS. In some embodiments, the subject at a risk for developing HS is or was repeatedly exposed to tobacco smoke; has a family history of hidradenitis suppurativa; or a combination thereof.

In some embodiments, the subject in need of treatment is or was repeatedly exposed to tobacco smoke. As used herein, “repeated exposure” to tobacco smoke relates to exposure that is frequent enough to be associated with, result in, or increase the risk of developing one or more adverse effects of inhaling tobacco smoke. Non-limiting adverse effects of inhaling tobacco smoke are cancer (e.g., lung cancer), coronary heart disease, respiratory infections, stroke, lung disease, diabetes, chronic obstructive pulmonary disease (COPD), emphysema, chronic bronchitis, tuberculosis, eye diseases, immune dynsfuction, and rheumatoid arthritis. In some embodiments, the subject is exposed to tobacco smoke at least once a month, for example, once in two weeks, once a week, every alternate day, every day, or several times a day. In some embodiments, the subject is an active smoker of tobacco-containing products, such as cigarettes. In some embodiments, the subject is passively exposed to tobacco smoke.

In some embodiments, the HS is associated with the presence or development of one or more of: acne, arthritis, diabetes, metabolic syndrome, inflammatory bowel disease, obesity, or any combination thereof.

In some embodiments, the level of DPP1 in a biological sample obtained from the subject is in the range of about 1 ng/mL to about 1000 ng/mL prior to the administration period. The disclosure further provides methods of treating hidradenitis suppurativa in a subject in need thereof, comprising: (a) determining the level of DPP1 in a biological sample obtained from the subject, and (b) administering to the subject, a pharmaceutical composition comprising an effective amount of any one of the compounds disclosed herein. In some embodiments, the level of DPP1 determined in step (a), or the level of DPP1 in a biological sample obtained from the subject prior to the administration period is in the range of about 1 ng/mL to about 1000 ng/mL, for example about 3 ng/mL, about 5 ng/mL, about 7 ng/mL, about 10 ng/mL, about 13 ng/mL, about 15 ng/mL, about 17 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 150 ng/mL, about 200 ng/mL, about 250 ng/mL, about 300 ng/mL, about 350 ng/mL, about 400 ng/mL, about 450 ng/mL, about 500 ng/mL, about 550 ng/mL, about 600 ng/mL, about 650 ng/mL, about 700 ng/mL, about 750 ng/mL, about 800 ng/mL, about 850 ng/mL, about 900 ng/mL, about 950 ng/mL, or about 1000 ng/mL, including all values and subranges that lie therebetween. In some embodiments, the level of DPP1 determined in step (a), or the level of DPP1 in a biological sample obtained from the subject prior to the administration period is in the range of about 5 ng/mL to about 20 ng/mL. In some embodiments, the level of DPP1 determined in step (a), or the level of DPP1 in a biological sample obtained from the subject prior to the administration period is in the range of about 1 ng/mL to about 100 ng/mL. In some embodiments, the level of DPP1 refers to the level of total DPP1, including both active and inactive DPP1. In other embodiments, the level of DPP1 refers to the level of active DPP1.

In some embodiments, the level or activity of DPP1 determined in step (a), or the level or activity of DPP1 in a biological sample obtained from the subject prior to the administration period is at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 25 fold, or about 30 fold, including all values and subranges that lie therebetween) greater than the level or activity, respectively, of DPP1 in a healthy subject who does not have hidradenitis suppurativa.

In some embodiments, the level or activity of DPP1 determined in step (a), or the level or activity of DPP1 in a biological sample obtained from the subject prior to the administration period is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000%, including all values and subranges that lie therebetween) greater than the level or activity, respectively, of DPP1 in a healthy subject who does not have hidradenitis suppurativa.

In some embodiments, the activity of DPP1 in a biological sample obtained from the subject during or subsequent to the administration period is less than: (a) the activity of DPP1 in a counterpart biological sample obtained from the subject prior to the administration period, and/or (b) the activity of DPP1 in a counterpart biological sample obtained from a control subject, wherein the control subject has hidradenitis suppurativa and is not administered the composition. In some embodiments, the activity of DPP1 in a biological sample obtained from the subject during or subsequent to the administration period is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, including all values and subranges that lie therebetween) less than: (a) the activity of DPP1 in a counterpart biological sample obtained from the subject prior to the administration period, and/or (b) the activity of DPP1 in a counterpart biological sample obtained from a control subject, wherein the control subject has hidradenitis suppurativa and is not administered the composition.

In some embodiments, the level of DPP1 in a biological sample obtained from the subject during or subsequent to the administration period is less than: (a) the level of DPP1 in a counterpart biological sample obtained from the subject prior to the administration period, and/or (b) the level of DPP1 in a counterpart biological sample obtained from a control subject, wherein the control subject has HS and is not administered the composition. In some embodiments, the level of DPP1 in a biological sample obtained from the subject during or subsequent to the administration period is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, including all values and subranges that lie therebetween) less than: (a) the level of DPP1 in a counterpart biological sample obtained from the subject prior to the administration period, and/or (b) the level of DPP1 in a counterpart biological sample obtained from a control subject, wherein the control subject has hidradenitis suppurativa and is not administered the composition.

In some embodiments, DPP1 and its levels may be detected and/or quantified using methods, such as, for example, western blotting and enzymatic activity assays. Further details are provided in Pham et al., Proc Natl Acad Sci 96(15):8627-32 (1999); Chen et al., J. Med. Chem. 64:11857-11885 (2021); Hamon et al., J Biol Chem. 291(16):8486-99 (2016); and International Patent Application No. PCT/US2021/042199, the contents of each of which is incorporated herein by reference in its entirety.

In some embodiments, the level of neutrophil extracellular traps (NETs) in a biological sample obtained from the subject is in the range of about 1 ng/mL to about 1000 ng/mL prior to the administration period. The disclosure further provides methods of treating hidradenitis suppurativa in a subject in need thereof, comprising: (a) determining the level of NETs in a biological sample obtained from the subject, and (b) administering to the subject, a pharmaceutical composition comprising an effective amount of any one of the compounds disclosed herein. In some embodiments, the level of NETs determined in step (a), or the level of neutrophil extracellular traps (NETs) in a biological sample obtained from the subject prior to the administration period is in the range of about 1 ng/mL to about 1000 ng/mL, for example about 3 ng/mL, about 5 ng/mL, about 7 ng/mL, about 10 ng/mL, about 13 ng/mL, about 15 ng/mL, about 17 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 150 ng/mL, about 200 ng/mL, about 250 ng/mL, about 300 ng/mL, about 350 ng/mL, about 400 ng/mL, about 450 ng/mL, about 500 ng/mL, about 550 ng/mL, about 600 ng/mL, about 650 ng/mL, about 700 ng/mL, about 750 ng/mL, about 800 ng/mL, about 850 ng/mL, about 900 ng/mL, about 950 ng/mL, or about 1000 ng/mL, including all values and subranges that lie therebetween.

In some embodiments, the level of NETs determined in step (a), or the level of NETs in a biological sample obtained from the subject prior to the administration period is at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 25 fold, or about 30 fold, including all values and subranges that lie therebetween) greater than the level of NETs in a healthy subject who does not have hidradenitis suppurativa.

In some embodiments, the level of NETs determined in step (a), or the level of NETs in a biological sample obtained from the subject prior to the administration period is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000% including all values and subranges that lie therebetween) greater than the level of NETs in a healthy subject who does not have hidradenitis suppurativa.

In some embodiments, the level of neutrophil extracellular traps (NETs) in a biological sample obtained from the subject during or subsequent to the administration period is less than: (a) the level of NETs in a counterpart biological sample obtained from the subject prior to the administration period, and/or (b) the level of NETs in a counterpart biological sample obtained from a control subject, wherein the control subject has hidradenitis suppurativa and is not administered the composition. In some embodiments, the level of NETs in a biological sample obtained from the subject during or subsequent to the administration period is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, including all values and subranges that lie therebetween) less than: (a) the level of NETs in a counterpart biological sample obtained from the subject prior to the administration period, and/or (b) the level of NETs in a counterpart biological sample obtained from a control subject, wherein the control subject has hidradenitis suppurativa and is not administered the composition.

In some embodiments, the level of NETs is the level of circulating plasma NETs. In some embodiments, the level of NETs is determined by measuring DNA-complexed with NET-molecules like myeloperoxidase (MPO-DNA) or neutrophil elastase (NE-DNA) using enzyme-linked immunosorbent assays (ELISAs), measuring the presence of citrullinated histones by fluorescence microscopy, flow cytometric detection of NET-components, immunofluorescence to detect the colocalization of NET-associated molecules (NE, MPO, CitH3) with extracellular DNA or using flow cytometry or confocal microscopy-based methods. Further details on determining the levels of NETs is provided in Arends et al., J. Vis. Exp. (143), e59150, (2019); Gál, et al., Sci Rep 10, 4320 (2020); and Thålin et al., Arteriosclerosis, Thrombosis, and Vascular Biology. 39, 1724-1738 (2019), each of which is incorporated herein by reference in its entirety for all purposes.

In some embodiments, the level of a neutrophil serine protease (NSP) in a biological sample obtained from the subject is in the range of about 1 ng/mL to about 1000 ng/mL prior to the administration period. The disclosure further provides methods of treating hidradenitis suppurativa in a subject in need thereof, comprising: (a) determining the level of a NSP in a biological sample obtained from the subject, and (b) administering to the subject, a pharmaceutical composition comprising an effective amount of any one of the compounds disclosed herein. In some embodiments, the level of the NSP determined in step (a), or the level of the NSP in a biological sample obtained from the subject prior to the administration period is in the range of about 1 ng/mL to about 1000 ng/mL, for example about 3 ng/mL, about 5 ng/mL, about 7 ng/mL, about 10 ng/mL, about 13 ng/mL, about 15 ng/mL, about 17 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 150 ng/mL, about 200 ng/mL, about 250 ng/mL, about 300 ng/mL, about 350 ng/mL, about 400 ng/mL, about 450 ng/mL, about 500 ng/mL, about 550 ng/mL, about 600 ng/mL, about 650 ng/mL, about 700 ng/mL, about 750 ng/mL, about 800 ng/mL, about 850 ng/mL, about 900 ng/mL, about 950 ng/mL, or about 1000 ng/mL, including all values and subranges that lie therebetween. In the present disclosure, in some embodiments, the level of an NSP refers to the level of total NSP, including both the active and inactive forms of the NSP. In other embodiments, the level of an NSP refers to the level of the active form(s) of the NSP.

In some embodiments, the level or activity of the NSP determined in step (a), or the level or activity of the NSP in a biological sample obtained from the subject prior to the administration period is at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 25 fold, or about 30 fold, including all values and subranges that lie therebetween) greater than the level or activity, respectively, of the NSP in a healthy subject who does not have hidradenitis suppurativa.

In some embodiments, the level or activity of the NSP determined in step (a), or the level or activity of the NSP in a biological sample obtained from the subject prior to the administration period is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000% including all values and subranges that lie therebetween) greater than the level or activity, respectively of the NSP in a healthy subject who does not have HS.

In some embodiments, the activity of a neutrophil serine protease (NSP) in a biological sample obtained from the subject during or subsequent to the administration period is less than: (a) the activity of the NSP in a counterpart biological sample obtained from the subject prior to the administration period, and/or (b) the activity of the NSP in a counterpart biological sample obtained from a control subject, wherein the control subject has hidradenitis suppurativa and is not administered the composition. In some embodiments, the activity of the NSP in a biological sample obtained from the subject during or subsequent to the administration period is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, including all values and subranges that lie therebetween) less than: (a) the activity of the NSP in a counterpart biological sample obtained from the subject prior to the administration period, and/or (b) the activity of the NSP in a counterpart biological sample obtained from a control subject, wherein the control subject has HS and is not administered the composition.

In some embodiments, the level of a neutrophil serine protease (NSP) in a biological sample obtained from the subject during or subsequent to the administration period is less than: (a) the level of the NSP in a counterpart biological sample obtained from the subject prior to the administration period, and/or (b) the level of the NSP in a counterpart biological sample obtained from a control subject, wherein the control subject has hidradenitis suppurativa and is not administered the composition. In some embodiments, the level of the NSP in a biological sample obtained from the subject during or subsequent to the administration period is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, including all values and subranges that lie therebetween) less than: (a) the level of the NSP in a counterpart biological sample obtained from the subject prior to the administration period, and/or (b) the level of the NSP in a counterpart biological sample obtained from a control subject, wherein the control subject has HS and is not administered the composition.

In some embodiments, the NSP is secreted NSP, or NSP in the cytoplasmic granules. In some embodiments, the NSP is the proform of the NSP. In some embodiments, the NSP is the active form of the NSP. In some embodiments, the NSP is the secreted proform of the NSP. In some embodiments, the NSP is neutrophil elastase (NE), proteinase 3 (PR3), cathepsin G (CatG), neutrophil serine protease 4 (NSP4), or any combination thereof. In some embodiments, the NSP is cell surface-localized NSP, intracellular NSP, or a combination thereof. In some embodiments, the level of the cell surface-localized NSP or intracellular NSP is measured using flow cytometry.

In some embodiments, neutrophil elastase (NE), proteinase 3 (PR3), cathepsin G (CatG), or levels thereof may be detected and/or quantified using methods, such as, for example, western blotting, ELISA assays, enzymatic activity assays, or any combination thereof. Non-limiting examples of ELISA assays include ProteaseTag® Active NE Immunoassay, ProteaseTag® Active PR3 Immunoassay, and ProteaseTag® Active CatG Immunoassay from ProAxsis (Belfast, Northern Ireland). Non-limiting examples of activity assays include NE enzymatic kinetic assays, PR3 enzymatic kinetic assays, and CatG enzymatic kinetic assays. In some embodiments, NSP4 and its levels may be detected and/or quantified using methods, such as, for example, western blotting and enzymatic activity assays. Further details are provided in Perera et al., PNAS 109:6229-6234 (2012); Perera et al., J Immunol 191:2700-2707 (2013); Kasperkiewicz et al., PLoS One 10(7):e0132818 (2015); each of which is incorporated herein by reference in its entirety.

In some embodiments, the biological sample is sinonasal tissue, blood, serum, white blood cells (WBCs), neutrophils, skin tissue, or any combination thereof. In an embodiment of the methods disclosed herein, the biological sample is skin tissue. In some embodiments, the skin tissue comprises tissue derived from one or more skin lesions, one or more skin abscesses, one or more fistulas (e.g., one or more draining fistulas), one or more sinus tracts, one or more skin scars, or a combination thereof. In a further embodiment, the skin tissue comprises tissue derived from one or more fistulas. In a further embodiment, the one or more fistulas comprises one or more draining fistulas.

The dosage of a compound of Formula (I) will vary with the compound employed, the mode of administration, the treatment desired and the type, symptoms or severity of HS being treated. In some embodiments, the subject is administered a compound of Formula (I) at a daily dosage during the administration period of about 10 mg to about 100 mg, for example, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, including all values and subranges that lie therebetween. In some embodiments, the subject is administered a compound of Formula (I) at a daily dosage of about 10 mg to about 40 mg. In one embodiment, the subject is administered a compound of Formula (I) at a daily dosage of about 10 mg. In another embodiment, the subject is administered a compound of Formula (I) at a daily dosage of about 40 mg. In one embodiment, the compound of Formula (I) is brensocatib, or a pharmaceutically acceptable salt thereof. In another embodiment, the compound of Formula (I) is brensocatib.

In some embodiments, the daily dosage of a compound of Formula (I) during the administration period is in the range from 0.01 micrograms per kilogram body weight (g/kg) to 100 milligrams per kilogram body weight (mg/kg), for example, about 0.05 g/kg, 0.1 g/kg, 0.5 g/kg, 1 g/kg, 5 g/kg, 10 g/kg, 20 g/kg, 30 g/kg, 40 g/kg, 50 g/kg, 60 g/kg, 70 g/kg, 80 g/kg, 90 g/kg, 100 g/kg, 200 g/kg, 300 g/kg, 400 g/kg, 500 g/kg, 600 g/kg, 700 g/kg, 800 g/kg, 900 g/kg, 1 mg/kg, 20 mg/kg, 30 mg/kg, 40 mg/kg, 50 mg/kg, 60 mg/kg, 70 mg/kg, 80 mg/kg, 90 mg/kg, or 100 mg/kg, including all values and subranges that lie therebetween.

In one embodiment, the composition comprising the effective amount of a compound of Formula (I) is an oral dosage form. In a further embodiment, the compound of Formula (I) is administered as a 10 mg to 50 mg dosage form, for example, a 5 mg dosage form, a 10 mg dosage form, a 15 mg dosage form, a 20 mg dosage form, a 25 mg dosage form, a 30 mg dosage form, 35 mg dosage form, a 40 mg dosage form, a 45 mg dosage form or a 50 mg dosage form. In a further embodiment, the dosage form is a 10 mg, 25 mg or 40 mg dosage form. In a further embodiment, the dosage form is administered once daily. In one embodiment, the compound of Formula (I) is brensocatib, or a pharmaceutically acceptable salt thereof. In another embodiment, the compound of Formula (I) is brensocatib.

In some embodiments, the compound of Formula (I) is brensocatib and is present in the pharmaceutical composition in the range of about 1 mg to about 100 mg, for instance, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg about 75 mg, about 80 mg, about 85 mg, about 90 mg, about 95 mg, or about 100 mg, including all values and subranges that lie therebetween. In some embodiments, the amount of brensocatib in the pharmaceutical composition is in the range of from about 10 mg to about 40 mg. In some embodiments, the amount of brensocatib in the pharmaceutical composition is in the range of from about 25 mg to about 40 mg. In some embodiments, the amount of brensocatib in the pharmaceutical composition is in the range of from about 10 mg to about 25 mg. In some embodiments, the amount of brensocatib in the pharmaceutical composition is about 10 mg. In some embodiments, the amount of brensocatib in the pharmaceutical composition is about 40 mg. In a further embodiment, the pharmaceutical composition is administered once-a-day during the administration period. In a further embodiment, the pharmaceutical composition is administered orally during the administration period.

In the methods of treating HS provided herein, the pharmaceutical composition is administered by a suitable administration route. Administration routes include oral, enteral, transmucosal, rectal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular (including administration to skeletal, diaphragm and/or cardiac muscle), intradermal, intrapleural, intracerebral, intraarticular, intravascular or via infusion), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain). In some embodiments, the administration is by injection into the central nervous system. In one embodiment, administration is via the enteral route and is conducted through a nasogastric (NG) tube.

In a preferred embodiment, the administration route is oral. In a further embodiment, the administration is once-daily oral administration. In even a further embodiment, the pharmaceutical composition comprises 10 mg, 25 mg or 40 mg of a compound of Formula (I). In yet even a further embodiment, the compound of Formula (I) is brensocatib.

The length of the administration period in any given case may depend on the nature and severity of the condition being treated and/or prevented and be determined by the physician. In some embodiments, the administration period is about 6 months, about 12 months, about 18 months, about 24 months, or about 36 months.

In some embodiments, the administration period is about 30 days, about 35 days, about 40 days, about 45 days, about 50 days, about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months, about 30 months, about 36 months, about 4 years, about 5 years, about 10 years, about 15 years or about 20 years. In some embodiments, the compounds or compositions disclosed herein may be administered for a period of about 24 weeks. In some embodiments, the compounds or compositions disclosed herein may be administered for a period of about 52 weeks. In yet another embodiment, the administration period is at least about 30 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 24 months, at least about 30 months, at least about 36 months, at least about 4 years, at least about 5 years, at least about 10 years, at least about 15 years or at least about 20 years.

In some embodiments, the administration period for the methods provided herein is from about 6 months to about 20 years. In a further embodiment, the administration period is from about 6 months to about 18 years, from about 6 months to about 16 years, from about 6 months to about 14 years, from about 6 months to about 12 years, from about 6 months to about 10 years, from about 6 months to about 8 years, from about 6 months to about 6 years, from about 6 months to about 4 years, or from about 6 months to about 2 years.

In some embodiments, the administration period for the methods provided herein is at least about 30 days, at least about 35 days, at least about 40 days, at least about 45 days, at least about 50 days, at least about 2 months, at least about 3 months, at least about 4 months or at least about 6 months, at least about 7 months, at least about 8 months, at least about 9 months, at least about 10 months, at least about 11 months, at least about 1 year, at least about 2 years, at least about 3 years, at least about 4 years, at least about 5 years. The administration period for the methods provided herein, in another embodiment, is from about 30 days to about 180 days. In another embodiment, the administration period is from about 30 days to about 36 months, or from about 30 days to about 30 months, or from about 30 days to about 24 months, or from about 30 days to about 18 months, or from about 30 days to about 12 months, or from about 30 days to about 6 months, or from about 6 months to about 30 months, or from about 6 months to about 36 months, or from about 6 months to about 24 months, or from about 6 months to about 18 months, or from about 12 months to about 36 months, or from about 12 months to about 24 months, or from about 18 months to about 36 months.

In one embodiment, the administration period is from about 1 year to about 50 years. For example, the administration period, in one embodiment, is from about 1 year to about 40 years, from about 1 year to about 30 years, from about 1 year to about 25 years, from about 1 year to about 20 years, from about 1 year to about 15 years, from about 1 year to about 10 years, from about 1 year to about 5 years, from about 1 year to about 3 years, from about 1 year to about 2 years, from about 2 years to about 15 years, from about 2 years to about 10 years, from about 2 years to about 8 years, from about 2 years to about 5 years, from about 2 years to about 4 years, or from about 2 years to about 3 years.

In one embodiment, the subject is administered the pharmaceutical composition chronically. That is, the subject is administered the composition for their entire life, once treatment for HS is initiated.

Administration schedules, including frequency of dosage, may be determined by the physician. In some embodiments, the composition may be administered to the subject once a day or more than once a day during the administration period. In some embodiments, the composition may be administered to the subject twice a day. In some embodiments, the composition may be administered to the subject every day, every other day, every third day, every fourth day, every fifth day, or every sixth day. In some embodiments, the composition may be administered to the subject weekly, bi-weekly or every three weeks. In some embodiments, the pharmaceutical composition is administered at approximately the same time every day. In a preferred embodiment, administration of a composition comprising an effective amount of a compound of Formula (I) is conducted once daily, wherein the composition is in an oral dosage form. In a further embodiment, the compound of Formula (I) is brensocatib.

In a preferred embodiment, administration of the pharmaceutical composition is once daily during the administration period.

In another embodiment, administration is twice daily. In another embodiment, administration is once weekly, twice weekly, thrice weekly, four times weekly, five times weekly or six times weekly.

The compounds of Formula (I), or pharmaceutically acceptable salts thereof, may be used on their own, or in conjunction with the standard-of-care administered by a treating physician. Any standard-of-care therapeutic may be used in combination with the compounds disclosed herein.

In some embodiments, the compounds of Formula (I), or pharmaceutically acceptable salts thereof, are administered in combination with a secondary therapy to the subject. In some embodiments, the secondary therapy targets HS. In some embodiments, the secondary therapy is administered before, after, or concurrently with administering the pharmaceutical composition. In some embodiments, the secondary therapy comprises one or more of the following: an antibiotic, a steroid, a biologic, a hormone, a retinoid, an anti-inflammatory drug, a surgical intervention, or any combination thereof. In some embodiments, the antibiotic is applied topically. In some embodiments, the antibiotic is doxycycline, minocycline, clindamycin, rifampin, or any combination thereof. In some embodiments, the steroid is triamcinolone. In some embodiments, the biologic is an anti-TNF-alpha antibody. In some embodiments, the anti-TNF-alpha antibody is adalimumab, infliximab, or a combination thereof.

In some embodiments, the biologic is an IL-17 inhibitor, an IL-12 inhibitor, an IL-23 inhibitor, a JAK-1 inhibitor, or any combination thereof. In some embodiments, the hormone is estrogen. In some embodiments, the anti-inflammatory drug is aspirin, ibuprofen, naproxen, celecoxib, indomethacin, ketorolac, diclofenac, ketoprofen, or any combination thereof.

The term administered “in combination,” as used herein, is understood to mean that two (or more) different treatments are delivered to the subject during the course of the subject's affliction with the disorder (such as, HS), such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent” delivery. In other embodiments, the delivery of one treatment ends before the delivery of the other treatment begins, which may be referred to as “sequential” or “serial” delivery.

In some embodiments, the treatment is more effective because of combined administration. For example, the second treatment is more effective; for e.g., an equivalent effect is seen with less of the second treatment, or the second treatment reduces symptoms to a greater extent, than would be seen if the second treatment were administered in the absence of the first treatment, or the analogous situation is seen with the first treatment. The effect of the two treatments can be partially additive, wholly additive, or greater than additive (synergistic).

The compositions employed herein include an effective amount of any one or more of the compounds disclosed herein, or pharmaceutically acceptable salts thereof.

The compounds of Formula (I), or pharmaceutically acceptable salts thereof, may be used on their own, but will generally be administered in the form of a pharmaceutical composition in which the Formula (I) compound/salt (active pharmaceutical ingredient (API)) is in a composition comprising a pharmaceutically acceptable adjuvant(s), diluents(s) and/or carrier(s). In one embodiment, the pharmaceutical composition is one of the pharmaceutical compositions described in International Application Publication No. WO 2019/166626, the disclosure of which is incorporated herein by reference in its entirety for all purposes.

Conventional procedures for the selection and preparation of suitable pharmaceutical formulations are described in, for example, “Pharmaceuticals—The Science of Dosage Form Designs”, M. E. Aulton, Churchill Livingstone, 2^{nd} Ed. 2002, incorporated by reference herein in its entirety for all purposes. Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994.

Depending on the mode of administration, the pharmaceutical composition may comprise from about 0.05 to about 99 wt %, for example, from about 0.05 to about 80 wt %, or from about 0.10 to about 70 wt %, or from about 0.10 to about 50 wt %, of API, all percentages by weight being based on the total weight of the pharmaceutical composition. Unless otherwise provided herein, API weight percentages provided herein are for the respective free base form of the compound of Formula (I).

In one embodiment, the pharmaceutical composition is in the oral dosage form of a film-coated oral tablet. In another embodiment, the oral dosage form is an immediate release dosage form with rapid dissolution characteristics under in vitro test conditions. In one embodiment, the oral dosage form is administered once daily to reach the daily dosage disclosed herein. In a further embodiment, the oral dosage form is administered at approximately the same time every day, e.g., prior to breakfast. In another embodiment, the oral dosage form is administered 2× daily to reach the daily dosage disclosed herein.

In some embodiments, the compositions of the present disclosure are formulated using a pharmaceutically acceptable salt of a compound of Formula (I). Pharmaceutically-acceptable salts include, for example, acid addition salts derived from inorganic acids, e.g., hydrochloric or phosphoric acids, or from organic acids, e.g., acetic, oxalic, tartaric, mandelic, and the like. In some embodiments, the salts may be derived from inorganic bases (e.g., sodium, potassium, ammonium, calcium, or ferric hydroxides) or from organic bases (e.g., isopropylamine, trimethylamine, histidine, procaine) and the like.

The disclosure provides compositions comprising an effective amount of a compound of Formula (I), or a pharmaceutically acceptable salt thereof, for use in the methods provided herein. Any of the compounds provided herein may be used in a composition, for delivery via one of the methods provided herein.

In some embodiments, the composition is in a solid form, such as a lyophilized powder suitable for reconstitution, a liquid solution, suspension, emulsion, tablet, pill, capsule, sustained release formulation, or powder. In some embodiments, delivery vehicles such as liposomes, nanocapsules, microparticles, microspheres, lipid particles, vesicles, and the like, may be used.

In some embodiments, the compositions disclosed herein further comprise at least one pharmaceutically acceptable carrier, excipient, and/or vehicle, for example, solvents, buffers, solutions, dispersion media, coatings, antibacterial and antifungal agents, isotonic and absorption delaying agents. In some embodiments, the pharmaceutically acceptable carrier, excipient, and/or vehicle may comprise saline, buffered saline, dextrose, water, glycerol, sterile isotonic aqueous buffer, and combinations thereof. In some embodiments, the pharmaceutically acceptable carrier, excipient, and/or vehicle comprises phosphate buffered saline, sterile saline, lactose, sucrose, calcium phosphate, dextran, agar, pectin, peanut oil, sesame oil, pharmaceutical grades of mannitol, lactose, starch, magnesium stearate, sodium saccharine, cellulose, magnesium carbonate, polyol (e.g., glycerol, propylene glycol, and liquid polyethylene glycol, and the like) or suitable mixtures thereof. In some embodiments, the compositions disclosed herein further comprise minor amounts of emulsifying or wetting agents, or pH buffering agents.

In some embodiments, the compositions disclosed herein further comprise other conventional pharmaceutical ingredients, such as preservatives, or chemical stabilizers, such as chlorobutanol, potassium sorbate, sorbic acid, sulfur dioxide, propyl gallate, the parabens, ethyl vanillin, glycerin, phenol, parachlorophenol or albumin. In some embodiments, the compositions disclosed herein may further comprise antibacterial and antifungal agents, such as, parabens, chlorobutanol, phenol, sorbic acid or thimerosal; isotonic agents, such as, sugars or sodium chloride and/or agents delaying absorption, such as, aluminum monostearate and gelatin.

In some embodiments, the amount of compounds of Formula (I) present in a pharmaceutical composition depends on the mode of administration. In some embodiments, the pharmaceutical composition comprises from about 0.05% w to about 99% w (percent by weight), for example, about 0.5% w, about 1% w, about 5% w, about 10% w, about 15% w, about 20% w, about 25% w, about 30% w, about 35% w, about 40% w, about 45% w, about 50% w, about 55% w, about 60% w, about 65% w, about 70% w, about 75% w, about 80% w, about 85% w, about 90% w, about 95% w, about 98% w, or about 99% w, including all values and subranges that lie therebetween. In some embodiments, the pharmaceutical composition comprises from about 0.05% w to about 80% w, or from about 0.10% w to about 70% w, or from about 0.10% w to about 50% w, of active ingredient (compound of Formula (I)), all percentages by weight being based on total composition.

In some embodiments, the adjuvant(s), diluent(s) or carrier(s) present in the pharmaceutical composition are selected based on the mode of administration. For instance, for oral administration the compound of the disclosure may be admixed with adjuvant(s), diluent(s) or carrier(s), for example, lactose, saccharose, sorbitol, mannitol; starch, for example, potato starch, corn starch or amylopectin; cellulose derivative; binder, for example, gelatine or polyvinylpyrrolidone; disintegrant, for example cellulose derivative, and/or lubricant, for example, magnesium stearate, calcium stearate, polyethylene glycol, wax, paraffin, and the like, and then compressed into tablets. If coated tablets are required, the cores, prepared as described above, may be coated with a suitable polymer dissolved or dispersed in water or readily volatile organic solvent(s). Alternatively, the tablet may be coated with a concentrated sugar solution which may contain, for example, gum arabic, gelatine, talcum and titanium dioxide.

For the preparation of soft gelatine capsules, the compound of the disclosure may be admixed with, for example, a vegetable oil or polyethylene glycol. Hard gelatine capsules may contain granules of the compound using pharmaceutical excipients like the above-mentioned excipients for tablets. Also liquid or semisolid formulations of the compound of the disclosure may be filled into hard gelatine capsules.

In some embodiments, the form of the pharmaceutical composition depends on the mode of administration. For instance, in one oral administration embodiment, the oral dosage form is a film-coated oral tablet. In a further embodiment, the dosage form is an immediate release dosage form with rapid dissolution characteristics under in vitro test conditions.

In one embodiment, the composition is an oral disintegrating tablet (ODT). ODTs differ from traditional tablets in that they are designed to be dissolved on the tongue rather than swallowed whole.

In one embodiment, the composition is an oral thin film or an oral disintegrating film (ODF). Such formulations, when placed on the tongue, hydrate via interaction with saliva, and releases the active compound from the dosage form. The ODF, in one embodiment, contains a film-forming polymer such as hydroxypropylmethylcellulose (HPMC), hydroxypropyl cellulose (HPC), pullulan, carboxymethyl cellulose (CMC), pectin, starch, polyvinyl acetate (PVA) or sodium alginate.

Liquid preparations for oral administration may be in the form of syrups, solutions or suspensions. Solutions, for example, may contain the compound of the disclosure, the balance being sugar and a mixture of ethanol, water, glycerol and propylene glycol. Optionally such liquid preparations may contain coloring agents, flavoring agents, saccharine and/or carboxymethylcellulose as a thickening agent. Furthermore, other excipients known to those skilled in art may be used when making formulations for oral use.

The compounds of the disclosure may be prepared, in known manner, in a variety of ways. For example, in one embodiment, compounds of Formula (I) are prepared according to the methods set forth in U.S. Pat. No. 9,522,894, incorporated by reference herein in its entirety for all purposes. A composition used in the methods provided herein, in one embodiment, is prepared by one of the methods disclosed in International Application Publication No. WO 2019/166626, the disclosure of which is incorporated herein by reference in its entirety for all purposes.

As provided throughout, according to the methods provided herein, a compound of Formula (I) can be administered as a pharmaceutically acceptable salt. A pharmaceutically acceptable salt of a compound of Formula (I) may be advantageous due to one or more of its chemical or physical properties, such as stability in differing temperatures and humidities, or a desirable solubility in H₂O, oil, or other solvent. In some instances, a salt may be used to aid in the isolation or purification of the compound of Formula (I).

Where the compound of Formula (I) is sufficiently acidic, pharmaceutically acceptable salts include, but are not limited to, an alkali metal salt, e.g., Na or K, an alkali earth metal salt, e.g., Ca or Mg, or an organic amine salt. Where the compound of Formula (I) is sufficiently basic, pharmaceutically acceptable salts include, but are not limited to, inorganic or organic acid addition salts.

There may be more than one cation or anion depending on the number of charged functions and the valency of the cations or anions. For reviews on suitable salts, and pharmaceutically acceptable salts amenable for use herein, see Berge et al. (1977). J. Pharm. Sci., 66, 1-19 or “Handbook of Pharmaceutical Salts: Properties, selection and use”, P. H. Stahl, P. G. Vermuth, IUPAC, Wiley-VCH, 2002, incorporated by reference herein in its entirety for all purposes. The compounds of Formula (I) may form mixtures of its salt and co-crystal forms. It is also to be understood that the methods provided herein can employ such salt/co-crystal mixtures of the compound of Formula (I).

Salts and co-crystals may be characterized using well known techniques, for example X-ray powder diffraction, single crystal X-ray diffraction (for example to evaluate proton position, bond lengths or bond angles), solid state NMR, (to evaluate for example, C, N or P chemical shifts) or spectroscopic techniques (to measure for example, O—H, N—H or COOH signals and IR peak shifts resulting from hydrogen bonding).

In another embodiment of the methods, the pharmaceutical composition administered to the patient is Composition (A) comprising:
(a) from about 1 to about 30 wt % of the compound of Formula (I), or a pharmaceutically acceptable salt thereof,
(b) from about 45 to about 85 wt % of a pharmaceutical diluent;
(c) from about 6 to about 30 wt % of a compression aid;
(d) from about 1 to about 15 wt % of a pharmaceutical disintegrant;
(e) from about 0.00 to about 2 wt % of a pharmaceutical glidant; and
(f) from about 1 to about 10 wt % of a pharmaceutical lubricant;
wherein the components add up to 100 wt %.

In a further embodiment, the compound of Formula (I) is brensocatib. In one embodiment, brensocatib is in polymorphic Form A as disclosed in U.S. Pat. No. 9,522,894. In another embodiment, brensocatib is characterized by one of the X-ray powder diffraction patterns described in International Application Publication No. WO 2019/166626.

In some embodiments of the methods, Composition (A) comprises the compound of Formula (I), e.g., brensocatib, in an amount from about 1 to about 25 wt %; from about 1 to about 20 wt %; from about 1 to about 15 wt %; from about 1 to about 10 wt %; from about 1 to about 5 wt %, or from about 1 to about 3 wt % of the total weight of the composition.

In some embodiments of the methods, Composition (A) comprises the compound of Formula (I), e.g., brensocatib, in an amount from about 1.5 to about 30 wt %; from about 1.5 to about 25 wt %; from about 1.5 to about 20 wt %; from about 1.5 to about 15 wt %; from about 1.5 to about 10 wt %; or from about 1.5 to about 5 wt % of the total weight of the composition.

In some embodiments of the methods, Composition (A) comprises the compound of Formula (I), e.g., brensocatib, in an amount from about 3 to about 30 wt %; from about 3 to about 25 wt %; from about 3 to about 20 wt %; from about 3 to about 15 wt %; from about 3 to about 10 wt %; or from about 3 to about 5 wt % of the total weight of the composition. In a further embodiment, the compound of Formula (I) is present at from about 3 to about 10 wt % of the total weight of the composition. In a further embodiment, the compound of Formula (I) is brensocatib, or a pharmaceutically acceptable salt thereof.

In some embodiments of the methods, Composition (A) comprises the compound of Formula (I), e.g., brensocatib, in an amount of about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt % or about 30 wt % of the total weight of the composition.

In some embodiments of the methods, Composition (A) comprises one or more pharmaceutical diluents selected from the group consisting of microcrystalline cellulose, calcium carbonate, calcium phosphate, calcium sulfate, cellulose acetate, erythritol, ethylcellulose, fructose, inulin, isomalt, lactitol, lactose, magnesium carbonate, magnesium oxide, maltitol, maltodextrin, maltose, mannitol, polydextrose, polyethylene glycol, pullulan, simethicone, sodium bicarbonate, sodium carbonate, sodium chloride, sorbitol, starch, sucrose, trehalose, xylitol, and a combination of the foregoing. In one embodiment, Composition (A) comprises two or more pharmaceutical diluents. In another embodiment, Composition (A) comprises one pharmaceutical diluent. In a further embodiment, the pharmaceutical diluent is microcrystalline cellulose. Microcrystalline cellulose is a binder/diluent in oral tablet and capsule formulations and can be used in dry-granulation, wet-granulation, and direct-compression processes.

In some embodiments of the methods, Composition (A) comprises one or more pharmaceutical diluents in an amount from about 45 to about 80 wt %, from about 45 to about 75 wt %, from about 45 to about 70 wt %, from about 45 to about 65 wt %, from about 45 to about 60 wt %, or from about 45 to about 55 wt % of the total weight of the composition. In a further embodiment, the one or more pharmaceutical diluents comprises microcrystalline cellulose. In even a further embodiment, the compound of Formula (I) is brensocatib, or a pharmaceutically acceptable salt thereof.

In some embodiments of the methods, Composition (A) comprises one or more pharmaceutical diluents in an amount from about 50 to about 85 wt %, from about 50 to about 75 wt %, from about 55 to about 85 wt %, from about 55 to about 70 wt %, from about 60 to about 85 wt %, from about 65 to about 85 wt %, from about 70 to about 85 wt %, or from about 75 to about 85 wt % of the total weight of the composition. In a further embodiment, the one or more pharmaceutical diluents is present at from about 55 to about 70 wt % of the total weight of the composition. In a further embodiment, the one or more pharmaceutical diluents comprises microcrystalline cellulose. In even a further embodiment, the compound of Formula (I) is brensocatib, or a pharmaceutically acceptable salt thereof.

In some embodiments of the methods, Composition (A) comprises one or more pharmaceutical diluents in an amount of about 45 wt %, about 50 wt %, about 55 wt %, about 60 wt %, about 65 wt %, about 70 wt %, about 75 wt %, about 80 wt % or about 85 wt % of the total weight of the composition.

In some embodiments of the methods, the one or more pharmaceutical diluents in Composition (A) is microcrystalline cellulose. In other embodiments, the one or more pharmaceutical diluents comprises calcium carbonate, calcium phosphate, calcium sulfate, cellulose acetate, erythritol, ethylcellulose, fructose, inulin, isomalt, lactitol, magnesium carbonate, magnesium oxide, maltitol, maltodextrin, maltose, mannitol, polydextrose, polyethylene glycol, pullulan, simethicone, sodium bicarbonate, sodium carbonate, sodium chloride, sorbitol, starch, sucrose, trehalose and xylitol.

In the present disclosure, the terms “disintegrant” and “disintegrants” are intended to be interpreted in the context of pharmaceutical formulation science. Accordingly, a disintegrant in the Composition (A) may be, for example: alginic acid, calcium alginate, carboxymethylcellulose calcium, chitosan, croscarmellose sodium, crospovidone, glycine, guar gum, hydroxypropyl cellulose, low-substituted hydroxypropyl cellulose, magnesium aluminum silicate, methylcellulose, povidone, sodium alginate, sodium carboxymethylcellulose, sodium starch glycolate, starch, or a combination thereof.

In some embodiments of the methods, the one or more disintegrants in Composition (A) is sodium starch glycolate. In one embodiment, the amount of the disintegrants present in Composition (A) is between 2% and 8% of the total weight of the composition. In a further embodiment, the amount of the disintegrants is about 2 wt %, about 2.5 wt %, about 3 wt %, about 3.5 wt %, about 4 wt % or about 4.5 wt % of the total weight of the composition. The physical properties of sodium starch glycolate, and hence its effectiveness as a disintegrant, are affected by the degree of crosslinkage, extent of carboxymethylation, and purity.

In some embodiments of the methods, the one or more pharmaceutical disintegrants in Composition (A) comprises croscarmellose sodium.

In some embodiments of the methods, Composition (A) comprises one or more pharmaceutical disintegrants in an amount from about 2 to about 14 wt %, from about 2 to about 13 wt %, from about 2 to about 12 wt %, from about 2 to about 11 wt %, from about 2 to about 10 wt %, from about 2 to about 9 wt %, from about 2 to about 8 wt %, from about 2 to about 7 wt %, from about 2 to about 6 wt %, from about 2 to about 5 wt %, from about 3.5 to about 4.5 wt % of the total weight of the composition. In a further embodiment, the one or more pharmaceutical disintegrants is present at from about 3.5 to about 4.5 wt % of the total weight of the pharmaceutical composition. In a further embodiment, the one or more pharmaceutical disintegrants is sodium starch glycolate. In a further embodiment, the one or more pharmaceutical diluents comprises microcrystalline cellulose. In even a further embodiment, the compound of Formula (I) is brensocatib, or a pharmaceutically acceptable salt thereof.

In the present disclosure, the terms “glidants” and “gliding agents” are intended to be interpreted in the context of pharmaceutical formulation science. Accordingly, a glidant in Composition (A) may be, for example: silicon dioxide, colloidal silicon dioxide, powdered cellulose, hydrophobic colloidal silica, magnesium oxide, magnesium silicate, magnesium trisilicate, sodium stearate and talc.

Accordingly, in some embodiments of the methods, the one or more pharmaceutical glidants in Composition (A) is selected from silicon dioxide, colloidal silicon dioxide, powdered cellulose, hydrophobic colloidal silica, magnesium oxide, magnesium silicate, magnesium trisilicate, sodium stearate, talc, or a combination of the foregoing. In one embodiment, the glidant is silicon dioxide. Its small particle size and large specific surface area give it desirable flow characteristics that are exploited to improve the flow properties of dry powders in a number of processes such as tableting and capsule filling. Typical silicon dioxide concentrations for use herein range from about 0.05 to about 1.0 wt %. Porous silica gel particles may also be used as a glidant, which may be an advantage for some formulations, with typical concentrations of 0.25-1%.

In some embodiments of the methods, Composition (A) comprises one or more pharmaceutical glidants in an amount from about 0.00 to about 1.75 wt %; from about 0.00 to about 1.50 wt %; from about 0.00 to about 1.25 wt %; from about 0.00 to about 1.00 wt %; from about 0.00 to about 0.75 wt %; from about 0.00 to about 0.50 wt %; from about 0.00 to about 0.25 wt %; from about 0.00 to about 0.20 wt % of the total weight of the composition. In a further embodiment, the one or more pharmaceutical glidants comprises silicon dioxide. In a further embodiment, the one or more pharmaceutical disintegrants is sodium starch glycolate. In a further embodiment, the one or more pharmaceutical diluents comprises microcrystalline cellulose. In even a further embodiment, the compound of Formula (I) in Composition (A) is brensocatib, or a pharmaceutically acceptable salt thereof.

In some embodiments of the methods, Composition (A) comprises one or more pharmaceutical glidants in an amount from about 0.05 to about 2 wt %; from about 0.05 to about 1.75 wt %; from about 0.05 to about 1.50 wt %; from about 0.05 to about 1.25 wt %; from about 0.05 to about 1.00 wt %; from about 0.05 to about 0.75 wt %; from about 0.05 to about 0.50 wt %; from about 0.05 to about 0.25 wt %; or from about 0.05 to about 0.20 wt % of the total weight of the composition. In a further embodiment, the one or more pharmaceutical glidants is present at from about 0.05 to about 0.25 wt % of the total weight of the composition. In a further embodiment, the one or more pharmaceutical glidants comprises silicon dioxide. In a further embodiment, the one or more pharmaceutical disintegrants is sodium starch glycolate. In a further embodiment, the one or more pharmaceutical diluents comprises microcrystalline cellulose. In even a further embodiment, the compound of Formula (I) in Composition (A) is brensocatib, or a pharmaceutically acceptable salt thereof.

In some embodiments of the methods, Composition (A) comprises one or more pharmaceutical glidants in an amount from about 0.05 to about 2 wt %; from about 0.10 to about 2 wt %; from about 0.2 to about 2 wt %; from about 0.3 to about 2 wt %; or from about 0.40 to about 2 wt % of the total weight of the composition. In a further embodiment, the one or more pharmaceutical glidants comprises silicon dioxide. In a further embodiment, the one or more pharmaceutical disintegrants is sodium starch glycolate. In a further embodiment, the one or more pharmaceutical diluents comprises microcrystalline cellulose. In even a further embodiment, the compound of Formula (I) in Composition (A) is brensocatib, or a pharmaceutically acceptable salt thereof.

In the present disclosure, the terms “lubricant” and “lubricants”, as used herein, are intended to be interpreted in the context of pharmaceutical formulation science. Accordingly, a lubricant may be, for example calcium stearate, glyceryl behenate, glyceryl monostearate, glyceryl palmitostearate, a mixture of behenate esters of glycerine (e.g., a mixture of glyceryl bihenehate, tribehenin and glyceryl behenate), leucine, magnesium stearate, myristic acid, palmitic acid, poloxamer, polyethylene glycol, potassium benzoate, sodium benzoate, sodium lauryl sulfate, sodium stearate, sodium stearyl fumarate, stearic acid, talc, tribehenin and zinc stearate.

Accordingly, in some embodiments of the methods, the one or more pharmaceutical lubricants in Composition (A) are selected from the group consisting of calcium stearate, glyceryl behenate, glyceryl monostearate, glyceryl palmitostearate, a mixture of behenate esters of glycerine (e.g., a mixture of glyceryl bihenehate, tribehenin and glyceryl behenate), leucine, magnesium stearate, myristic acid, palmitic acid, poloxamer, polyethylene glycol, potassium benzoate, sodium benzoate, sodium lauryl sulfate, sodium stearate, sodium stearyl fumarate, stearic acid, talc, tribehenin and zinc stearate. In other embodiments, the one or more pharmaceutical lubricants are selected from the group consisting of calcium stearate, glyceryl behenate, glyceryl monostearate, glyceryl palmitostearate, a mixture of behenate esters of glycerine (e.g., a mixture of glyceryl bihenehate, tribehenin and glyceryl behenate), leucine, magnesium stearate, myristic acid, palmitic acid, poloxamer, polyethylene glycol, potassium benzoate, sodium benzoate, sodium lauryl sulfate, sodium stearate, stearic acid, talc, tribehenin and zinc stearate.

In some embodiments of the methods, Composition (A) comprises one or more pharmaceutical lubricants and the lubricant is not sodium stearyl fumarate. In a further embodiment, the compound of Formula (I) in Composition (A) is brensocatib, or a pharmaceutically acceptable salt thereof.

In one embodiment of the methods, Composition (A) includes glycerol behenate as the lubricant.

In some embodiments of the methods, the one or more pharmaceutical lubricants in Composition (A) comprises glyceryl behenate, magnesium stearate, stearic acid, or a combination thereof.

In one embodiment of the methods, the lubricant in Composition (A) is glyceryl behenate, magnesium stearate, or a combination thereof.

In one embodiment of the methods, the one or more pharmaceutical lubricants in Composition (A) comprises sodium stearyl fumarate and/or one or more behenate esters of glycerine.

In some embodiments of the methods, Composition (A) comprises one or more pharmaceutical lubricants in an amount from about 1 wt % to about 9 wt %, from about 1 wt % to about 8 wt %, from about 1 wt % to about 7 wt %, from about 1 wt % to about 6 wt %, from about 1 wt % to about 5 wt %, from about 2 wt % to about 10 wt %, from about 2.5 wt % to about 10 wt %, from about 2 wt % to about 8 wt %, from about 2 wt % to about 7 wt %, from about 2 wt % to about 6 wt %, from about 2 wt % to about 5 wt %, from about 2 wt % to about 4.5 wt %, or from about 2.5 wt % to about 4.5 wt % of the total weight of the composition. In a further embodiment, the one or more pharmaceutical lubricants is present at from about 2.5 to about 4.5 wt % of the total weight of the composition. In a further embodiment, the one or more pharmaceutical lubricants in Composition (A) is glycerol behenate. In a further embodiment, the one or more pharmaceutical glidants in Composition (A) comprises silicon dioxide. In a further embodiment, the one or more pharmaceutical disintegrants in Composition (A) is sodium starch glycolate. In a further embodiment, the one or more pharmaceutical diluents in Composition (A) comprises microcrystalline cellulose. In even a further embodiment, the compound of Formula (I) in Composition (A) is brensocatib, or a pharmaceutically acceptable salt thereof.

In one embodiment of the methods, the one or more pharmaceutical lubricants in Composition (A) consists of sodium stearyl fumarate and/or one or more behenate esters of glycerine or a mixture thereof.

In another embodiment of the methods, the one or more pharmaceutical lubricants in Composition (A) consists of sodium stearyl fumarate, glyceryl dibehenate, glyceryl behenate, tribehenin or any mixture thereof.

In one embodiment of the methods, the one or more pharmaceutical lubricants in Composition (A) comprises sodium stearyl fumarate. In another embodiment, the one or more pharmaceutical lubricants in Composition (A) consists of sodium stearyl fumarate.

In one embodiment of the methods, the one or more pharmaceutical lubricants in Composition (A) comprises one or more behenate esters of glycerine (i.e., one or more of glyceryl dibehenate, tribehenin and glyceryl behenate).

In one embodiment of the methods, the compression aid in Composition (A) is dicalcium phosphate dihydrate (also known as dibasic calcium phosphate dihydrate) (DCPD). DCPD is used in tablet formulations both as an excipient and as a source of calcium and phosphorus in nutritional supplements.

In one embodiment of the methods, Composition (A) comprises the compression aid, e.g., DCPD, in an amount from about 10 to about 30 wt %, including about 16 wt %, about 17 wt %, about 18 wt %, about 19 wt %, about 20 wt %, about 21 wt %, about 22 wt %, about 23 wt %, or about 24 wt % of the total weight of the composition. In a further embodiment, the compression aid is present at about 20 wt % of the total weight of the composition.

In one embodiment of the methods, Composition (A) comprises the compression aid, e.g., DCPD, in an amount from about 10 to about 25 wt %, from about 10 to about 20 wt %, from about 10 to about 15 wt %, from about 15 to about 25 wt %, or from about 20 to about 25 wt %, or from about 18 to about 22 wt % of the total weight of the composition. In a further embodiment, the compression aid is present at from about 18 to about 22 wt % of the total weight of the composition. In a further embodiment, the compression aid is DCPD. In a further embodiment, the one or more pharmaceutical lubricants in Composition (A) is glycerol behenate. In a further embodiment, the one or more pharmaceutical glidants in Composition (A) comprises silicon dioxide. In a further embodiment, the one or more pharmaceutical disintegrants in Composition (A) is sodium starch glycolate. In a further embodiment, the one or more pharmaceutical diluents in Composition (A) comprises microcrystalline cellulose. In even a further embodiment, the compound of Formula (I) in the exemplary composition is brensocatib, or a pharmaceutically acceptable salt thereof.

In one embodiment of the methods, the pharmaceutical composition administered to the patient is Composition (B) comprising:
(a) from about 1 to about 30 wt % of the compound of Formula (I), or a pharmaceutically acceptable salt thereof,
(b) from about 55 to about 75 wt % of a pharmaceutical diluent;
(c) from about 15 to about 25 wt % of a compression aid;
(d) from about 3 to about 5 wt % of a pharmaceutical disintegrant;
(e) from about 0.00 to about 1 wt % of a pharmaceutical glidant; and
(f) from about 2 to about 6 wt % of a pharmaceutical lubricant;
wherein the components add up to 100 wt %.

In some embodiments of the methods where Composition (B) is administered to the patient, the identity of the pharmaceutical diluent, compression aid, pharmaceutical disintegrant, pharmaceutical glidant, and pharmaceutical lubricant in the composition may be one of those described above for Composition (A). In other embodiments, the amount of the pharmaceutical diluent, compression aid, pharmaceutical disintegrant, pharmaceutical glidant, and pharmaceutical lubricant in Composition (B) may also be one of those described above for Composition (A), as long as the amount is within the corresponding broader range recited above for Composition (B).

The pharmaceutical compositions disclosed herein, including Compositions (A) and (B), may be in a solid dosage form suitable for oral administration to a human being. For example, the pharmaceutical composition is a pharmaceutical tablet. Pharmaceutical tablets may be prepared using methods known to those skilled in the art including, for example, dry mixing/direct compression process as described in International Application Publication No. WO 2019/166626. In some embodiments, the pharmaceutical tablet comprises a tablet core wherein the tablet core comprises the pharmaceutical composition as disclosed herein and wherein the tablet core has a coating. In some embodiments, the coating is a film coating. The film coating may be applied using conventional methods known to those skilled in the art. A functional coating can be used to provide protection against, for example, moisture ingress or degradation by light. Additionally, a functional coating may be used to modify or control the release of the compound of Formula (I), e.g., brensocatib, from the composition. The coating may comprise, for example, about 0.2 to about 10 wt % of the total weight of the pharmaceutical composition, e.g., from about 0.2 to about 4 wt %, from about 0.2 to about 3 wt %, from about 1 to about 6 wt %, or from about 2 to about 5 wt % of the total weight of the pharmaceutical composition.

### EXAMPLES

The present disclosure is further illustrated by reference to the following Examples. However, it should be noted that the Examples, like the embodiments described above, are illustrative and are not to be construed as restricting the scope of the invention in any way.

### Example 1—a Phase 2b, Randomized, Double-Blind, Placebo-Controlled, Multicenter Study of the Efficacy and Safety of Brensocatib in Adults with Moderate to Severe Hidradenitis Suppurativa

Brensocatib, a compound of Formula (I), is an oral reversible DPP1 inhibitor. DPP1 activates neutrophil serine proteases (NSPs) which, when released from neutrophils, are proinflammatory and generally destructive to the surrounding tissues. As neutrophils are the predominant leukocyte infiltrate in HS lesions, and neutrophil counts, as well as neutrophil extracellular traps (NETs), were significantly associated with HS disease severity, inhibition of DPP1 activity by brensocatib in patients with moderate to severe HS is expected to lead to a reduction in the formation of active NSPs, thereby preventing the propagation of the inflammatory response, and possibly the presence of NETs, which are implicated in local and systemic immune dysregulation. As a result, brensocatib treatment is expected to result in a clinically meaningful reduction in the number and severity of HS lesions (abscesses, tunnels (both draining and nondraining), and nodules (both inflammatory and noninflammatory).

This example describes a Phase 2b, randomized, double-blind, placebo-controlled, study to assess the efficacy and safety of brensocatib in adults with moderate to severe hidradenitis suppurativa. Brensocatib is the International Nonproprietary Name for (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide

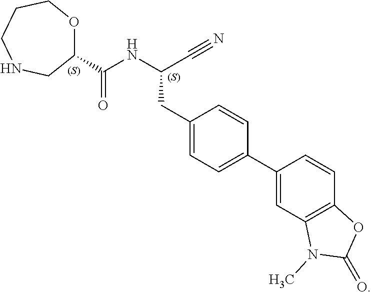

A total of approximately 204 eligible participants (≥18 to ≤75 years of age) are randomized into three treatment arms in a 1:1:1 ratio, with approximately 68 participants per treatment arm, to receive once daily oral dosing of 10 mg brensocatib, 40 mg brensocatib, and matching placebo, respectively, for 16 weeks (Period 1), followed by an open-label extension period of 36 weeks (Period 2), during which all the participants receive once daily oral dosing of 40 mg brensocatib. The treatment regimen permits background therapy for HS (e.g., topical over-the-counter antiseptic/antibacterial treatments, prestudy analgesics). Efficacy parameters include Hidradenitis Suppurativa Clinical Response (HiSCR), HiSCR75, Hidradenitis Suppurativa-Investigator's Global Assessment (HS-IGA), total AN count, draining tunnel count, HS flares, Hidradenitis Suppurativa Severity Score 4 (IHS4), Dermatology Life Quality Index (DLQI), and Numeric Rating Scale (NRS) for skin pain. Safety parameters include adverse events (AEs), clinical laboratory test results, vital sign measurements, 12-lead ECG measurements, and physical examination results. Adverse events of special interest (AESIs) include hyperkeratosis, periodontitis/gingivitis, and infection. Pharmacokinetic (PK) parametes include plasma concentrations of brensocatib.

Brensocatib oral tablets are used in the study. The tablets are round, biconvex, and brown film-coated and are considered as an immediate-release dosage form. The tablets contain the equivalent of 10 mg or 40 mg of brensocatib drug substance and are identical in size and appearance. Each film-coated tablet contains active ingredient of brensocatib drug substance and compendial ingredients: microcrystalline cellulose, dibasic calcium phosphate dihydrate, sodium starch glycolate, silicon dioxide, and glyceryl behenate. The matching placebo without the active ingredient is a film-coated tablet identical to brensocatib film-coated tablets in shape, size, and color.

### Subject Eligibility Criteria

TABLE 1
Key inclusion criteria for the study

1. Adult male or female participants ≥18 to ≤75 years of age
2. Diagnosis of HS for at least 6 months before the Screening Visit
3. Moderate or severe HS defined as a total of ≥6 inflammatory lesions
for at least 8 weeks before the Baseline Visit
4. HS lesions in at least 2 distinct anatomic areas, 1 of which must
be at least IHS4 score ≥4 at both the Screening and Baseline Visits

### Study Design

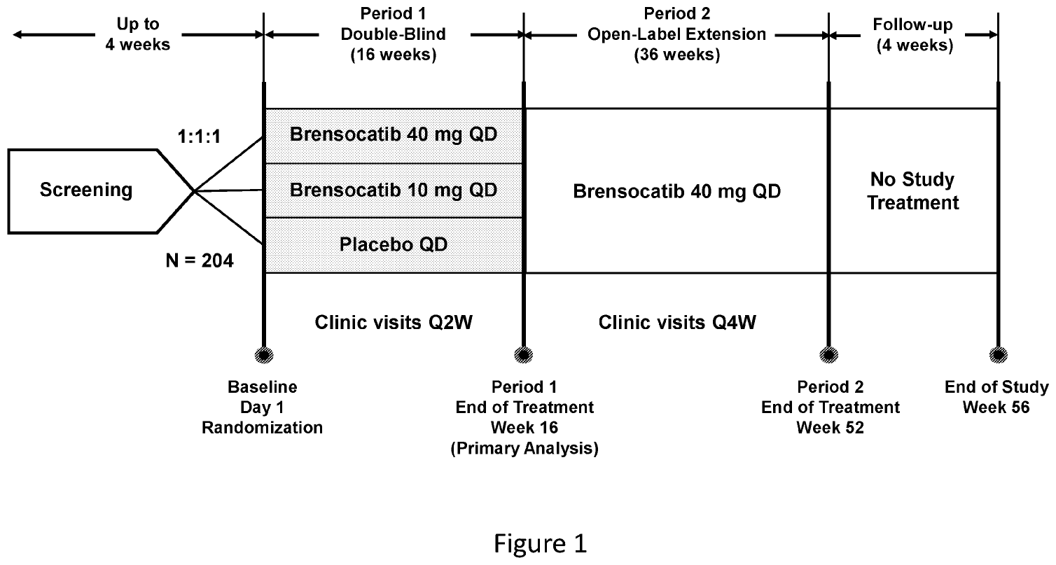

FIG. 1 provides a schematic diagram of the study design. In this Phase 2b, randomized, double-blind, parallel-group, placebo-controlled, dose-ranging, multicenter study, brensocatib is administered once daily (QD) for up to 52 weeks in approximately 204 adult male and female participants (≥18 to ≤75 years of age) with moderate to severe HS. The study includes 4 study periods, with efficacy and safety assessments, as well as blood sample collections for PK/PD analysis, performed throughout the study.

Screening Period—Participant eligibility is determined during a Screening Period of at least 7 and up to 28 days (4 weeks).

Treatment Period 1—Eligible participants are randomized using an Interactive Web Response System (IWRS) to receive double-blind brensocatib 10 mg, brensocatib 40 mg, or placebo film-coated tablet QD by mouth for 16 weeks in a 1:1:1 ratio, with approximately 68 participants per treatment arm. Randomization is stratified based on total IHS4 score (4-10 moderate vs >11 severe), previous anti-tumor necrosis factor alpha (TNFα) treatment (Yes vs No), and participation in the pharmacodynamic (PD) tissue substudy (Yes vs No). The PD tissue substudy includes up to 30 evaluable participants (up to 10 per treatment arm) at select study sites, in which tissue samples are collected from active HS lesions at designated study visits in Period 1 and Period 2. An evaluable participant is considered one who receives at least 1 dose of study treatment and has at least 1 interpretable predose and 1 postdose tissue sample. Investigators (including clinicians providing care to the participant), Sponsor, and participants/care providers are blinded to Period 1 study treatment.

Treatment Period 2—Participants who complete study treatment in Period 1 directly roll over into Period 2 during which all participants receive open-label brensocatib 40 mg film-coated tablet QD by mouth for 36 weeks.

Follow-up Period—Participants are followed for 4 weeks after the last dose of study treatment.

Throughout the study, participants are allowed to use topical over-the-counter antiseptic/antibacterial treatments (including HS wound care dressings), the same stable regimen of prestudy analgesics for HS (i.e., ibuprofen, acetaminophen/paracetamol, and tramadol; opioid analgesics are prohibited, except for tramadol), and the same stable regimen of prestudy oral antibiotics for HS (i.e., doxycycline and minocycline).

The total duration of study treatment is approximately 52 weeks:
Period 1 (double-blind treatment): 16 weeks
Period 2 (open-label extension): 36 weeks

Participants randomized to brensocatib in Period 1 are exposed to brensocatib for approximately 52 weeks (16 weeks in Period 1 plus 36 weeks in Period 2). Participants randomized to placebo in Period 1 are exposed to brensocatib in Period 2 for approximately 36 weeks.

Study treatments are presented in Table 2 below. In both study periods, brensocatib or placebo film-coated tablets are administered QD by mouth, with water and with or without food, at approximately the same time each morning. On study visit days, study treatment is administered at the study site by study personnel. At the Baseline Visit (Day 1), study treatment is administered after an overnight fast of at least 8 hours.

TABLE 2
Study treatments

| | | | |
|---|---|---|---|
| Treatment | Unit Dose | Dosage | Route and Frequency |
| Name | Strengths | Formulation | of Administration |

Period 1 (double-blind)

| | | | |
|---|---|---|---|
| Brensocatib | 10 mg | Tablet | PO, QD |
| Brensocatib | 40 mg | Tablet | PO, QD |
| Placebo | Not | Tablet | PO, QD |
| | Applicable | | |

Period 2 (open-label)

| | | | |
|---|---|---|---|
| Brensocatib | 40 mg | Tablet | PO, QD |

PO = orally; QD = once daily

### Outcomes Assessment

The objectives and variables (endpoints) of the study are shown in Table 3.

TABLE 3
Objectives and Endpoints of the Study

| | | |
|---|---|---|
| | Variable | Population Level |
| Objective | (or Endpoint) | Summary |

Primary

| | | |
|---|---|---|
| To evaluate the effect of | Responder status for achieving HiSCR at Week | Odds ratio |
| brensocatib compared | 16^{a} | |
| with placebo | | |

Secondary
Period 1 (Double-Blind)

| | | |
|---|---|---|
| To evaluate the effect of | Responder status for achieving HiSCR75 at Week | Odds ratio |
| brensocatib compared | 16^{b} | |
| with placebo | Responder status for achieving ≥2-point decrease | Odds ratio |
| | from Baseline in HS-IGA at Week 16 | |
| | Change from Baseline in total AN count at Week | Mean difference |
| | 16 | |
| | Change from Baseline in draining tunnel count at | Mean difference |
| | Week 16 | |
| | Responder status for remaining free from HS | Odds ratio |
| | flares by Week 16^{c} | |
| | Change from Baseline in IHS4 at Week 16 | Mean difference |
| | Change from Baseline on DLQI at Week 16 | Mean difference |
| | Change from Baseline in mean daily NRS for skin | Mean difference |
| | pain over Week 16 | |
| To evaluate the safety | Adverse events over 16 weeks of treatment | Incidence (%) |
| and tolerability of | Clinical laboratory test results, vital signs, and | Mean absolute |
| brensocatib compared | electrocardiograms (ECGs) over 16 weeks of | value and mean |
| with placebo | treatment | change from |
| | | Baseline |
| To evaluate the systemic | Brensocatib plasma concentrations over 16 weeks | Descriptive |
| exposure of brensocatib | of treatment | statistics |

Period 2 (Open-Label)

| | | |
|---|---|---|
| To assess the long-term | Responder status for achieving HiSCR at Week 52 | Percentage of |
| effect of brensocatib | | responders |
| | Responder status for achieving HiSCR75 at Week | Percentage of |
| | 52 | responders |
| | Responder status for achieving ≥2-point decrease | Percentage of |
| | from Baseline in HS-IGA at Week 52 | responders |
| | Change from Baseline in total AN count at Week | Mean |
| | 52 | |
| | Change from Baseline in draining tunnel count at | Mean |
| | Week 52 | |
| | Responder status for remaining free from HS | Percentage of |
| | flares by Week 52 | responders |
| | Change from Baseline in IHS4 at Week 52 | Mean |
| | Change from Baseline on DLQI at Week 52 | Mean |
| | Change from Baseline in mean daily NRS for skin | Mean |
| | pain over Week 52 | |
| To assess the long-term | Adverse events over 52 weeks | Incidence per |
| safety and tolerability of | | patient year |
| brensocatib | Clinical laboratory test results, vital signs, and | Mean absolute |
| | ECGs over 52 weeks | value and mean |
| | | change from |
| | | Baseline |
| To evaluate the systemic | Brensocatib plasma concentrations over the | Descriptive |
| exposure of brensocatib | treatment period | statistics |

Exploratory
Period 1 (Double-Blind)

| | | |
|---|---|---|
| To evaluate the effect of | Time to achieve HiSCR in Period 1 | Hazard ratio |
| brensocatib compared | Responder status for achieving HiSCR, HiSCR75, | Odds ratio |
| with placebo | and HiSCR90 at each scheduled visit in Period 1 | |
| | Responder status for achieving mHiSCR at each | Odds ratio |
| | scheduled visit in Period 1 | |
| | Responder status for achieving ≥2-point decrease | Odds ratio |
| | from Baseline in HS-IGA at each scheduled visit | |
| | in Period 1 | |
| | Change from Baseline in total AN count at each | Mean difference |
| | scheduled visit in Period 1 | |
| | Change from Baseline in draining tunnel count at | Mean difference |
| | each scheduled visit in Period 1 | |
| | Responder status for remaining free from HS | Odds ratio |
| | flares at each scheduled visit in Period 1 | |
| | Change from Baseline in IHS4 at each scheduled | Mean difference |
| | visit in Period 1 | |
| | Change from Baseline in Hurley staging at each | Mean difference |
| | scheduled visit in Period 1 | |
| | Change from Baseline on DLQI at each scheduled | Mean difference |
| | visit in Period 1 | |
| | Average daily change from Baseline on NRS for | Mean difference |
| | skin pain over the week prior to each scheduled | |
| | visit in Period 1 | |
| | Average daily change from Baseline on HS- | Mean difference |
| | related PtGA over the week prior to each | |
| | scheduled visit in Period 1 | |
| | Change from Baseline on HiSQOL at each | Mean difference |
| | scheduled visit in Period 1 | |
| | Change from Baseline and absolute NSP activity | Descriptive |
| | (NE, CatG, PR3) in blood at individual time | statistics |
| | points in Period 1 | |

Period 2 (Open-Label)

| | | |
|---|---|---|
| To assess the long-term | Responder status for achieving HiSCR, HiSCR75, | Percentage of |
| effect of brensocatib | and HiSCR90 at each scheduled visit in Period 2 | responders |
| | Responder status for achieving mHiSCR at each | Percentage of |
| | scheduled visit in Period 2 | responders |
| | Responder status for achieving ≥2-point decrease | Percentage of |
| | from Baseline in HS-IGA at each scheduled visit | responders |
| | in Period 2 | |
| | Change from Baseline in total AN count at each | Mean |
| | scheduled visit in Period 2 | |
| | Change from Baseline in draining tunnel count at | Mean |
| | each scheduled visit in Period 2 | |
| | Responder status for remaining free from HS | Percentage of |
| | flares at each scheduled visit in Period 2 | responders |
| | Average daily change from Baseline on NRS for | Mean |
| | skin pain at each scheduled visit in Period 2 | |
| | Change from Baseline in IHS4 at each scheduled | Mean |
| | visit in Period 2 | |
| | Change from Baseline in Hurley staging at each | Mean |
| | scheduled visit in Period 2 | |
| | Change from Baseline on DLQI at each scheduled | Mean |
| | visit in Period 2 | |
| | Average daily change from Baseline on HS- | Mean |
| | related PtGA at each scheduled visit in Period 2 | |
| | Change from Baseline on HiSQOL at each | Mean |
| | scheduled visit in Period 2 | |
| | Change from Baseline and absolute NSP activity | Descriptive |
| | (NE, CatG, PR3) in blood at individual time | statistics |
| | points in Period 2 | |

Period 1 and Period 2

| To evaluate population | Plasma brensocatib concentration data from this study is used to develop | |
|---|---|---|
| PK of brensocatib | a population PK model to describe the PK of brensocatib in the HS | |
| | population using a non-linear mixed effect modeling approach. | |
| To explore PK/PD | Relationships between PK concentrations and | Based on PK-PD |
| relationships on safety, | response measures of efficacy (e.g., HiSCRs, HS- | relationships to |
| efficacy, and biomarkers | IGA, etc.), safety (AESIs), and blood NSP activity | identify optimal |
| | (NE, CatG, PR3) | doses and dosing |
| | | regimen for late |
| | | development |
| | | studies |
| To explore the effect of | Quantification of pro-inflammatory markers (e.g., | Mean absolute |
| brensocatib on tissue | TNF-α, IL-1β, IL-6) and NETs (e.g., MPO, Cit- | value and mean |
| samples from HS lesions | H4) in HS tissue samples at Week 16 and Week | change from |
| | 52 | Baseline |

^{a}HiSCR is defined as ≥50% reduction from Baseline in total AN count with no increase in abscess or draining tunnel count.
^{b}HiSCR75 is defined as ≥75% reduction from Baseline in total AN count with no increase in abscess or draining tunnel count.
^{c}HS flare is defined as ≥25% increase in AN count with an absolute increase in AN count of ≥2 relative to Baseline.
mHiSCR = modified Hidradenitis Suppurativa Clinical Response; NE = neutrophil elastase; NRS = numeric rating scale; NSP = neutrophil serine proteases; PtGA = Patient Global Assessment; PK/PD = pharmacokinetic/pharmacodynamic; PR3 = proteinase 3.

### Efficacy Assessments

### 1. HS Lesion Counts

HS lesions associated with each efficacy measure are presented in Table 4A. The number of abscesses, tunnels (draining and nondraining), and nodules (inflammatory and noninflammatory) are recorded by anatomical regions and used to programmatically derive the HiSCR, mHiSCR, HS-GA, total AN count, HS flare, and IHS4, as defined in Table 5B. The anatomical regions include both the upper body and the lower body. The upper body includes head, neck (anterior/posterior), chest/breast, axilla, abdomen, flank, and back. The lower body includes abdomen, back, inguinal, genital/perineal, gluteal (buttocks, perianal, cleft), hip, and thighs. The draining tunnel count is the sum of draining tunnels recorded in the HS lesion count assessment. While Hurley staging is based on an assessment of these same lesions, it is programmatically derived (see section 2).

TABLE 4A
HS Lesions Associated with Efficacy Parameters

| Efficacy | | Tunnel | | Nodule | |
|---|---|---|---|---|---|
| Parameter | Abscess | Draining | Nondraining | Inflammatory | Noninflammatory |
| HiSCR | X | X | | X | |
| mHiSCR | X | X | | X | |
| HS-IGA | X | X | X | X | X |
| Total AN count | X | | | X | |
| Draining tunnel | | X | | | |
| count | | | | | |
| HS flare | X | | | X | |
| IHS4 | X | X | | X | |
| Hurley staging | X | X | X | X | X |

TABLE 4B
Definitions of terms and efficacy parameters

| Term | Definition |
|---|---|
| Abscess | A tender, fluctuant (compressible), palpable lesion, with erythema |
| | (Frew et al., JAMA Dermatol. 2021; 157(4): 449-55) |
| Nodule | A solid, spherical, palpable lesion >1 cm |
| | Inflammatory nodule: tender and/or erythematous |
| | Noninflammatory nodule: nontender, nonerythematous (Frew et |
| | al., JAMA Dermatol. 2021; 157(4): 449-55) |
| Tunnel | A linear tract that may open onto the skin surface |
| | Draining tunnel: drainage expressed at rest or with compression |
| | of surrounding structures |
| | Nondraining tunnel: compression of surrounding structures does |
| | not elicit drainage of contents (Frew et al., JAMA Dermatol. |
| | 2021; 157(4): 449-55) |
| HiSCR | ≥50% reduction in total AN count, with no increase in abscess count |
| | and no increase in draining tunnel count relative to Baseline (Kimball |
| | et al., J Eur Acad Dermatol Venereol. 2016; 30(6): 989-94) |
| HiSCR75 | ≥75% reduction in total AN count, with no increase in abscess count |
| | and no increase in draining tunnel count relative to Baseline |
| HiSCR90 | ≥90% reduction in total AN count, with no increase in abscess count |
| | and no increase in draining tunnel count relative to Baseline |
| HS flare | ≥25% increase in AN count, with an absolute increase in AN count |
| | of ≥2 relative to Baseline |
| Hurley Stage | Stage I: Abscess formation, single or multiple, without sinus tracts |
| | and cicatrization |
| | Stage II: Recurrent abscesses with tract formation and |
| | cicatrization, single or multiple, widely separated lesions |
| | Stage III: Diffuse or near diffuse involvement, or multiple |
| | interconnected tracts and abscesses across the entire area (Hurley |
| | H., Dermatologic surgery. New York: Marcel Dekker. |
| | 1989; 729: 39). |
| mHiSCR | ≥50% reduction from Baseline in total number of inflammatory |
| | nodules, abscesses, and draining tunnels AND ≥50% reduction from |
| | Baseline in total number of draining tunnels |

### 1.1. Hidradenitis Suppurativa Clinical Response (HiSCR) and Modified HiSCR

HiSCR is a validated measure for assessing treatment response in the research setting, capturing the more acute phase of HS activity involving inflammatory changes. HiSCR is a clinical endpoint for measuring response to treatment in patients with HS and is defined by the status of 3 lesion types: abscesses, inflammatory nodules, and draining tunnels (Alikhan et al., J Am Acad Dermatol. 2019; 81(1):76-90; Kimball et al., Br J Dermatol. 2014; 171(6): 1434-42; Kimball et al., J Eur Acad Dermatol Venereol. 2016; 30(6):989-94). The HiSCR is programmatically derived from the HS lesion count assessments, with the response to treatment based on HiSCR, HiSCR75 and HiSCR90 relative to Day 1 (Baseline).

To assess the effect of treatment on reduction in draining tunnels, the HiSCR is modified to include a ≥50% reduction in the total number of draining tunnels resulting in the mHiSCR score. Thus, the response to treatment based on mHiSCR relative to Day 1 (Baseline) is defined as ≥50% reduction in number of inflammatory nodules, abscesses, and draining tunnels AND ≥50% reduction from Day 1 (Baseline) in total number of draining tunnels. The mHiSCR is programmatically derived from the HS lesion count assessments.

### 1.2. Hidradenitis Suppurativa—Investigator's Global Assessment (HS-IGA)

HS-IGA is a clinical endpoint for measuring disease activity and response to treatment in patients with HS that is based on the maximum count of abscesses, fistulas (draining and nondraining), and nodules (inflammatory and noninflammatory), measured separately for the upper body and the lower body regions (Garg et al., Br J Dermatol. 2022; 187(2):203-210). HS-IGA is scored as a number between 0 and 5 based on the maximum lesion count for either the upper body or the lower body regions, whichever is greater (Table 5). A response to treatment is defined as a 2-point reduction (improvement) in HS-IGA score relative to Baseline. Both regions are scored at each visit, and because the maximum count from either region is used to determine the HS-IGA score, the region used for scoring may differ from one visit to the next. The HS-IGA score is programmatically derived from the HS lesion count assessments.

TABLE 5
HS-IGA score

| HS-IGA | Lesion Count^{a} |
|---|---|
| 0 | 0-1 |
| 1 | 2-5 |
| 2 | 6-10 |
| 3 | 11-15 |
| 4 | 16-20 |
| 5 | >20 |

^{a}Sum of abscesses, nodules (inflammatory or noninflammatory) and fistulas (draining or nondraining) in either the upper body regions or the lower body regions, whichever is greater.

### 1.3. Total Abscess and Inflammatory Nodule Count and Draining Tunnel Count

The total AN count is defined by the total number of abscesses and inflammatory nodules. The total AN count is programmatically derived from the HS lesion count assessments.

### 1.4. HS Flare Assessment

An HS flare is defined as a ≥25% increase in total AN count with an absolute increase in total AN count of ≥2 relative to Baseline and is programmatically derived from the HS lesion count assessments. While there is no consensus on an objective and measurable definition of HS flare, this measure has been developed to approximate flare to the real-world clinical presentation and has been used in the pivotal Phase 3 PIONEER studies of adalimumab in HS (van der Zee et al., J Eur Acad Dermatol Venereol. 2020; 34(5):1050-56).

### 1.5. International Hidradenitis Suppurativa Severity Score 4 (IHS4)

IHS4 is a clinical scoring system to dynamically assess HS severity for use in both real-life and clinical study settings (Zouboulis et al., Br J Dermatol. 2017; 177(5):1401-09). The IHS4 includes lesions that are palpable with inflammatory signs (i.e., abscesses, nodules, and draining tunnels) and is complimentary to HiSCR.

The IHS4 score (points) is programmatically derived from the HS lesion count assessments and is calculated as follows: (number of inflammatory nodules×1)+(number of abscesses×2)+(number of draining tunnels×4).
Mild HS: ≤3 points
Moderate HS: 4 to 10 points
Severe HS: ≥11 points

### 2. Hurley Stage

The Hurley stage is a severity classification for HS that was developed in 1989 and is widely used for the determination of HS severity (Hurley, Dermatologic surgery. New York: Marcel Dekker. 1989; 729:39; Ovadja et al., Br J Dermatol. 2019; 181(2):344-49). The Hurley stage is determined in each affected anatomical region as defined above. If more than one stage is present in a region, the worst stage in each region is recorded.

### 3. Patient-Reported Outcome Measures

Patient-reported outcome measures selected for this study (i.e., DLQI, NRS for skin pain, HS related PtGA, and HiSQOL) are commonly used measures in HS clinical studies, with the DLQI, NRS skin pain, and HS-related PtGA being validated measures for their respective use in dermatologic diseases, atopic dermatitis, and HS (Basra et al., Br J Dermatol. 2008; 159(5):997-1035; Finlay et al., Clin Exp Dermatol. 1994; 19(3):210-6; Kirby et al., Br J Dermatol. 2021; 184(4):681-87; Newton et al., J Patient Rep Outcomes. 2019; 3(1):42). In this study, patient-reported outcome measures of DLQI, NRS for skin pain, HS-related PtGA, and HiSQOL are administered at designated study visits using an electronic device.

### 3.1. Dermatology Life Quality Index (DLQI)

DLQI is a self-administered questionnaire designed to measure the health related quality of life of adults (e.g., >16 years of age) suffering from a skin disease (Finlay et al., Clin Exp Dermatol. 1994; 19(3):210-6). It is comprised of 10 questions concerning the rater's perception of the impact of skin diseases on different aspects of the rater's quality of life over the past week. Each question has 4 alternative responses of not at all, a little, a lot, or very much, with corresponding scores of 0, 1, 2, and 3, respectively (a response of not relevant is scored as 0). The DLQI is the sum of scores with a range of 0 to 30. The higher the score, the greater the impairment in quality of life: 0-1=no effect at all; 2-5=small effect; 6-10=moderate effect; 11-20=very large effect; and 21-30=extremely large effect.

### 3.2. Numeric Rating Scale (NRS) for Skin Pain

NRS for skin pain is a single-item self-reported severity of worst skin pain on an 11-point scale of 0 (“no pain”) to 10 (“worst pain imaginable”) in the last 24 hours (Newton et al., J Patient Rep Outcomes. 2019; 3(1):42). Participants complete the NRS at the end of each day on an electronic device.

### 3.3. HS-Related Patient Global Assessment (HS-related PtGA)

HS-related PtGA is a single-item questionnaire assessing how much HS has influenced the participant's quality of life over the previous 7 days with 5 response levels of not at all, slightly, moderately, very much, and extremely and corresponding scores of 0, 1, 2, 3, and 4, respectively (Kirby et al., Br J Dermatol. 2021; 184(4):681-87).

### 3.4. Hidradenitis Suppurativa Quality of Life (HiSQOL)

HiSQOL is a valid and reliable measure of the impact of HS on health-related quality of life for use in clinical studies and has been shown to highly correlate with DLQI (Kirby et al., Br J Dermatol. 2020; 183(2):340-48; Kursawe Larsen et al., J Eur Acad Dermatol Venereol. 2021; 35(7):1577-81). The measured impacts include severity of HS-related symptoms (pain, itch, drainage, fatigue, odor), limitations in performance of activities (daily activities, physical, sleep), and psychosocial effects (emotional, cognitive, desire). HiSQOL is a 17-item instrument with a 7-day recall period. The 5-point item response scale of extremely, very much, moderately, slightly, or not at all corresponds to point values of 4, 3, 2, 1, and 0, respectively. Some items have an additional option of ‘unable to do so, due to my HS’ and/or ‘I do not normally do this, HS did not influence’ with assigned point values of 4 and 0, respectively.

### Pharmacokinetic (PK) Assessments

Blood samples of approximately 6 mL each are collected for measurement of plasma concentrations of brensocatib and for PK analysis. The timepoints for PK sample collections include:
pre-dose, 1 h (±5 min), 2 h (±10 min), and 6 h (±2 h) on Day 1;
1 h post-dose at Weeks 2, 6, and 12 during Period 1; and at Weeks 24, 32, and 52 during Period 2;
pre-dose at Weeks 4, 8, and 16 during Period 1.

Individual PK parameters (e.g., maximum plasma concentration (Cₘₐₓ), time to maximum observed plasma concentration (Tₘₐₓ), area under concentration-time curve from 0 to 24 hours post dose (AUC₂₄), elimination half-life (t/), apparent total clearance of drug from plasma after extravascular administration (CL/F), and apparent volume of distribution after terminal phase (Vd/F)) are evaluated using a population PK analysis. PK/PD relationships are also determined.

### Pharmacodynamic (PD) Assessments

### 1. PD Tissue Substudy

A PD tissue substudy is performed at select sites to characterize the effect of brensocatib on HS lesion pro-inflammatory markers and NETs. The substudy will include up to 30 evaluable participants (up to 10 per treatment arm) at select study sites from whom tissue samples are collected from active HS lesions via punch biopsies at on Day 1, Week 16 of Period 1, and Week 52 (End of Treatment) of Period 2. Punch biopsies are taken as close as possible to the previous biopsied site. An evaluable participant is considered one who receives at least 1 dose of study treatment and has at least 1 interpretable predose and 1 postdose tissue sample.

### 2. Biomarkers

Pharmacodynamic blood samples are collected to evaluate the effect of brensocatib compared with placebo and to evaluate the long-term effect of brensocatib on NSP activity in blood. Biomarkers will include NE, CatG, and PR3. Samples are collected at the following time points: Day 1; Weeks 2, 4, 6, 8, 12, and 16 during Period 1; Weeks 24, 32, and 52 (End of Treatment) of Period 2; and Week 56 (End of Study) of the follow-up period.

### Data Analyses

### Primary Efficacy Endpoint

The primary efficacy endpoint, responder status for achieving HiSCR at Week 16, is analyzed using a logistic regression in the Full Analysis Set (i.e., including all randomized participants). The model includes treatment group and randomization stratification factors of total IHS4 score (4-10 moderate vs >11 severe) and previous anti-TNF-α treatment (Yes vs No). The odds ratio for the treatment comparisons (brensocatib 10 mg vs placebo; brensocatib 40 mg vs placebo; each at two-sided α=0.05), the 95% Wald confidence intervals, and the corresponding p-value from the Wald chi-square test are summarized.

### Secondary Efficacy Endpoints

For Period 1, secondary variables assessed as changes from Baseline at Week 16 (i.e., total AN count, draining tunnel count, IHS4, and DLQI) or as mean daily changes over Week 16 (i.e., NRS for skin pain), are each analyzed using an analysis of covariance (ANCOVA) model with terms for treatment, stratification factors of total IHS4 score (4-10 moderate vs >11 severe) and previous anti-TNF-α treatment (Yes vs No), as well as a continuous fixed covariate for the corresponding baseline value. Secondary endpoints assessed as responder status at Week 16 (i.e., HiSCR75, achieving ≥2-point decrease from Baseline in HS-IGA, and for remaining free from HS flares) are each analyzed using a logistic regression adjusting for treatment group and randomization stratification factors of total IHS4 score (4-10 moderate vs >11 severe) and previous anti-TNF-α treatment (Yes vs No).

For Period 2, no inferential analyses are used. All analyses are descriptive. For the binary responder status endpoints at Week 52 (i.e., HiSCR, HiSCR75, achieving ≥2-point decrease from Baseline in HS-IGA, and for remaining free from HS flares), the frequency counts of responders are determined. For the continuous endpoints assessed as change from Baseline at Week 52 (i.e., total AN count, draining tunnel count, IHS4, and DLQI) or as mean daily changes over Week 52 (i.e., NRS for skin pain), descriptive statistics (n, mean, standard deviation (SD], median, minimum, and maximum) are determined.

For both Period 1 and Period 2, Baseline for the secondary endpoint change from Baseline in the mean daily NRS skin pain score over Week 16 or over Week 52 is the average of the daily NRS skin pain scores collected over the week prior to randomization. If there are ≥4 measurements collected within 7 days prior to randomization, Baseline will be the average of these measurements; if <4 measurements are collected, Baseline will be the average of the 4 most recent measurements prior to randomization.

### Safety Analyses

Safety analyses assess frequency and severity of treatment-emergent adverse events, including serious adverse events (SAEs) and AESIs, changes from Baseline in clinical laboratory test results, vital signs, and ECG. All safety analyses are based on descriptive statistics.

### PK Plasma Concentration Analyses

Plasma concentrations of brensocatib are summarized by treatment group and timepoint using descriptive statistics. Plasma concentration data versus time is plotted for individual participant and mean profiles on both linear and semi-logarithmic scales.

### Population Pharmacokinetics Analyses

Plasma brensocatib concentration data from this study is used to develop a population PK model to describe the PK of brensocatib in the HS population using a non-linear mixed effect modeling approach. In this analysis, covariates (e.g., weight, age, sex, creatinine clearance, etc.) on brensocatib PK are evaluated. The final population PK model is used to predict individual PK parameters (e.g., Cₘₐₓ, Tₘₐₓ, AUC₂₄, elimination t_{1/2}, CL/F, and Vd/F).

### Pharmacodynamic Analyses

Blood NSP activity (NE, CatG, PR3) is used to determine the change from Baseline and percentage of inhibition over time using baseline activity as a reference. The relationship between NSP inhibition and dose level is explored along with the relationship between NSP inhibition and clinical effect.

### Pharmacokinetic (PK)/Pharmacodynamic (PD) Analyses

Using the predicted brensocatib exposure from the final population PK model, the relationships between brensocatib exposure (e.g., AUC, Cₘₐₓ, etc.) and clinical efficacy (e.g., HiSCR, HiSCR75, HiSCR90, HS-IGA, etc.), safety (e.g., AESIs, etc.), or blood NSP activity (e.g., NE, CatG, PR3) are assessed using statistical methods or modeling approach. Based on the PK-PD relationships, optimal brensocatib exposure and dose(s)/regimen(s) for treatment of patients with HS are identified via simulation.

All papers, publications and patents cited in this specification are herein incorporated by reference as if each individual paper, publication or patent were specifically and individually indicated to be incorporated by reference and are incorporated herein by reference to disclose and describe the methods and/or materials in connection with which the publications are cited. However, mention of any reference, article, publication, patent, patent publication, and patent application cited herein is not, and should not be taken as an acknowledgment or any form of suggestion that they constitute valid prior art or form part of the common general knowledge in any country in the world.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. A method of treating hidradenitis suppurativa (HS) in a subject in need thereof, comprising: administering to the subject for an administration period, a pharmaceutical composition comprising an effective amount of a compound of Formula (I), or a pharmaceutically acceptable salt thereof, 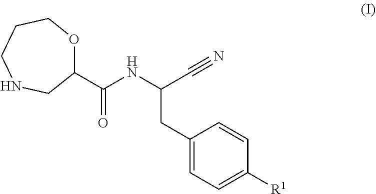
wherein,
R¹ is 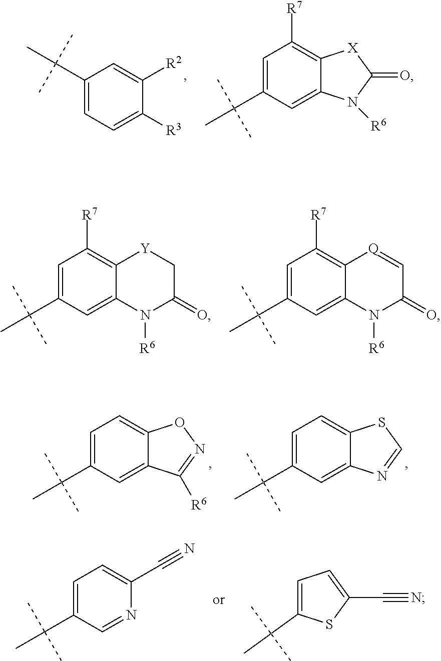
R² is hydrogen, F, Cl, Br, OSO₂C₁₋₃alkyl, or C₁₋₃alkyl;
R³ is hydrogen, F, Cl, Br, CN, CF₃, SO₂C₁₋₃alkyl, CONH₂ or SO₂NR⁴R⁵,
wherein R⁴ and R⁵ together with the nitrogen atom to which they are attached form an azetidine, pyrrolidine or piperidine ring; or
X is O, S or CF₂;
Y is O or S;
Q is CH or N;
R⁶ is C₁₋₃alkyl, wherein said C₁₋₃alkyl is optionally substituted by 1, 2 or 3 F and optionally by one substituent selected from the group consisting of OH, OC₁₋₃alkyl, N(C₁₋₃alkyl)₂, cyclopropyl, and tetrahydropyran; and
R⁷ is hydrogen, F, Cl or CH₃,
thereby treating hidradenitis suppurativa in the subject.

2. The method of claim 1, wherein the subject has not been treated with an anti-TNFα agent previously.

3. The method of claim 1 or 2, wherein the HS is Hurley Stage I HS, Hurley Stage II HS or Hurley Stage III HS.

4. The method of claim 3, wherein the HS is Hurley Stage I HS.

5. The method of claim 3, wherein the HS is Hurley Stage II HS.

6. The method of claim 3, wherein the HS is Hurley Stage III HS.

7. The method of any one of claims 1-6, wherein the compound of Formula (I) inhibits DPP1.

8. The method of any one of claims 1-7, wherein the HS is refractory HS.

9. The method of claim 8, wherein the HS was not responsive to a prior, different HS treatment.

10. The method of claim 9, wherein the prior, different HS treatment comprises administration of a systemic antibiotic.

11. The method of any one of claims 1-10, further comprising diminishing the severity of, or eliminating one or more symptoms of HS of the subject, during or subsequent to the administration period, compared to the one or more symptoms prior to the administration period.

12. The method of claim 11, wherein the one or more symptoms of HS are: (a) one or more skin lesions; (b) one or more skin abscesses; (c) one or more fistulas; (d) one or more sinus tracts; (e) skin scarring; (f) anxiety; (g) depression; (h) suicidal thoughts; or (i) any combinations thereof.

13. The method of claim 12, wherein prior to the administration period, the one or more skin lesions, one or more skin abscesses, one or more fistulas, one or more sinus tracts, or a combination thereof are painful, inflamed, swollen, or a combination thereof.

14. The method of claim 12 or claim 13, wherein the one or more skin lesions, one or more skin abscesses, one or more fistulas, one or more sinus tracts, or a combination thereof are present on an armpit, groin, buttocks, or a combination thereof, of the subject.

15. The method of any one of claims 12-14, wherein the one or more skin lesions, one or more skin abscesses, one or more fistulas, one or more sinus tracts, or a combination thereof are associated with hair follicles.

16. The method of any one of claims 12-15, wherein the one or more skin lesions are one or more skin nodules.

17. The method of claim 16, wherein the one or more skin nodules are one or more inflammatory skin nodules.

18. The method of any one of claims 12-17, wherein the one or more skin lesions are interconnected by one or more sinus tracts.

19. The method of any one of claims 12-18, wherein the one or more skin lesions are one or more fistulas.

20. The method of claim 19, wherein the one or more fistulas comprise one or more draining fistulas.

21. The method of any one of claims 1-20, further comprising reducing a skin abscess and inflammatory skin nodule count (AN count) of the subject during or subsequent to the administration period, as compared to an AN count of the subject prior to the administration period.

22. The method of claim 21, wherein the AN count of the subject prior to the administration period is ≥3.

23. The method of claim 21 or 22, wherein the AN count of the subject during or subsequent to the administration period is at least 10% less than the AN count of the subject prior to the administration period.

24. The method of any one of claims 1-23, comprising reducing an occurrence of HS flares in the subject during or subsequent to the administration period, as compared to an occurrence of HS flares in the subject prior to the administration period.

25. The method of any one of claims 1-24, comprising reducing or maintaining a fistula count of the subject during or subsequent to the administration period, as compared to a fistula count of the subject prior to the administration period.

26. The method of claim 25, wherein the fistula count of the subject prior to the administration period is ≤20.

27. The method of claim 25 or 26, wherein the fistula count is a draining fistula count.

28. The method of any one of claims 1-27, wherein the subject achieves a Hidradenitis Suppurative Clinical Response 50 (HiSCR50) during or subsequent to the administration period, wherein the HiSCR50 is at least a 50% reduction in total AN count, with no increase in skin abscess count, and no increase in draining fistula count, as compared to a total AN count, a skin abscess count, and a draining fistula count of the subject, respectively, prior to the administration period.

29. The method of any one of claims 1-28, wherein the subject achieves a Hidrahenitis Suppurative Clinical Response 75 (HiSCR75) during or subsequent to the administration period, wherein the HiSCR75 is at least a 75% reduction in total AN count, with no increase in skin abscess count, and no increase in draining fistula count, as compared to a total AN count, a skin abscess count, and a draining fistula count of the subject, respectively, prior to the administration period.

30. The method of any one of claims 1-29, wherein the subject achieves a Hidrahenitis Suppurative Clinical Response 90 (HiSCR90) during or subsequent to the administration period, wherein the HiSCR90 is at least a 90% reduction in total AN count, with no increase in skin abscess count, and no increase in draining fistula count, as compared to a total AN count, a skin abscess count, and a draining fistula count of the subject, respectively, prior to the administration period.

31. The method of any one of claims 1-30, wherein the subject achieves a modified Hidradenitis Suppurativa Clinical Response (mHiSCR) during or subsequent to the administration period, wherein the mHiSCR is a ≥50% reduction in total number of inflammatory nodules, abscesses, and draining fistulas and a ≥50% reduction in total number of draining fistulas, as compared to a total number of inflammatory nodules, abscesses, and draining fistulas and a total number of draining fistulas of the subject, respectively, prior to the administration period.

32. The method of any one of claims 1-31, comprising reducing a Patient's Global Assessment of Skin Pain (PGA-SP) score of the subject during or subsequent to the administration period, as compared to a PGA-SP score of the subject prior to the administration period.

33. The method of claim 32, wherein the PGA-SP score of the subject prior to the administration period is ≥3.

34. The method of claim 32 or 33, wherein the PGA-SP score of the subject during or subsequent to the administration period is at least about 10% less than the PGA-SP score of the subject prior to the administration period.

35. The method of any one of claims 32-34, wherein the PGA-SP score of the subject during or subsequent to the administration period is 3 or less.

36. The method of any one of claims 1-35, comprising reducing an International hidradenitis suppurativa severity (IHS4) score of the subject during or subsequent to the administration period, as compared to an IHS4 score of the subject prior to the administration period.

37. The method of claim 36, wherein the IHS4 score of the subject during or subsequent to the administration period is at least about 10% less than the IHS4 score of the subject prior to the administration period.

38. The method of claim 36 or 37, wherein the IHS4 score of the subject during or subsequent to the administration period is reduced from an HS Hurley Stage III score to an HS Hurley Stage II score.

39. The method of claim 36 or 37, wherein the IHS4 score of the subject during or subsequent to the administration period is reduced from an HS Hurley Stage III score to an HS Hurley Stage I score.

40. The method of claim 36 or 37, wherein the IHS4 score of the subject during or subsequent to the administration period is reduced from an HS Hurley Stage II score to an HS Hurley Stage I score.

41. The method of any one of claims 36-40, wherein the IHS4 score of the subject is ≥4 prior to the administration period.

42. The method of claim 41, wherein the IHS4 score of the subject is 10 prior to the administration period.

43. The method of claim 41, wherein the IHS4 score of the subject is 11 prior to the administration period.

44. The method of any one of claims 1-43, comprising reducing a Hidradenitis Suppurativa Quality of Life (HiSQOL) score of the subject during or subsequent to the administration period, as compared to a HiSQOL score of the subject prior to the administration period.

45. The method of any one of claims 1-44, comprising reducing a Hidradenitis Suppurativa-Investigator's Global Assessment (HS-IGA) score of the subject during or subsequent to the administration period, as compared to an HS-IGA score of the subject prior to the administration period.

46. The method of claim 45, wherein the HS-IGA score of the subject is reduced ≥2 points during or subsequent to the administration period.

47. The method of any one of claims 1-46, comprising reducing a Dermatology Life Quality Index (DLQI) score of the subject during or subsequent to the administration period, as compared to a DLQI score of the subject prior to the administration period.

48. The method of any one of claims 1-47, comprising improving the Hurley Stage of the HS of the subject during or subsequent to the administration period, as compared to the Hurley Stage of the HS of the subject prior to the administration period.

49. The method of claim 48, wherein the improving the Hurley Stage of the HS of the subject comprises reducing the severity of the HS of the subject from Hurley Stage III to Hurley Stage II.

50. The method of claim 48, wherein the improving the Hurley Stage of the HS of the subject comprises reducing the severity of the HS of the subject from Hurley Stage III to Hurley Stage I.

51. The method of claim 48, wherein the improving the Hurley Stage of the HS of the subject comprises reducing the severity of the HS of the subject from Hurley Stage II to Hurley Stage I.

52. The method of any one of claims 1-51, comprising reducing a HS-Related Patient Global Assessment (HS-related PtGA) score of the subject during or subsequent to the administration period, as compared to an HS-related PtGA score of the subject prior to the administration period.

53. The method of any one of claims 1-52, wherein the number of neutrophils in a biological sample obtained from the subject prior to the administration period is greater than the number of neutrophils in a counterpart biological sample obtained from a control subject who does not have HS.

54. The method of any one of claims 1-53, wherein the number of neutrophils in a biological sample obtained from the subject during or subsequent to the administration period is less than: (a) the number of neutrophils in a counterpart biological sample obtained from the subject prior to the administration period, and/or (b) the number of neutrophils in a counterpart biological sample obtained from a control subject, wherein the control subject has HS and is not administered the pharmaceutical composition.

55. The method of any one of claims 1-54, wherein the level of SA100A7 in a biological sample obtained from the subject prior to the administration period is greater than the level of SA100A7 in a counterpart biological sample obtained from a control subject who does not have HS.

56. The method of any one of claims 1-55, wherein the level of SA100A7 in a biological sample obtained from the subject during or subsequent to the administration period is less than: (a) the level of SA100A7 in a counterpart biological sample obtained from the subject prior to the administration period, and/or (b) the level of SA100A7 in a counterpart biological sample obtained from a control subject, wherein the control subject has HS and is not administered the pharmaceutical composition.

57. The method of any one of claims 1-56, wherein the level of myeloperoxidase (MPO) in a biological sample obtained from the subject prior to the administration period is greater than the level of MPO in a counterpart biological sample obtained from a control subject who does not have HS.

58. The method of any one of claims 1-57, wherein the level of MPO in a biological sample obtained from the subject during or subsequent to the administration period is less than: (a) the level of MPO in a counterpart biological sample obtained from the subject prior to the administration period, and/or (b) the level of MPO in a counterpart biological sample obtained from a control subject, wherein the control subject has HS and is not administered the pharmaceutical composition.

59. The method of any one of claims 1-58, wherein the activity of peptidylarginine deiminase 4 (PAD4) in a biological sample obtained from the subject prior to the administration period is greater than the activity of peptidylarginine deiminase 4 (PAD4) in a counterpart biological sample obtained from a control subject who does not have HS.

60. The method of any one of claims 1-59, wherein the level of PAD4 in a biological sample obtained from the subject during or subsequent to the administration period is less than: (a) the level of PAD4 in a counterpart biological sample obtained from the subject prior to the administration period, and/or (b) the level of PAD4 in a counterpart biological sample obtained from a control subject, wherein the control subject has HS and is not administered the pharmaceutical composition.

61. The method of any one of claims 1-60, wherein the level of a cytokine and/or a lipid mediator in a biological sample obtained from the subject prior to the administration period is greater than the level of the cytokine and/or the lipid mediator in a counterpart biological sample obtained from a control subject who does not have HS.

62. The method of any one of claims 1-61, wherein the level of a cytokine and/or lipid mediator in a biological sample obtained from the subject during or subsequent to the administration period is less than: (a) the level of the cytokine and/or lipid mediator in a counterpart biological sample obtained from the subject prior to the administration period, and/or (b) the level of the cytokine and/or lipid mediator in a counterpart biological sample obtained from a control subject, wherein the control subject has HS and is not administered the pharmaceutical composition.

63. The method of claim 61 or 62, wherein the cytokine is a chemokine.

64. The method of any one of claims 1-63, wherein the level of C-reactive protein (CRP) in a biological sample obtained from the subject prior to the administration period is greater than the level of C-reactive protein (CRP) in a counterpart biological sample obtained from a control subject who does not have HS.

65. The method of any one of claims 1-64, wherein the level of CRP in a biological sample obtained from the subject during or subsequent to the administration period is less than: (a) the level of CRP in a counterpart biological sample obtained from the subject prior to the administration period, and/or (b) the level of CRP in a counterpart biological sample obtained from a control subject, wherein the control subject has HS and is not administered the pharmaceutical composition.

66. The method of any one of claims 1-65, wherein prior to the administration period, the level of DPP1 in a biological sample obtained from the subject is in the range of from about 1 ng/mL to about 100 ng/mL.

67. The method of any one of claims 1-66, wherein prior to the administration period, the level of neutrophil extracellular traps (NETs) in a biological sample obtained from the subject is in the range of from about 1 ng/mL to about 1000 ng/mL.

68. The method of any one of claims 1-67, wherein prior to the administration period, the level of a neutrophil serine protease (NSP) in a biological sample obtained from the subject is in the range of from about 1 ng/mL to about 1000 ng/mL.

69. The method of claim 68, wherein the NSP is neutrophil elastase (NE), proteinase 3 (PR3), cathepsin G (CatG), neutrophil serine protease 4 (NSP4), or any combination thereof.

70. The method of any one of claims 1-69, wherein the activity of DPP1 in a biological sample obtained from the subject during or subsequent to the administration period is less than: (a) the activity of DPP1 in a counterpart biological sample obtained from the subject prior to the administration period, and/or (b) the activity of DPP1 in a counterpart biological sample obtained from a control subject, wherein the control subject has HS and is not administered the pharmaceutical composition.

71. The method of any one of claims 1-70, wherein the activity of a neutrophil serine protease (NSP) in a biological sample obtained from the subject during or subsequent to the administration period is less than: (a) the activity of the NSP in a counterpart biological sample obtained from the subject prior to the administration period, and/or (b) the activity of the NSP in a counterpart biological sample obtained from a control subject, wherein the control subject has HS and is not administered the pharmaceutical composition.

72. The method of claim 71, wherein the NSP is selected from the group consisting of neutrophil elastase (NE), proteinase 3 (PR3), cathepsin G (CatG), and neutrophil serine protease 4 (NSP4).

73. The method of any one of claims 1-72, wherein the level of neutrophil extracellular traps (NETs) in a biological sample obtained from the subject during or subsequent to the administration period is less than: (a) the level of NETs in a counterpart biological sample obtained from the subject prior to the administration period, and/or (b) the level of NETs in a counterpart biological sample obtained from a control subject, wherein the control subject has HS and is not administered the pharmaceutical composition.

74. The method of any one of claims 53-73, wherein the biological sample is skin tissue, blood, serum, or a combination thereof.

75. The method of claim 74, wherein the biological sample is skin tissue.

76. The method of claim 74 or claim 75, wherein the skin tissue comprises tissue derived from one or more skin lesions, one or more skin abscesses, one or more fistulas, one or more sinus tracts, one or more skin scars, one or more sinus tracts, or a combination thereof.

77. The method of claim 76, wherein the skin tissue comprises tissue derived from one or more fistulas.

78. The method of claim 77, wherein the one or more fistulas comprises one or more draining fistulas.

79. The method of any one of claims 1-78, further comprising administering a secondary therapy to the subject.

80. The method of claim 79, wherein the secondary therapy is administered: (a) prior to the administration period, (b) during or subsequent to the administration period, or (c) concurrently with administering the pharmaceutical composition.

81. The method of claim 79 or claim 80, wherein the secondary therapy comprises one or more of the following: an antibiotic, a steroid, a biologic, a hormone, a retinoid, an anti-inflammatory drug, a surgical intervention, or any combination thereof.

82. The method of claim 81, wherein the antibiotic is applied topically.

83. The method of claim 81 or claim 82, wherein the antibiotic is doxycycline, minocycline, clindamycin, rifampin, or any combination thereof.

84. The method of claim 81, wherein the steroid is triamcinolone.

85. The method of claim 81, wherein the biologic is an anti-TNF-alpha antibody.

86. The method of claim 85, wherein the anti-TNF-alpha antibody is adalimumab, infliximab, or a combination thereof.

87. The method of claim 81, wherein the biologic is an IL-17 inhibitor, an IL-12 inhibitor, an IL-23 inhibitor, a JAK-1 inhibitor, or any combination thereof.

88. The method of claim 81, wherein the hormone is estrogen.

89. The method of claim 81, wherein the anti-inflammatory drug is aspirin, ibuprofen, naproxen, celecoxib, indomethacin, ketorolac, diclofenac, ketoprofen, or any combination thereof.

90. The method of any one of claims 1-89, wherein the compound of Formula (I), or a pharmaceutically acceptable salt thereof, is the S,S diastereomer:

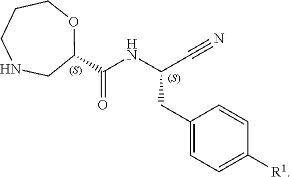

91. The method of any one of claims 1-89, wherein the compound of Formula (I), or a pharmaceutically acceptable salt thereof, is the S,R diastereomer:

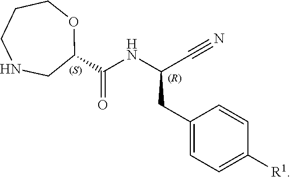

92. The method of any one of claims 1-89, wherein the compound of Formula (I), or a pharmaceutically acceptable salt thereof, is the R,S diastereomer:

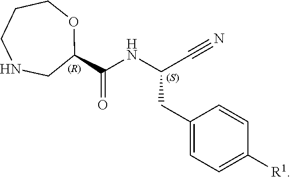

93. The method of any one of claims 1-89, wherein the compound of Formula (I), or a pharmaceutically acceptable salt thereof, is the R,R diastereomer:

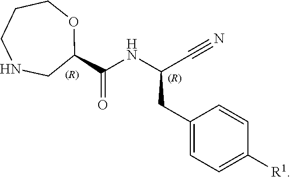

94. The method of any one of claims 1-93, wherein,
R¹ is 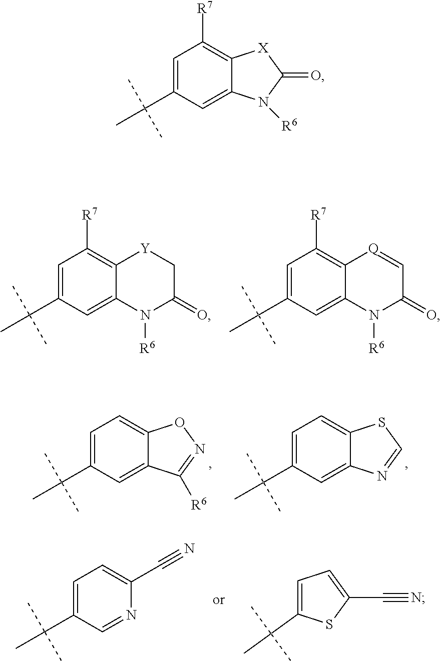
X is O, S or CF₂;
Y is O or S;
Q is CH or N;
R⁶ is C₁₋₃alkyl, wherein said C₁₋₃alkyl is optionally substituted by 1, 2 or 3 F and optionally by one substituent selected from the group consisting of OH, OC₁₋₃alkyl, N(C₁₋₃alkyl)₂, cyclopropyl, and tetrahydropyran; and
R⁷ is hydrogen, F, Cl or CH₃.

95. The method of any one of claims 1-94, wherein,
R¹ is 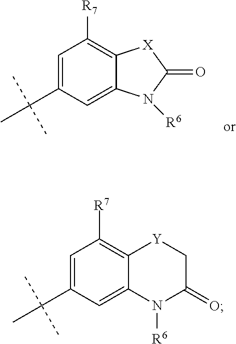
X is O, S or CF₂;
Y is O or S;
R⁶ is C₁₋₃alkyl, wherein said C₁₋₃alkyl is optionally substituted by 1, 2 or 3 F and optionally by one substituent selected from the group consisting of OH, OC₁₋₃alkyl, N(C₁₋₃alkyl)₂, cyclopropyl, and tetrahydropyran; and
R⁷ is hydrogen, F, Cl or CH₃.

96. The method of any one of claims 1-95, wherein, R¹ is

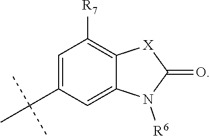

97. The method of claim 96, wherein X is O; R⁶ is C₁₋₃alkyl; and R⁷ is hydrogen.

98. The method of any one of claims 1-96, wherein,
R¹ is 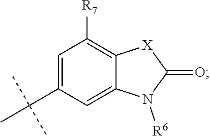
X is O;
R⁶ is C₁₋₃alkyl, wherein said C₁₋₃alkyl is optionally substituted by 1, 2 or 3 F; and
R⁷ is hydrogen.

99. The method of any one of claims 1-96, wherein,
R¹ is 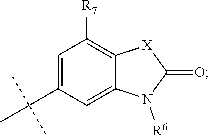
X is O;
R⁶ is C₁₋₃alkyl; and
R⁷ is hydrogen.

100. The method of claim 90, wherein the compound of Formula (I) is (2S)—N-{(1S)-1-cyano-2-[4-(3-methyl-2-oxo-2,3-dihydro-1,3-benzoxazol-5-y-l)phenyl]ethyl}-1,4-oxazepane-2-carboxamide (brensocatib) 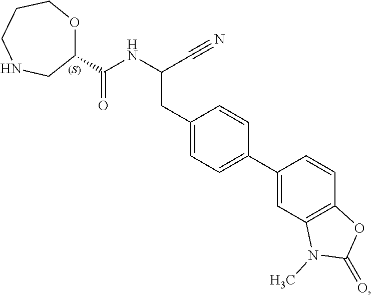
or a pharmaceutically acceptable salt thereof.

101. The method of claim 90, wherein the compound of Formula (I) is (2S)—N-{(1S)-1-cyano-2-[4-(3-methyl-2-oxo-2,3-dihydro-1,3-benzoxazol-5-y-l)phenyl]ethyl}-1,4-oxazepane-2-carboxamide (brensocatib)

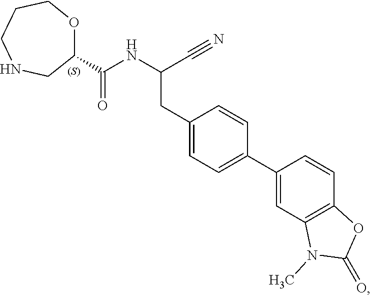

102. The method of claim 90, wherein the compound of Formula (I) is a hydrate of (2S)—N-{(1S)-1-cyano-2-[4-(3-methyl-2-oxo-2,3-dihydro-1,3-benzoxazol-5-y-l)phenyl]ethyl}-1,4-oxazepane-2-carboxamide (brensocatib) 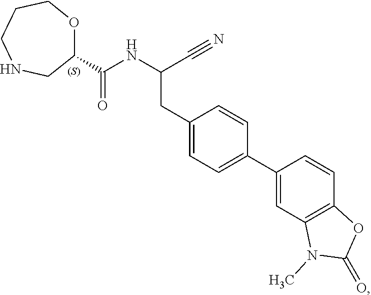
or a pharmaceutically acceptable salt thereof.

103. The method of claim 90, wherein the compound of Formula (I) is selected from the group consisting of
(2S)—N-[(1S)-1-Cyano-2-(4′-cyanobiphenyl-4-yl)ethyl]-1,4-oxazepane-2-carboxamide;
(2S)—N-{(1S)-1-Cyano-2-[4-(3-methyl-2-oxo-2,3-dihydro-1,3-benzoxazol-5-yl)phenyl]ethyl}-1,4-oxazepane-2-carboxamide;
(2S)—N-{(1S)-1-Cyano-2-[4-(3,7-dimethyl-2-oxo-2,3-dihydro-1,3-benzoxazol-5-yl)phenyl]ethyl}-1,4-oxazepane-2-carboxamide;
4′-[(2S)-2-Cyano-2-{[(2S)-1,4-oxazepan-2-ylcarbonyl]amino}ethyl]biphenyl-3-yl methanesulfonate;
(2S)—N-{(1S)-1-Cyano-2-[4-(3-methyl-1,2-benzoxazol-5-yl)phenyl]ethyl}-1,4-oxazepane-2-carboxamide;
(2S)—N-{(1S)-1-Cyano-2-[4′-(trifluoromethyl)biphenyl-4-yl]ethyl}-1,4-oxazepane-2-carboxamide;
(2S)—N-[(1S)-1-Cyano-2-(3′,4′-difluorobiphenyl-4-yl)ethyl]-1,4-oxazepane-2-carboxamide;
(2S)—N-{(15)-1-Cyano-2-[4-(6-cyanopyridin-3-yl)phenyl]ethyl}-1,4-oxazepane-2-carboxamide;
(2S)—N-{(1S)-1-Cyano-2-[4-(4-methyl-3-oxo-3,4-dihydro-2H-1,4-benzothiazin-6-yl)phenyl]ethyl}-1,4-oxazepane-2-carboxamide;
(2S)—N-{(1S)-1-Cyano-2-[4-(3-ethyl-7-methyl-2-oxo-2,3-dihydro-1,3-benzoxazol-5-yl)phenyl]ethyl}-1,4-oxazepane-2-carboxamide;
(2S)—N-[(1S)-1-Cyano-2-{4-[3-(2-hydroxy-2-methylpropyl)-2-oxo-2,3-dihydro-1,3-benzoxazol-5-yl]phenyl}ethyl]-1,4-oxazepane-2-carboxamide;
(2S)—N-[(1S)-1-Cyano-2-{4-[3-(2,2-difluoroethyl)-7-fluoro-2-oxo-2,3-dihydro-1,3-benzoxazol-5-yl]phenyl}ethyl]-1,4-oxazepane-2-carboxamide;
(2S)—N-[(1S)-1-Cyano-2-(4-{3-[2-(dimethylamino)ethyl]-2-oxo-2,3-dihydro-1,3-benzoxazol-5-yl}phenyl)ethyl]-1,4-oxazepane-2-carboxamide;
(2S)—N-{(1S)-1-Cyano-2-[4-(3,3-difluoro-1-methyl-2-oxo-2,3-dihydro-1H-indol-6-yl)phenyl]ethyl}-1,4-oxazepane-2-carboxamide;
(2S)—N-{(15)-1-Cyano-2-[4-(7-fluoro-3-methyl-2-oxo-2,3-dihydro-1,3-benzoxazol-5-yl)phenyl]ethyl}-1,4-oxazepane-2-carboxamide;
(2S)—N-{(15)-1-Cyano-2-[4-(3-ethyl-2-oxo-2,3-dihydro-1,3-benzoxazol-5-yl)phenyl]ethyl}-1,4-oxazepane-2-carboxamide;
(2S)—N-[(1S)-1-Cyano-2-{4-[3-(cyclopropylmethyl)-2-oxo-2,3-dihydro-1,3-benzoxazol-5-yl]phenyl}ethyl]-1,4-oxazepane-2-carboxamide;
(2S)—N-[(1S)-1-Cyano-2-{4-[3-(2-methoxyethyl)-2-oxo-2,3-dihydro-1,3-benzothiazol-5-yl]phenyl}ethyl]-1,4-oxazepane-2-carboxamide;
(2S)—N-[(1S)-1-Cyano-2-{4-[2-oxo-3-(propan-2-yl)-2,3-dihydro-1,3-benzoxazol-5-yl]phenyl}ethyl]-1,4-oxazepane-2-carboxamide;
(2S)—N-{(15)-1-Cyano-2-[4-(4-methyl-3-oxo-3,4-dihydro-2H-1,4-benzoxazin-6-yl)phenyl]ethyl}-1,4-oxazepane-2-carboxamide;
(2S)—N-[(1S)-1-Cyano-2-{4-[3-(2-methoxyethyl)-2-oxo-2,3-dihydro-1,3-benzoxazol-5-yl]phenyl}ethyl]-1,4-oxazepane-2-carboxamide;
(2S)—N-{(1S)-1-Cyano-2-[4-(5-cyanothiophen-2-yl)phenyl]ethyl}-1,4-oxazepane-2-carboxamide;
(2S)—N-[(1S)-2-(4′-Carbamoyl-3′-fluorobiphenyl-4-yl)-1-cyanoethyl]-1,4-oxazepane-2-carboxamide;
(2S)—N-{(1S)-1-Cyano-2-[4-(1-methyl-2-oxo-1,2-dihydroquinolin-7-yl)phenyl]ethyl}-1,4-oxazepane-2-carboxamide;
(2S)—N-[(1S)-1-Cyano-2-{4-[2-oxo-3-(tetrahydro-2H-pyran-4-ylmethyl)-2,3-dihydro-1,3-benzoxazol-5-yl]phenyl}ethyl]-1,4-oxazepane-2-carboxamide;
(2S)—N-{(1S)-2-[4-(7-Chloro-3-methyl-2-oxo-2,3-dihydro-1,3-benzoxazol-5-yl)phenyl]-1-cyanoethyl}-1,4-oxazepane-2-carboxamide;
(2S)—N-[(1S)-1-Cyano-2-{4-[3-(2,2-difluoroethyl)-2-oxo-2,3-dihydro-1,3-benzoxazol-5-yl]phenyl}ethyl]-1,4-oxazepane-2-carboxamide;
(2S)—N-[(1S)-1-Cyano-2-{4-[2-oxo-3-(2,2,2-trifluoroethyl)-2,3-dihydro-1,3-benzoxazol-5-yl]phenyl}ethyl]-1,4-oxazepane-2-carboxamide;
(2S)—N-{(1S)-1-Cyano-2-[4-(3-methyl-2-oxo-2,3-dihydro-1,3-benzothiazol-5-yl)phenyl]ethyl}-1,4-oxazepane-2-carboxamide;
(2S)—N-{(1S)-1-Cyano-2-[4′-(methylsulfonyl)biphenyl-4-yl]ethyl}-1,4-oxazepane-2-carboxamide;
(2S)—N-{(1S)-2-[4′-(Azetidin-1-ylsulfonyl)biphenyl-4-yl]-1-cyanoethyl}-1,4-oxazepane-2-carboxamide;
(2S)—N-[(1S)-1-Cyano-2-(4′-fluorobiphenyl-4-yl)ethyl]-1,4-oxazepane-2-carboxamide;
(2S)—N-{(1S)-2-[4-(1,3-Benzothiazol-5-yl)phenyl]-1-cyanoethyl}-1,4-oxazepane-2-carboxamide;
(2S)—N-[(1S)-1-Cyano-2-(4′-cyanobiphenyl-4-yl)ethyl]-1,4-oxazepane-2-carboxamide;
and pharmaceutically acceptable salts thereof.

104. The method of claim 91, wherein the compound of Formula (I) is (2S)—N-{(1R)-1-Cyano-2-[4-(3-methyl-2-oxo-2,3-dihydro-1,3-benzoxazol-5-yl)phenyl]ethyl}-1,4-oxazepane-2-carboxamide: 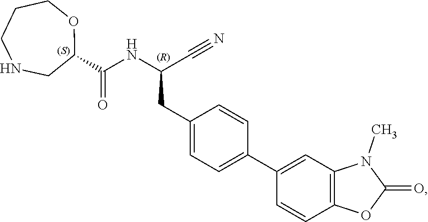
or a pharmaceutically acceptable salt thereof.

105. The method of claim 104, wherein the compound of Formula (I) is (2S)—N-{(1R)-1-Cyano-2-[4-(3-methyl-2-oxo-2,3-dihydro-1,3-benzoxazol-5-yl)phenyl]ethyl}-1,4-oxazepane-2-carboxamide:

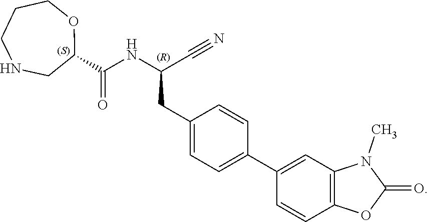

106. The method of claim 92, wherein the compound of Formula (I) is (2R)—N-{(1S)-1-Cyano-2-[4-(3-methyl-2-oxo-2,3-dihydro-1,3-benzoxazol-5-yl)phenyl]ethyl}-1,4-oxazepane-2-carboxamide: 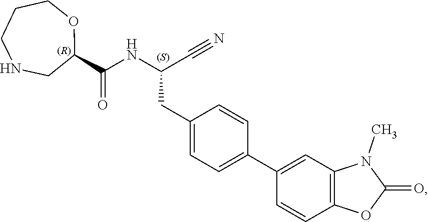
or a pharmaceutically acceptable salt thereof.

107. The method of claim 106, wherein the compound of Formula (I) is (2R)—N-{(1S)-1-Cyano-2-[4-(3-methyl-2-oxo-2,3-dihydro-1,3-benzoxazol-5-yl)phenyl]ethyl}-1,4-oxazepane-2-carboxamide:

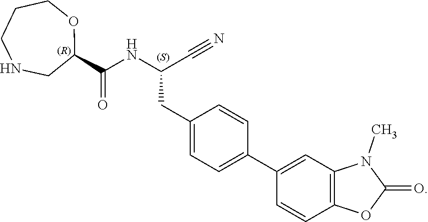

108. The method of claim 93, wherein the compound of Formula (I) is (2R)—N-{(1R)-1-Cyano-2-[4-(3-methyl-2-oxo-2,3-dihydro-1,3-benzoxazol-5-yl)phenyl]ethyl}-1,4-oxazepane-2-carboxamide: 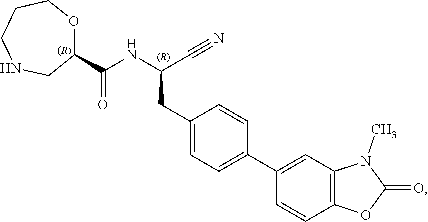
or a pharmaceutically acceptable salt thereof.

109. The method of claim 108, wherein the compound of Formula (I) is (2R)—N-{(1R)-1-Cyano-2-[4-(3-methyl-2-oxo-2,3-dihydro-1,3-benzoxazol-5-yl)phenyl]ethyl}-1,4-oxazepane-2-carboxamide:

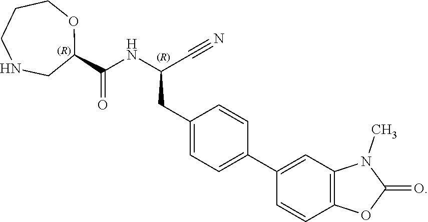

110. The method of any one of claims 1-89, wherein the pharmaceutical composition comprises a mixture of an S,S diastereomer of a compound of Formula (I) and an S,R diastereomer of a compound of Formula (I).

111. The method of any one of claims 1-89, wherein the pharmaceutical composition comprises a mixture of an S,S diastereomer of a compound of Formula (I) and an R,S diastereomer of a compound of Formula (I).

112. The method of any one of claims 1-89, wherein the pharmaceutical composition comprises a mixture of an S,S diastereomer of a compound of Formula (I) and an R,R diastereomer of a compound of Formula (I).

113. The method of any one of claims 1-89, wherein the pharmaceutical composition comprises a mixture of brensocatib, or a pharmaceutically acceptable salt thereof and (2S)—N-{(1R)-1-Cyano-2-[4-(3-methyl-2-oxo-2,3-dihydro-1,3-benzoxazol-5-yl)phenyl]ethyl}-1,4-oxazepane-2-carboxamide: 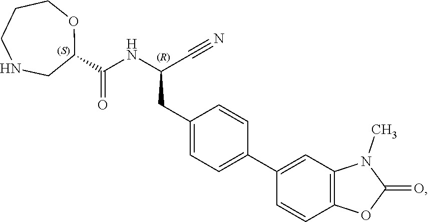
or a pharmaceutically acceptable salt thereof.

114. The method of any one of claims 1-89, wherein the pharmaceutical composition comprises a mixture of brensocatib, or a pharmaceutically acceptable salt thereof and (2R)—N-{(1S)-1-Cyano-2-[4-(3-methyl-2-oxo-2,3-dihydro-1,3-benzoxazol-5-yl)phenyl]ethyl}-1,4-oxazepane-2-carboxamide: 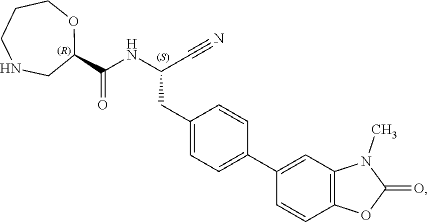
or a pharmaceutically acceptable salt thereof.

115. The method of any one of claims 1-89, wherein the pharmaceutical composition comprises a mixture of brensocatib, or a pharmaceutically acceptable salt thereof and (2R)—N—{(1R)-1-Cyano-2-[4-(3-methyl-2-oxo-2,3-dihydro-1,3-benzoxazol-5-yl)phenyl]ethyl}-1,4-oxazepane-2-carboxamide: 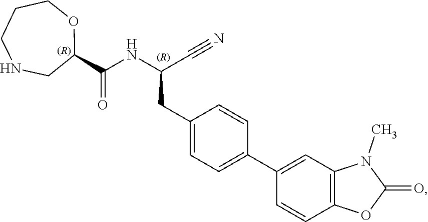
or a pharmaceutically acceptable salt thereof.

116. The method of any one of claims 1-115, wherein the pharmaceutical composition is administered once-a-day, twice-a-day, or every other day during the administration period.

117. The method of any one of claims 1-116, wherein the pharmaceutical composition is administered once-a-day during the administration period.

118. The method of any one of claims 1-117, wherein the pharmaceutical composition is administered orally during the administration period.

119. The method of any one of claims 1-117, wherein the pharmaceutical composition is administered parenterally, enterally, or through a nasogastric tube during the administration period.

120. The method of any one of claims 1-119, wherein the compound of Formula (I) is present in the pharmaceutical composition from about 1 mg to about 100 mg.

121. The method of claim 120, wherein the compound of Formula (I) is present in the pharmaceutical composition at about 10 mg.

122. The method of claim 120, wherein the compound of Formula (I) is present in the pharmaceutical composition at about 25 mg.

123. The method of claim 120, wherein the compound of Formula (I) is present in the pharmaceutical composition at about 40 mg.

124. The method of claim 120, wherein the compound of Formula (I) is present in the pharmaceutical composition at about 5 mg to about 50 mg.

125. The method of claim 120, wherein the compound of Formula (I) is present in the pharmaceutical composition at about 10 mg to about 40 mg.

126. The method of claim 120, wherein the compound of Formula (I) is present in the pharmaceutical composition at about 10 mg to about 25 mg.

127. The method of any one of claims 1-126, wherein the administration period is about 30 days, about 1 month, about 2 months, about 3 months, about 4 months, about 5 months, about 6 months, about 7 months, about 8 months, about 9 months, about 10 months, about 11 months, about 12 months, about 18 months, about 24 months or about 30 months.

128. The method of any one of claims 1-126, wherein the administration period is at least about 30 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 18 months, at least about 24 months or at least about 30 months.

129. The method of any one of claims 1-126, wherein the administration period is about 30 days, about 1 month, about 2 months, about 3 months, about 4 months, about 5 months, about 6 months, about 7 months, about 8 months, about 9 months, about 10 months, about 11 months, about 12 months, about 18 months, about 24 months, about 30 months, about 36 months, about 4 years, about 5 years, about 10 years, about 15 years or about 20 years.

130. The method of any one of claims 1-126, wherein the administration period is at least about 30 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 18 months, at least about 24 months, at least about 30 months, at least about 36 months, at least about 4 years, at least about 5 years, at least about 10 years, at least about 15 years or at least about 20 years.

131. The method of any one of claims 1-126, wherein the administration period is about 6 months.

132. The method of any one of claims 1-126, wherein the administration period is about 12 months.

133. The method of any one of claims 1-126, wherein the administration period is from about 6 months to about 36 months.

134. The method of any one of claims 1-126, wherein the administration period is from about 12 months to about 36 months.

135. The method of any one of claims 1-126, wherein the administration period is from about 18 months to about 36 months.

136. The method of any one of claims 1-126, wherein the administration period is from about 1 year to about 50 years.

137. The method of any one of claims 1-126, wherein the administration period is from about 1 year to about 40 years.

138. The method of any one of claims 1-126, wherein the administration period is from about 1 year to about 30 years.

139. The method of any one of claims 1-126, wherein the administration period is from about 1 year to about 25 years.

140. The method of any one of claims 1-126, wherein the administration period is from about 1 year to about 20 years.

141. The method of any one of claims 1-126, wherein the administration period is from about 1 year to about 15 years.

142. The method of any one of claims 1-126, wherein the administration period is from about 1 year to about 10 years.

143. The method of any one of claims 1-126, wherein the administration period is from about 1 year to about 5 years.

144. The method of any one of claims 1-126, wherein the administration period is from about 1 year to about 3 years.

145. The method of any one of claims 1-126, wherein the administration period is from about 1 year to about 2 years.

146. The method of any one of claims 1-126, wherein the administration period is from about 2 years to about 15 years.

147. The method of any one of claims 1-126, wherein the administration period is from about 2 years to about 10 years.

148. The method of any one of claims 1-126, wherein the administration period is from about 2 years to about 8 years.

149. The method of any one of claims 1-126, wherein the administration period is from about 2 years to about 5 years.

150. The method of any one of claims 1-126, wherein the administration period is from about 2 years to about 4 years.

151. The method of any one of claims 1-150, wherein the subject is at a risk of developing HS.

152. The method of claim 151, wherein the subject at a risk of developing HS is or was repeatedly exposed to tobacco smoke; has a family history of HS; or a combination thereof.

153. The method of any one of claims 1-152, wherein the HS is associated with the presence or development of one or more of: acne, arthritis, diabetes, metabolic syndrome, inflammatory bowel disease, obesity, or any combination thereof.

154. The method of any one of claims 11-153, wherein prior to the administration period is immediately prior to the administration period.

155. The method of any one of claims 11-153, wherein prior to the administration period is from about 1 day to about 14 days prior to the administration period.

156. The method of any one of claims 11-153, wherein prior to the administration period is from about 1 day to about 10 days prior to the administration period.

157. The method of any one of claims 11-153, wherein prior to the administration period is from about 1 day to about 7 days prior to the administration period.

158. The method of any one of claims 11-153, wherein prior to the administration period is from about 1 day to about 4 days prior to the administration period.
